(19) United States
(12) Patent Application Publication
(10) Pub. No.: US 2025/0008961 A1
(43) Pub. Date: Jan. 9, 2025

(54) PAENIBACILLUS STRAINS PRODUCING LOW AMOUNTS OF DAB COMPRISING COMPOUNDS

(71) Applicant: BASF SE, Ludwigshafen (DE)

(72) Inventors: Tobias May, Ludwigshafen (DE); Judith Zimmermann, Limburgerhof (DE); Bogdan Tokovenko, Ludwigshafen (DE); Daniel Christoph Heinrich, Ludwigshafen (DE); Reinhard Stierl, Limburgerhof (DE); Andrea Herold, Ludwigshafen (DE)

(21) Appl. No.: 18/711,970

(22) PCT Filed: Nov. 11, 2022
(86) PCT No.: PCT/EP2022/081565

§ 371 Date: May 21, 2024

(30) Foreign Application Priority Data
Nov. 22, 2021 (EP) 21209627.5

(51) Int. Cl. A01N 63/25; A01P 3/00; C12N 1/20; C12N 9/10

(52) U.S. Cl. CPC A01N 63/25; A01P 3/00; C12N 1/20; C12N 9/1096; C12Y 206/01076

(57) ABSTRACT

The present invention relates to Paenibacillus species strains producing low amounts of com-pounds requiring L-2,4-diaminobutanoate as precursor and exopolysaccharides comprising low amounts of compounds requiring L-2,4-diaminobutanoate as precursor, as well as mixtures, compositions and methods comprising these Paenibacillus species strains and/or exopolysac-charides.

### FIELD

The present invention relates to Paenibacillus species strains producing low amounts of compounds requiring L-2,4-diaminobutanoate as precursor and exopolysaccharides comprising low amounts of compounds requiring L-2,4-diaminobutanoate as precursor, as well as mixtures, compositions and methods comprising these Paenibacillus species strains and/or exopolysaccharides.

### BACKGROUND

The Gram-positive bacteria of the genus Paenibacillus are known for a multitude of potential uses. Paenibacillus species strains have been described as production hosts for recombinant proteins, basic chemicals or useful polymers, like exopolysaccharides (Heinze et al, 2020, Draft Genome Sequence of Paenibacillus polymyxa DSM 292, a Gram-positive, Spore-Forming Soil Bacterium with High Biotechnological Potential. Microbiology resource announcements, 9(11), e00071), (Schilling et al, 2020, Engineering of the 2,3-butanediol pathway of Paenibacillus polymyxa DSM 365, Metabolic Engineering, Volume 61, Pages 381-388 and Ruetering et al, 2017, Tailor-made exopolysaccharides-CRISPR-Cas9 mediated genome editing in Paenibacillus polymyxa, Synthetic Biology, 2(1): ysx007).

In particular Paenibacillus strains isolated from plants and their rhizosphere have further shown to have additional uses in agriculture. These comprise the use as biological pesticide to protect plants from plant pathogenic fungi and bacteria, promotion of plant growth via the production of phytohormones and mobilization of nutrients, biological nitrogen fixation and induction of systemic acquired resistance. Many of these uses are summarized in Jeong et al, 2019, Chronicle of a Soil Bacterium: Paenibacillus polymyxa E681 as a Tiny Guardian of Plant and Human Health, Frontiers in Microbiology, Volume 10, Article 467, using Paenibacillus polymyxa E681 as an example.

It is clear that many of these applications will not need the full spectrum of abilities that Paenibacillus strains can provide. For example, the ability to produce antifungal or antibacterial substances can be seen as production of unnecessary or even unwanted side products when Paenibacillus strains are used for the production of recombinant enzymes, exopolysaccharides or basic chemicals. For these applications it can also be considered as an advantage, if the Paenibacillus species strain used comprises a small genome to lessen the metabolic burden created by the production of unnecessary proteins or the production of compounds which are unnecessary for the production of the protein or compound of interest and would be considered as unwanted side products which in some cases may even cause additional measures and cost in down-stream-processing to either purify the compound of interest or to dispose the side products in a save way. A lower production of unnecessary proteins and unwanted side products may also free up metabolic resources to enhance the production capacity for the respective protein or compound of interest. Microbial production strains of other species have been modified to produce less unwanted side products, see for example WO9822598, WO9960137 and US2017121719. Most of these efforts have been concentrated on other microorganisms than Paenibacillus. Thus, there remains a need for improved Paenibacillus strains which produce lower amounts of unnecessary proteins and/or side products.

Applications of Paenibacillus strains in agriculture also suffer sometimes from specific features of Paenibacillus strains. For example, there is an interest to use mixtures of microorganisms in agriculture, either to support plant growth, by providing a diverse and stable rhizosphere microbial community of microbial strains with plant growth enhancing features as for example in WO201845004, or to enhance particular functions, like protection against fungal pathogens by combining microbial strains having complementing features. Mixtures of two or several Bacillus species or mixtures of Bacillus species with Pseudomonas species and mixtures of several Pseudomonas species have been described for example in WO2016109424, CN109699682 and WO2017178529. A further motivation is to provide protection against fungal pathogens and an enhanced supply of nitrogen to the plants. Mixtures between a Bacillus strain with antifungal effect and Bradyrhizobium strains for enhanced nitrogen supply is described in WO2020263812.

Paenibacillus strains have been described to produce several antibacterial substances, which suppress the growth on gram-negative and/or gram-positive bacteria, like polymyxins, tridecaptins, polypeptin, paenilipoheptins, octapeptins, olipeptin, gavaserine, saltavalin, several lantibiotics, like paenicidin, paenibacillin, paenilan, and gatavalin. Most Paenibacillus strains have the capability to produce several, but not all of them. This means that most Paenibacillus strains can negatively impact the growth of other useful gram-negative or gram-positive bacteria which may either form natural protective bacterial communities on the plant surface, like the ones described in Helfrich et al., 2018, Bipartite interactions, antibiotic production and biosynthetic potential of the Arabidopsis leaf microbiome, Nature Microbiology, VOL 3, pages 909-919, or may intentionally been added to the Paenibacillus strains to enhance positive effects on plant growth and plant health. Examples for such mixtures are mixtures in which the antifungal and nitrogen fixating ability of Paenibacillus strains, as described in Ali et al, 2021, Functional Analysis and Genome Mining Reveal High Potential of Biocontrol and Plant Growth Promotion in Nodule-Inhabiting Bacteria Within Paenibacillus polymyxa Complex Front. Microbiol., 18, Article 618601, is combined with the antifungal and nitrogen fixing ability of Rhizobia, as described in Das et al, 2017, Rhizobia: a potential biocontrol agent for soilborne fungal pathogens, Folia Microbiol, 62, pages 425-435, are combined.

Accordingly, there is a need to develop improved Paenibacillus strains which produce less compounds which interfere with the growth of attractive mixing partners selected from Gram-negative or Gram-positive bacteria. This causes also a need to develop methods to create and identify such strains without laborious recombinant methods or intensive chemical mutagenesis. The methods and strains provided herein address these needs.

### SUMMARY OF THE INVENTION

The invention comprises Paenibacillus species strains comprising only one genetic locus comprising at least 80% sequence identity to a polynucleotide sequence of any one of SEQ ID NOs: 222, 223, 224, 225, 413, 414, 415, 416, 417, 418 and not comprising an open reading frame for a diaminobutyrate-2-oxoglutarate-transaminase comprising an amino acid sequence of at least 80% sequence identity to SEQ ID NO: 217. Preferably the Paenibacillus species strains do not comprise a polynucleotide sequence encoding an amino acid sequence comprising at least 80% sequence identity to SEQ ID NO: 1.

In one embodiment the Paenibacillus species strains have an ANI-Value of at least 99.9% to the Paenibacillus polymyxa strain LU17007 deposited as DSM 26970 or to the Paenibacillus polymyxa strain SB3-1. In a further embodiment the Paenibacillus species strains are derived from a parent strain comprising an open reading frame encoding an amino acid sequence of at least 80% sequence identity to SEQ ID NO: 217 and an open reading frame encoding an amino acid sequence of at least 80% sequence identity to SEQ ID NO: 409, preferably encoded by a polynucleotide sequence of at least 80% sequence identity to SEQ ID NO: 411, and an open reading frame encoding an amino acid sequence of at least 80% sequence identity to SEQ ID NO: 410 preferably encoded by a polynucleotide sequence of at least 80% sequence identity to SEQ ID NO: 412.

The invention comprises also mixtures comprising a) a pure culture of a Paenibacillus species strain which does not produce polymyxin and b) a pure culture of a microorganism which is sensitive to polymyxin. Preferably the Paenibacillus species strain of a) does not produce a polymyxin or a tridecaptin, or does not produce a polymyxin and does not produce a tridecaptin, and the second microorganism of b) is sensitive to a polymyxin or a tridecaptin or is sensitive to a polymyxin and a tridecaptin. Preferably the microorganism of b) is not sensitive to the presence of the Paenibacillus species strain of a) but is sensitive to the presence of the parent strain of the Paenibacillus species strain of a). In one embodiment the mixture comprises a) a pure culture of a Paenibacillus species strain which does not comprise an open reading frame for a diaminobutyrate-2-oxoglutarate-transaminase comprising an amino acid sequence of at least 90% sequence identity to at least one of SEQ ID NO: 217, 218, 219, 220 or 221 and b) a pure culture of a microorganism which is sensitive to at least one of polymyxin, tridecaptin, polypeptin, paenilipoheptin, octapeptin, jolipeptin, gavaserin or saltavalin, preferably is sensitive to at least one of polymyxin, tridecaptin or both.

Preferably the Paenibacillus species strain of a) comprises no or only one locus encoding an amino acid sequence comprising at least 90% sequence identity to SEQ ID NO: 1 and does not produce a polymyxin or a tridecaptin or does not produce a polymyxin or a tridecaptin and does preferably not produce a DAB comprising compound.

In a further embodiment the mixture comprises a microorganism of b) selected from Azotobacter, Azospirillum, Pseudomonas, Paraburkholderia, Rhizobium, Bradyrhizobium, Bacillus or Streptomyces, preferably selected from the species Pseudomonas koreensis, Pseudomonas gessardii, Pseudomonas chlororaphis, Paraburkholderia phytofirmans, Rhizobium phaseoli, Rhizobium radiobacter, Bradyrhizobium japonicum, Bacillus licheniformis, Bacillus subtilis, Bacillus amyloliquefaciens, Bacillus licheniformis, Bacillus mycoides, Bacillus firmus, Bacillus thuringiensis, Bacillus pumilus, Bacillus simplex, Streptomyces griseoviridis and Streptomyces lydicus.

The invention comprises also Paenibacillus species strains producing more of at least one glutamine comprising fusaricidin, paeniprolixin or paeniserine than of its respective asparagine comprising counterpart, preferably the Paenibacillus species strains produce more fusaricidin LiF04b (Fus B) than fusaricidin LiF04a (Fus A).

The invention comprises also agrochemical compositions comprising at least one of the Paenibacillus species strains of the invention.

The invention comprises also whole culture broths, cell-free culture broths, spores or cell-free extracts of a Paenibacillus species strain of the invention.

Further embodiments of the invention are methods for controlling, suppressing or preventing fungal infection of plants comprising the steps of a) providing a pure culture of a Paenibacillus species strain of the invention or a mixture comprising such a Paenibacillus species strain or a whole culture broth, a cell-free culture broth or a cell-free extract of a Paenibacillus species strain of the invention or an agrochemical composition comprising at least one of the above and b) applying an effective amount of the pure culture of a Paenibacillus species strain of the invention, or a mixture comprising such a Paenibacillus species strain or a whole culture broth, a cell-free culture broth or a cell-free extract of a Paenibacillus species strain of the invention or an agrochemical composition comprising at least one of the above to the plant pathogenic fungi, their habitat, the host plants, plant propagation material or the soil used to grow the plant.

The invention also provides isolated Paenibacillus exopolysaccharides which does not comprise colistin, tridecaptin M or both and preferably do not comprise a polymyxin or tridecaptin and even more preferred do not comprise any DAB comprising compound.

In one embodiment the Paenibacillus exopolysaccharides are comprised in a composition also comprising at least one of a) to c) being: a) an auxiliary, b) a pure culture of microorganism which is sensitive to the presence of colistin, tridecaptin M or both, preferably is sensitive to the presence of at least one polymyxin or at least one tridecaptin and even more preferred is sensitive to the presence of at least one DAB comprising compound, or c) a fertilizer. The invention comprises also plant propagation material comprising a pure culture of a Paenibacillus species strain of the invention, or an agrochemical composition or a mixture comprising such a strain, or a whole culture broth, a cell-free culture broth or a cell-free extract comprising or prepared from such strain, or isolated Paenibacillus exopolysaccharides of the invention.

The invention further provides for a method of production of a valuable product comprising, a) culturing a Paenibacillus species strain of the invention and b) harvesting the valuable product. Preferably the valuable product is a) an enzyme or a protein, b) an antifungal compound, c) an exopolysaccharide, d) one or more DAB comprising compounds or e) 2,3-Butanediol, lactic acid or acetoin.

Another embodiment of the invention is a method to create a Paenibacillus species strain of the invention, comprising the following steps of a) providing a Paenibacillus species parent strain, b) providing stress to a culture of the Paenibacillus species parent strain of a), c) culturing the stressed Paenibacillus species parent strain of b) and d) isolating a pure culture of a Paenibacillus species strain not producing colistin, tridecaptin M or both and preferably not comprising a polymyxin or a tridecaptin and even more preferred not comprising any DAB comprising compound.

### BRIEF DESCRIPTION OF THE DRAWINGS

FIG. 1 shows an agar plate in which colonies of mutated LU17007 are overlaid with a top agar culture of E. coli after 48 h of incubation. A mutant colony of LU17007 lacking an inhibition zone against E. coli is marked with a square.

### DETAILED DESCRIPTION

The present invention may be understood more readily by reference to the following detailed description of the embodiments of the invention and the examples included herein. Although the present invention will be described with respect to particular embodiments, this description is not to be construed in a limiting sense.

### Definitions

Unless otherwise noted, the terms used herein are to be understood according to conventional usage by those of ordinary skill in the relevant art.

Before describing in detail exemplary embodiments of the present invention, definitions important for understanding the present invention are given. Unless stated otherwise or apparent from the nature of the definition, the definitions apply to all compounds, methods and uses described herein.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

Throughout this application, various publications are referenced. The disclosures of all of these publications and those references cited within those publications in their entireties are hereby incorporated by reference into this application in order to more fully describe the state of the art to which this invention pertains.

It is to be understood that the term “comprising” is not limiting. For the purposes of the present invention the term “consisting of” is considered to be a preferred embodiment of the term “comprising”. If hereinafter a group is defined to comprise at least a certain number of embodiments, this is meant to also encompass a group which preferably consists of these embodiments only. “Parent” sequence (also called “parent enzyme” or “parent protein”) is the starting sequences for introduction of changes (e.g. by introducing one or more amino acid substitutions) of the sequence resulting in “variants” of the parent sequences. Thus, the term “enzyme variant” or “sequence variant” or “protein variant” are used in reference to parent enzymes that are the origin for the respective variant enzymes. Therefore, parent enzymes include wild type enzymes and variants of wild-type enzymes which are used for development of further variants. Variant enzymes differ from parent enzymes in their amino acid sequence to a certain extent.

In describing the variants of the present invention, the abbreviations for single amino acids used according to the accepted IUPAC single letter or three letter amino acid abbreviation is used.

“Amino acid alteration” as used herein refers to amino acid substitution, deletion, or insertion.

“Substitutions” are described by providing the original amino acid followed by the number of the position within the amino acid sequence, followed by the substituted amino acid. For example, the substitution of histidine at position 120 with alanine is designated as “His120Ala” or

“H120A”. Substitutions can also be described by merely naming the resulting amino acid in the variant without specifying the amino acid of the parent at this position, e.g., “X120A” or “120A” or “Xaa120Ala” or “120Ala”.

“Deletions” are described by providing the original amino acid followed by the number of the position within the amino acid sequence, followed by *. Accordingly, the deletion of glycine at position 150 is designated as “Gly150*” or G150*”. Alternatively, deletions are indicated by e.g. “deletion of D183 and G184”.

“Insertions” are described by providing the original amino acid followed by the number of the position within the amino acid sequence, followed by the original amino acid and the additional amino acid. For example, an insertion at position 180 of lysine next to glycine is designated as “Gly180GlyLys” or “G180GK”. When more than one amino acid residue is inserted, such as e.g. a Lys and Ala after Gly180 this may be indicated as: Gly180GlyLysAla or G195GKA.

In cases where a substitution and an insertion occur at the same position, this may be indicated as S99SD+S99A or in short S99AD. In cases where an amino acid residue identical to the existing amino acid residue is inserted, it is clear that degeneracy in the nomenclature arises. If for example a glycine is inserted after the glycine in the above example this would be indicated by G180GG. Variants comprising multiple alterations are separated by “+”, e.g., “Arg170Tyr+Gly195Glu” or “R170Y+G195E” representing a substitution of arginine and glycine at positions 170 and 195 with tyrosine and glutamic acid, respectively. Alternatively, multiple alterations may be separated by space or a comma, e.g., R170Y G195E or R170Y, G195E respectively. Where different alternative alterations can be introduced at a position, the different alterations are separated by a comma, e.g., “Arg170Tyr, Glu” and R170T, E, respectively, represents a substitution of arginine at position 170 with tyrosine or glutamic acid. Alternative substitutions at a particular position can also be indicated as X120A,G,H, 120A,G,H, X120A/G/H, or 120A/G/H. Alternatively, different alterations or optional substitutions may be indicated in brackets, e.g., Arg170[Tyr, Gly] or Arg170{Tyr, Gly} or in short R170 [Y, G] or R170 {Y, G}.

A “synthetic” or “artificial” compound is produced by in vitro chemical and/or enzymatic synthesis.

The term “native” (or naturally occurring or wildtype or endogenous) cell or organism or polynucleotide or polypeptide refers to the cell or organism or polynucleotide or polypeptide as found in nature (i.e., without there being any human intervention). For the purposes of the invention, “recombinant” (or transgenic) with regard to a cell or an organism means that the cell or organism contains a heterologous polynucleotide which is introduced by man by gene technology and with regard to a polynucleotide includes all those constructions brought about by man by gene technology/recombinant DNA techniques in which either
(a) the sequence of the polynucleotide or a part thereof, or
(b) one or more genetic control sequences which are operably linked with the polynucleotide, including but not limited thereto a promoter, or
(c) both a) and b) are not located in their wildtype genetic environment or have been modified by man.

The term “heterologous” (or exogenous or foreign or recombinant or non-native) polypeptide is defined herein as a polypeptide that is not native to the host cell, a polypeptide native to the host cell in which structural modifications, e.g., deletions, substitutions, and/or insertions, have been made by recombinant DNA techniques to alter the native polypeptide, or a polypeptide native to the host cell whose expression is quantitatively altered or whose expression is directed from a genomic location different from the native host cell as a result of manipulation of the DNA of the host cell by recombinant DNA techniques, e.g., a stronger promoter. Similarly, the term “heterologous” (or exogenous or foreign or recombinant or non-native) polynucleotide refers to a polynucleotide that is not native to the host cell, a polynucleotide native to the host cell in which structural modifications, e.g., deletions, substitutions, and/or insertions, have been made by recombinant DNA techniques to alter the native polynucleotide, or a polynucleotide native to the host cell whose expression is quantitatively altered as a result of manipulation of the regulatory elements of the polynucleotide by recombinant DNA techniques, e.g., a stronger promoter, or a polynucleotide native to the host cell, but integrated not within its natural genetic environment as a result of genetic manipulation by recombinant DNA techniques. With respect to two or more polynucleotide sequences or two or more amino acid sequences, the term “heterologous” is used to characterize that the two or more polynucleotide sequences or two or more amino acid sequences are naturally not occurring in the specific combination with each other.

Variant polynucleotide and variant polypeptide sequences may be defined by their sequence identity when compared to a parent sequence. Sequence identity usually is provided as “% sequence identity” or “% identity”. For calculation of sequence identities, in a first step a sequence alignment is produced. According to this invention, a pairwise global alignment is produced, meaning that two sequences are aligned over their complete length, which is usually produced by using a mathematical approach, called alignment algorithm.

According to the invention, the alignment is generated by using the algorithm of Needleman and Wunsch (J. Mol. Biol. (1979) 48, p. 443-453). Preferably, the program “NEEDLE” (The European Molecular Biology Open Software Suite (EMBOSS)) is used for the purposes of the current invention, with using the programs default parameter (polynucleotides: gap open=10.0, gap extend=0.5 and matrix=EDNAFULL; polypeptides: gap open=10.0, gap extend=0.5 and matrix=EBLOSUM62). After aligning two sequences, in a second step, an identity value is determined from the alignment produced. For this purpose, the %-identity is calculated by dividing the number of identical residues by the length of the alignment region which is showing the respective sequence of the present invention over its complete length multiplied with 100: %-identity=(identical residues/length of the alignment region which is showing the respective sequence of the present invention over its complete length)*100.

For calculating the percent identity of two nucleic acid sequences the same applies as for the calculation of percent identity of two amino acid sequences with some specifications. For nucleic acid sequences encoding for a protein the pairwise alignment shall be made over the complete length of the coding region of the sequence of this invention from start to stop codon excluding introns. Introns present in the other sequence, to which the sequence of this invention is compared, may also be removed for the pairwise alignment. Percent identity is then calculated by %-identity=(identical residues/length of the alignment region which is showing the sequence of the invention from start to stop codon excluding introns over their complete length)*100. After aligning two sequences, in a second step, an identity value is determined from the alignment produced.

Moreover, the preferred alignment program for nucleic acid sequences implementing the Needleman and Wunsch algorithm (J. Mol. Biol. (1979) 48, p. 443-453) is “NEEDLE” (The European Molecular Biology Open Software Suite (EMBOSS)) with the programs default parameters (gapopen=10.0, gapextend=0.5 and matrix=EDNAFULL).

Sequences, having identical or similar regions with a sequence of this invention, and which shall be compared with a sequence of this invention to determine % identity, can easily be identified by various ways that are within the skill in the art, for instance, using publicly available computer methods and programs such as BLAST, BLAST-2, available for example at NCBI.

Variant polypeptides may be defined by their sequence similarity when compared to a parent sequence. Sequence similarity usually is provided as “% sequence similarity” or “%-similarity”. % sequence similarity takes into account that defined sets of amino acids share similar properties, e.g. by their size, by their hydrophobicity, by their charge, or by other characteristics. Herein, the exchange of one amino acid with a similar amino acid may be called “conservative mutation”. Similar amino acids according to the invention are defined as follows, which shall also apply for determination of %-similarity according to this invention, which is also in accordance with the BLOSUM62 matrix as for example used by program “NEEDLE”, which is one of the most used amino acids similarity matrix for database searching and sequence alignments:
Amino acid A is similar to amino acids S
Amino acid D is similar to amino acids E; N
Amino acid E is similar to amino acids D; K; Q
Amino acid F is similar to amino acids W; Y
Amino acid H is similar to amino acids N; Y
Amino acid I is similar to amino acids L; M; V
Amino acid K is similar to amino acids E; Q; R
Amino acid L is similar to amino acids I; M; V
Amino acid M is similar to amino acids I; L; V
Amino acid N is similar to amino acids D; H; S
Amino acid Q is similar to amino acids E; K; R
Amino acid R is similar to amino acids K; Q
Amino acid S is similar to amino acids A; N; T
Amino acid T is similar to amino acids S
Amino acid V is similar to amino acids I; L; M
Amino acid W is similar to amino acids F; Y
Amino acid Y is similar to amino acids F; H; W

Conservative amino acid substitutions may occur over the full length of the sequence of a polypeptide sequence of a functional protein such as an enzyme. In one embodiment, such mutations are not pertaining the functional domains of an enzyme. In one embodiment, conservative mutations are not pertaining the catalytic centers of an enzyme. For calculation of sequence similarity, in a first step a sequence alignment is produced as described above. After aligning two sequences, in a second step, a similarity value is determined from the alignment produced. For this purpose, the %-similarity is calculated by dividing the number of identical residues plus the number of similar residues by the length of the alignment region which is showing the sequence of the invention over its complete length multiplied with 100: %−similarity=[(identical residues+similar residues)/length of the alignment region which is showing the sequence of the invention over its complete length]*100.

The term “ANI-Value” is based on the Average Nucleotide Identity (ANI) between two microbial genomes. An ANI-Value is provided in %, in which higher values point to a high similarity between the genomes in question.

The ANI-Value as used herein refers to a Value calculated based on the FastANI method described for the version FastANI (v1.0) in Jain, C. et al. High throughput ANI analysis of 90K prokaryotic genomes reveals clear species boundaries. Nat Commun 9, 5114 (2018). https://doi.org/i0.1038/s41467-018-07641-9. The ANI-Value is preferably calculated based on the most recent version of the method. In case different versions provide different ANI-Values, the value of the version FastANI v1.33 will be decisive. Usually, a value of less than 95% is used to distinguish the genomes of two different species. However, this value is flexible and depends on the individual situation. For example, based on the results of Murray, C. S., Gao, Y. & Wu, M. Re-evaluating the evidence for a universal genetic boundary among microbial species. Nat Commun 12, 4059 (2021). https://doi.org/10.1038/s41467-021-24128-2, an ANI-Value of less than 95% person will not be correct in every case to determine that two microorganisms in question belong to different species.

For calculating the ANI-Value, the following parameters are set to I is set to 3 Kbp, T is set to 50, wherein the two sequence sets to be compared need to have at least 150Kbp homologous genome sequence. The ANI-Value calculated with the FastANI method is usually robust even if the analyzed sequence data sets have variable assembly quality, completeness, and contamination. Preferably the datasets to be compared have at least 15 times, preferably 20 times, coverage and are at least 99% complete with have less than 2% contamination. The sequence datasets can be created using Illumina HiSeq 2500 DNA sequencing technology or similar technologies available in the art. Preferably sequencing technologies creating long-read sequences are used, like Nanopore Sequencing or Single Molecule, Real-Time (SMRT) Sequencing, also known under the name PACBIO® sequencing. Even more preferred, the datasets are prepared using a combination of Illumina HiSeq 2500 DNA sequencing and a technology creating long-read sequences.

The terms “coverage”, “complete, and “contamination” are terms used in the art to describe the quality of a certain data set for a sequenced genome. A 15 times coverage refers to how many times, on average, a certain position in a genome has been sequenced e.g., for a genome size of 5 megabases (MB), 100 MB of DNA sequencing from the given genome is required to have 15 times sequencing coverage on average at each position along the genome. The completeness of a sequenced genome and the amount of contamination is calculated by using defined sets of marker genes. Preferably by using the CheckM or BUSCO methods, see Parks et al 2015, CheckM: assessing the quality of microbial genomes recovered from isolates, single cells, and metagenomes. Genome research, 25(7), 1043-1055, and Felipe et al, 2015, BUSCO: assessing genome assembly and annotation completeness with single-copy orthologs, Bioinformatics, Volume 31, Issue 19, 1 Oct. 2015, Pages 3210-3212. Preferably these methods are used in their most recent version of the method. In case different versions of the methods provide different results, the results of the version CheckM v1.1. and BUSCO v5.2.2 will be decisive. In case the CheckM and BUSCO methods provide different results, the result of the BUSCO method will be decisive.

The term “genetic locus” is a specific, position on a microbial chromosome or plasmid where a particular gene or genetic marker is located. A duplication of genes results in the occurrence of the same genetic sequence, or a very similar one, in two different genetic loci in the genome or plasmid.

The term “open reading frame” or “ORF” refers to the part of a gene sequence, which encodes a protein or polypeptide sequence. It is a continuous stretch of codons that may begin with a start codon (usually AUG) and ends at a stop codon (usually UAA, UAG or UGA). An ATG codon within the ORF (not necessarily the first) may indicate where translation starts. The transcription termination site is located after the ORF, beyond the translation stop codon.

The term “spores” refers to at least one dormant (at application) but viable reproductive unit of a bacterial species. It is further recognized that the spores disclosed herein are produced via culturing of bacteria and are usually harvested from the fermentation broth by techniques used in the art, like centrifugation or filtration. They can therefore comprise a combination of vegetative cells and forespores (cells in an intermediate stage of spore formation); a combination of forespores and spores; or a combination of forespores, vegetative cells and/or spores, as well as other solid components of the fermentation broth.

The term “pure culture” refers to a community of microorganisms which belong to the same strain and show only phenotypic and/or genotypic variation which is due to different physiological states, epigenetic modifications, or the natural frequency of mutation of the specific strain. In many cases will a pure culture of a microorganism show some degree of domestication, meaning that it comprises genotypic traits, which lead to advantageous phenotypic traits for cultivation on or in artificial growth media or for industrial production and appear rarely under growth conditions of the natural habitat. A pure culture of a given microorganism may be combined with one or more pure cultures of other microorganisms. However, based on the genetic similarity of all cells of a single pure culture will mixtures of pure cultures of many different strains, e.g. up to 100 different pure cultures, not reach the genetic complexity of a natural microbiome or a sample of microorganisms freshly isolated from nature. The Paenibacillus species strains herein as well as their potential mixing partners will preferably be used as pure cultures.

The term “strain” refers a group of microorganisms exhibiting the same phenotypic and/or genotypic traits, except for variations based on the natural rate of mutation, which share the same lineage back to a single isolate, distinct from those of other isolates or strains of the same species and have, except for small variations, the same genome sequence.

The term “whole culture broth” refers to a combination to the result of remaining components of a culture broth used to grow a microorganism, the produced biomass of the microorganism, in particular cells and/or spores of the microorganism, and other components produced during culturing of the microorganism, like secondary compounds produced by the microorganism. Usually, the whole culture broth is concentrated and purified to a certain degree, e.g. in case the whole culture broth is concentrated via filtration. Accordingly, the term whole culture broth also encompasses these modified forms as long as they are liquid to a certain degree and comprise biomass of the microorganism and components of the culture broth used to grow the microorganism. The biomass of the microorganism usually comprises a mixture of living, dormant and dead cells. In some cases, the biomass of the microorganism is killed by intention.

The term “cell-free culture broth” refers to the liquid parts of a culture broth used to grow a microorganism after the biomass of the microorganism has been removed by techniques known in the art. The cell-free culture broth may be further concentrated after removal of the biomass and may further be purified but will still comprise components of the medium used to grow the microorganism and compounds produced by the microorganism. Thus, the term cell-free culture broth” is intended to encompass such modified forms of the liquid part of the culture broth. Preferably, the cell-free culture broth will still comprise some parts of the microorganism, like remaining DNA, proteins or characteristic combination of secondary compounds, which allow to identify which species and/or strain had been used to produce the cell-free culture broth.

The term “cell-free extract” refers to an extract of the vegetative cells, spores and/or the whole culture broth of a microorganism comprising cellular metabolites produced by the respective microorganism obtainable by cell disruption methods known in the art such as solvent based (e.g. organic solvents such as alcohols sometimes in combination with suitable salts), temperature-based, application of shear forces, cell disruption with an ultrasonicator. The desired extract may be concentrated by conventional concentration techniques such as drying, evaporation, centrifugation or alike. Certain washing steps using organic solvents and/or water-based media may also be applied to the crude extract preferably prior to use.

The term “DAB comprising compound” or DAB comprising compounds” refer to compounds in which L-2,4-diaminobutanoate (DAB) is used as a precursor or intermediate during their biosynthesis in a way that at least some atoms of the DAB molecules are incorporated in a compound. DAB comprising compounds in a stricter sense are compounds selected from polymyxins, tridecaptins, polypeptin, paenilipoheptins, octapeptins, jolipeptin, gavaserin and saltavalin.

The term “polymyxins” refers to lipo-decapeptides containing from five to six DAB residues, wherein seven amino acid residues form a cyclic component, while the other three extend from one of the cyclic residues as a linear chain terminating in either 6-methyloctanoic acid or 6-methylheptanoic acid at the N-terminus. Polymyxins are well known in the art and comprise several variants based on differences in amino acid or fatty acid composition. Known polymyxin variants are polymyxin A, B, B-I, C, D, E1 (colistin A), E2 (colistin B), F, M (mattacin), P, S1, and T1. The frequently used variant name polymyxin B refers to a mixture composed of polymyxins B1, B1-1, B2, B3, and B6. Structural information on these variants can for example be found in Stephen A. Cochrane and John C. Vederas, 2016, Lipopeptides from Bacillus and Paenibacillus spp.: A Gold Mine of Antibiotic Candidates Medicinal Research Reviews, 36, No. 1, 4-31 and in Niu et al. 2013, Polymyxin P is the active principle in suppressing phytopathogenic Erwinia spp. by the biocontrol rhizobacterium Paenibacillus polymyxa M-1. BMC Microbiol; 13:137.

Gene clusters responsible for polymyxin biosynthesis (pmx cluster) have been described for several Paenibacillus strains. The pmx cluster encompasses five genes, of which pmxA, pmxB, and pmxE encode the polymyxin synthetase, whereas pmxD and pmxC are involved in polymyxin transport. Further information on the pmx cluster can be found in Aleti et al (2015) Genome mining: prediction of lipopeptides and polyketides from Bacillus and related Firmicutes, Comput Struct Biotechnol J 13:192-203, and in Choi et al, 2009, Identification of a polymyxin synthetase gene cluster of Paenibacillus polymyxa and heterologous expression of the gene in Bacillus subtilis. J Bacteriol 191(10):3350-3358.

The term “tridecaptins” refers to linear tridecapeptides with a combination of L and D amino acids that are acylated with a 6-methyloctanoic or a C9 to C13 β-hydroxy fatty acid side chain linked to their N-terminal amino acid. Tridecaptins are well known and at least ten variants have been described in the art. These variants are tridecaptin Aα, Aβ, A4, Bα, Bβ, Bγ, Bδ, Cα, Cβ, E and M. Structural information on tridecaptin variants can be found in Stephen A. Cochrane and John C. Vederas, 2016, Lipopeptides from Bacillus and Paenibacillus spp.: A Gold Mine of Antibiotic Candidates Medicinal Research Reviews, 36, No. 1, 4-31. Gene clusters responsible for tridecaptin biosynthesis (tri cluster) have been described for several Paenibacillus strains. Tri cluster generally encompass five genes which have been named trbA, trbB, trbC, trbD and trbE or triA to triE or trmA to trmE by different authors. Sequence information tri cluster have been, for example, deposited under GenBank accession no.: KF111342 and KR422398.

TrbA encodes a putative thioesterase, TrbB and TrbC encode ABC transporter proteins and TrbD and TrbE encode contain ten and three adenylation domains for the synthesis of the polypeptide chain. Sequence searches can identify variants of these clusters, e.g. the variant for the production of tridecaptin M, see Jangra et al., 2019, Tridecaptin M, a new variant discovered in mud bacterium, shows activity against colistin- and extremely drug-resistant Enterobacteriaceae, Antimicrob Agents Chemother 63:e00338-19, which also describes the structure of tridecaptin M. The structure of tridecaptin E is described in Vater et al., 2018, Genome Mining of the Lipopeptide Biosynthesis of Paenibacillus polymyxa E681 in Combination with Mass Spectrometry: Discovery of the Lipoheptapeptide Paenilipoheptin, ChemBioChem, 19, 744-753.

The term “polypeptin” refers to natural compounds which have a general structure composed of a cyclic nonapeptide moiety and a R-hydroxy fatty acid. They have been described by several research groups and are also known under the names pelgipeptins, permetins or pelgipeptins, see Mountford et al., 2017, The first total synthesis and solution structure of a polypeptin, PE2, a cyclic lipopeptide with broad spectrum antibiotic activity Org. Biomol. Chem., 15, 7173. Additional information on the structures of polypeptins and an exemplary gene cluster (plp cluster) can be found in Qian et al., 2012, Identification and functional analysis of gene cluster involvement in biosynthesis of the cyclic lipopeptide antibiotic pelgipeptin produced by Paenibacillus elgii, BMC Microbiology, 12:197, and in Stephen A. Cochrane and John C. Vederas, 2016, Lipopeptides from Bacillus and Paenibacillus spp.: A Gold Mine of Antibiotic Candidates Medicinal Research Reviews, 36, No. 1, 4-31.

The term “paenilipoheptins” refers to natural compounds comprising a cyclic heptapeptide moiety rich in aromatic amino-acid components (Phe, Tyr, Trp) and contain a C12 or C13 β-amino fatty acid that is linked through its amino group with the COOH group of the C-terminal Glu. One paenilipoheptin has the sequence of Ser-DAB-Trp-Val-Phe-Tyr-Glu in its heptapeptide structure. Further structural information on paenilipoheptins can be found in Vater et al., 2018, Genome Mining of the Lipopeptide Biosynthesis of Paenibacillus polymyxa E681 in Combination with Mass Spectrometry: Discovery of the Lipoheptapeptide Paenilipoheptin, ChemBioChem, 19, 744-753

The term “octapeptins” refers to natural DAB comprising compounds comprising eight monomers having a general structure composed of a cyclic heptapeptide moiety and a side chain. The group of octapeptins comprise several well-known variants: octapeptin A1 (synonym EM 49β), A2 and A3 unified under the synonym EM49α, octapeptin B1 (synonymEM49δ), B2 and B3 unified under the synonym EM 496, octapeptin B5 (synonym battacin), octapeptin Cl (synonym 333-25), D, Bu-1880, Y-8495, and Bu-2470, see Meyers et al., 1976, A nomenclature proposal for the octapeptin antibiotics. J Antibiot (Tokyo) 29(11):1241-1242, Sugawara K, et al. 1983. Bu-2470, a new peptide antibiotic complex. II. Structure determination of Bu-2470 A, B1, B2a and B2b. J. Antibiot. 36: 634-638 and Qian et al, 2012, battacin (octapeptin B5), a New Cyclic Lipopeptide Antibiotic from Paenibacillus tianmuensis Active against Multidrug-Resistant Gram-Negative Bacteria Antimicrobial Agents and Chemotherapy, Vol. 56/3, 1458-1465. An octapeptin biosynthetic cluster has been described in B. circulans ATCC 31805 in Velkov et al., 2018, Structure, function, and biosynthetic origin of octapeptin antibiotics active against extensively drug-resistant gramnegative bacteria. Cell Chem Biol 25(4):380-391 e5 to comprise three nonribosomal peptide synthetases OctA, OctB, and OctC

The term “gavaserin” refers to a cyclic octapeptid of a molecular masse of 911 Daltons, comprising Glu, Ala, 2×Val, Ser, 3×DAB and octanoic acid. More information can be found in Pichard et al., 1995, Gavaserin and saltavalin, new peptide antibiotics produced by Bacillus polymyxa. FEMS Microbiol Lett 133(3):215-218. No structural data about this LP as well as gene encoding its production are available.

The term “saltavalin” refers to a non-cyclic peptide having a molecular masse of about 903 Daltons and comprising Ser, Ala, 2×Leu, 2×Thr, Val, and 2×DAB acid with no fatty acid component attached. More information can be found in Pichard et al., 1995, Gavaserin and saltavalin, new peptide antibiotics produced by Bacillus polymyxa. FEMS Microbiol Lett 133(3):215-218. No structural data about this LP as well as gene encoding its production are available.

The term “jolipeptin” refers to cyclic polypeptides comprising Ser, Ala, Val, Gly, Glu and DAB. Further features and antimicrobial spectrum of Jolipeptin has been described in Ito and Koyama, 1971, LOCALIZATION OF JOLIPEPTIN AND COLISTIN IN THEIR PRODUCING STRAIN, BACILLUS POLYMYXA VAR. COLISTINUS, THE JOURNAL OF ANTIBIOTICS, VOL. XXV NO. 2, 147 to 148 and Ito and Koyama, 1972, JOLIPEPTIN, A NEW PEPTIDE ANTIBIOTIC I. ISOLATION, PHYSICO-CHEMICAL AND BIOLOGICAL CHARACTERISTICS, THE JOURNAL OF ANTIBIOTICS, VOL. XXV NO. 5, 304 to 308.

The term “fusaricidin compounds” refers to a group of about 80 cyclic or acyclic lipohexapeptides containing a 15-Guanidino-3-hydroxypentadecanoic acid (GHPD), 17-Guanidino-3-hydroxyheptadecanoic acid (GHHD) or a 19-Guanidino-3-hydroxynonadecanoic acid (GHND) component, see Vater et al., 2017, Fusaricidins from Paenibacillus polymyxa M-1, a family of lipohexapeptides of unusual complexity—a mass spectrometric study, J. Mass Spectrom., 52, 7-15. The hexapeptide part comprises, in single amino acid code, a T or S in position 1, a I or V in position 2, a V or I or Y or F in position 3, a T or S in position 4, a N or Q at position 5 and an A on position 6. Fusaricidin compounds comprising 15-Guanidino-3-hydroxypentadecanoic acid component are called fusaricidins. Fusaricidin compounds comprising a 17-Guanidino-3-hydroxyheptadecanoic acid or a 19-Guanidino-3-hydroxynonadecanoic acid are usually called paeniprolixins. Fusaricidin compounds comprising a S in the hexapeptide part are usually called paeniserines. Some examples for fusaricidins, paeniprolixins and paeniserines are listed in Table 1.

TABLE 1

| Name | Form | Fatty Acid | Amino acid sequence |
|---|---|---|---|
| Fusaricidin LiF03a | cyclic | GHPD | TVYTNA |
| Fusaricidin LiF03b | cyclic | GHPD | TVYTQA |
| Fusaricidin LiF04a (FUS A) | cyclic | GHPD | TVVTNA |
| Fusaricidin LiF04b (FUS B) | cyclic | GHPD | TVVTQA |
| Fusaricidin LiF05a | cyclic | GHPD | TVITNA |
| Fusaricidin LiF05b | cyclic | GHPD | TVITQA |
| Fusaricidin LiF06a | cyclic | GHPD | TIVTNA |
| Fusaricidin LiF06b | cyclic | GHPD | TIVTQA |
| Fusaricidin LiF07a | cyclic | GHPD | TVFTNA |
| Fusaricidin LiF07b | cyclic | GHPD | TVFTQA |
| Paeniprolixin C1 | cyclic | GHHD | TIITNA |
| Paeniprolixin C2 | cyclic | GHHD | TIITQA |
| Paeniserine A1 | cyclic | GHPD | SVVTNA |
| Paeniserine A2 | cyclic | GHPD | SVVTQA |

Further examples are described in Qiu et al., 2019, Identification of fusaricidins from the antifungal microbial strain Paenibacillus sp. MS2379 using ultra-high performance liquid chromatography coupled to quadrupole time-of-flight mass spectrometry, Journal of Chromatography A, 1586, 91-100.

Several gene cluster (fus cluster) for the production of fusaricidin compounds have been described in the art, see for example: Li et Jensen, 2008, Nonribosomal Biosynthesis of Fusaricidins by Paenibacillus polymyxa PKB1 Involves Direct Activation of a D-Amino Acid, Chemistry & Biology 15, 118-127 and Choi et al., 2008, Identification and functional analysis of the fusaricidin biosynthetic gene of Paenibacillus polymyxa E681. Biochem. Biophys. Res. Commun. 365, 89-95. doi: 10.1016/j.bbrc.2007.10.147. Targeted modifications of fusaricidin compound producing genes have also been done, see for example: Han et al., 2012, Site-directed modification of the adenylation domain of the fusaricidin nonribosomal peptide synthetase for enhanced production of fusaricidin analogs. Biotechnol Lett.; 34:1327-34.

The term “levan” refers to a linear β-(2→6)-linked polyfructan. Levans are produced from sucrose-based substrate by a transfructosylation reaction of levansucrase (beta-2,6-fructan:D-glucose-fructosyl transferase, EC 2.4.1.10). Further information on levan can be found in Liu et al. Preparation, antioxidant and antitumor activities in vitro of different derivatives of levan from endophytic bacterium Paenibacillus polymyxa EJS-3. Food Chem. Toxicol. 2012, 50, 767-772.

The term “paenan” refers to a heteropolysaccharide comprising glucose, mannose, galactose, and glucuronic acid in the ratio 3.5:2:1:0.1 produced by Paenibacillus species. Composition and rheological properties of paenan have been described in Rutering et al, 2018, Rheological characterization of the exopolysaccharide Paenan in surfactant systems, Carbohydrate Polymers, 181, 719-726 and in Rutering et al., 2016, Controlled production of polysaccharides-exploiting nutrient supply for levan and heteropolysaccharide formation in Paenibacillus sp., Carbohydrate Polymers, 148, 326-334.

The term “curdlan” refers to a polysaccharide composed exclusively of beta-1,3-linked glucose residues. More information on Curdlan production in Paenibacillus species can be found in Rafigh et al, 2014, Optimization of culture medium and modeling of curdlan production from Paenibacillus polymyxa by RSM and ANN, International Journal of Biological Macromolecules 70, 463-473

### DETAILED DESCRIPTION

Described herein are Paenibacillus species strains comprising only one genetic locus comprising in the order of rising preference, at least 95%, 96%, 97%, 98%, 99%, or 100% sequence identity to a polynucleotide sequence of any one of SEQ IN NOs: 222, 223, 224, 225, 413, 414, 415, 416, 417, 418 and not comprising an open reading frame for a diaminobutyrate-2-oxoglutarate-transaminase comprising, in the order of rising preference, an amino acid sequence of at least 90%, 92,%, 93%, 94%, 95%, 96%, 97%, 98%, 99% or 100% sequence identity to SEQ ID NO: 217.

In one embodiment the Paenibacillus species strains comprise only one genetic locus comprising in the order of rising preference, at least 95%, 96%, 97%, 98%, 99%, or 100% sequence identity to a polynucleotide sequence of any one of SEQ ID NOs: 224, 225, 413, 414, 415, 416, 417, 418 and does not comprise an open reading frame for a diaminobutyrate-2-oxoglutarate-transaminase comprising, in the order of rising preference, an amino acid sequence of at least 90%, 92,%, 93%, 94%, 95%, 96%, 97%, 98%, 99% or 100% sequence identity to SEQ ID NO: 217.

In one embodiment the Paenibacillus species strains comprise only one genetic locus comprising in the order of rising preference, at least 98%, or 99%, or 100% sequence identity to a polynucleotide sequence of any one of SEQ IN NOs: 413, 414, 415, 416, 417, 418 and does not comprise an open reading frame for a diaminobutyrate-2-oxoglutarate-transaminase comprising, in the order of rising preference, an amino acid sequence of at least 90%, 92,%, 93%, 94%, 95%, 96%, 97%, 98%, 99% or 100% sequence identity to SEQ ID NO: 217.

The strains may belong to any Paenibacillus species but belong preferably to a species selected from Paenibacillus polymyxa, Paenibacillus peoriae, Paenibacillus sp. aloe-11, Paenibacillus kribbensis and Paenibacillus terrae. More preferred they are Paenibacillus polymyxa strains.

It is not uncommon in the art that certain microorganisms are reclassified to belong to a different species, or that certain strains of unknown phylogenetic relationship are reclassified to belong to a certain species, for example see Kwak et al., 2020, Genome-based reclassification of Paenibacillus jamilae Aguilera et al. 2001 as a later heterotypic synonym of Paenibacillus polymyxa (Prazmowski 1880) Ash et al. 1994, Int. J. Syst. Evol. Microbiol.; 70:3134-3138, or Velazquez et al. 2020, Paenibacillus ottowii sp. nov. isolated from a fermentation system processing bovine manure, Int. J. Syst. Evol. Microbiol.; 70:1463-1469. The same is true for the genus Paenibacillus, which has formerly been classified as belonging to the genus Bacillus, see Ash et al. 1993. Molecular identification of rRNA group 3 bacilli (Ash, Farrow, Wallbanks and Collins) using a PCR probe test. Proposal for the creation of a new genus Paenibacillus. Antonie Van Leeuwenhoek. 64(3-4): 253-260.

These reclassifications of microorganisms are often based on comparisons of sequenced genomes of the microorganisms in question. Accordingly, the term Paenibacillus species strain includes also strains, which have a different genus name, but should belong to the Paenibacillus genus based on similarity of its genome to other Paenibacillus species strains. The degree of similarity between two genomes is frequently expressed in ANI-Values. More than 200 Paenibacillus genomes, including 73 Paenibacillus polymyxa genomes, have been sequenced and deposited in international databases, for example GenBank. Examples are: P. polymyxa ATCC 842, also known under the strain number DSM 36, GenBank AccNo: CP049783.1,
P. polymyxa Sb3-1 GenBank AccNo: CP010268.1,
P. polymyxa M-152 GenBank AccNo: CP034141.1,
P. polymyxa M1 GenBank AccNo: HE577054.1,
P. polymyxa SC2 GenBank AccNo: CP002213.2,
P. polymyxa SQR-21 GenBank AccNo: CP006872.1,
P. polymyxa HY96-2 GenBank AccNo: CP025957.1,
P. polymyxa YC0136 GenBank AccNo: CP017967.3,
P. polymyxa E681 GenBank AccNo: CP048794.1,
P. polymyxa CR1 GenBank AccNo: CP006941.2,
P. polymyxa J GenBank AccNo: CP015423.1,
P. polymyxa ZF197 GenBank AccNo: CP042272.1,
P. polymyxa WLY78 NCBI Reference Sequence: NZ_ALJV01000125.1,
P. ottowii strain MS2379 NCBI Reference Sequence: NZ_VIJZ00000000.1,
P. peoriae ZF390 GenBank AccNo: CP061172.1,
P. peoriae HS311 GenBank AccNo: CP011512.1,
P. kribbensis AM49 GenBank AccNo: CP020028.1,
P. kribbensis PS04 GenBank AccNo: CP041731.1,
P. terrae HPL-003 GenBank AccNo: CP003107.1,
P. sp. Aloe-11 NCBI Reference Sequence: NZ_JH601056.1,

Further Paenibacillus genomes can readily be accessed by sequencing deposited strains, like P. polymyxa LU17007 deposited as DSM 26970, which has an ANI-Value of about 98% to the genomes of P. polymyxa Sb3-1, P. polymyxa M-1, or P. polymyxa SC2 and an ANI-Value of 99% to P. polymyxa M-152.

For the purpose herein, a given Paenibacillus species strain is considered to belong to the species Paenibacillus polymyxa if it comprises an ANI-Value of at least 88% to the genome of the type strain ATCC 842. Based on this definition, Paenibacillus species strain Paenibacillus ottowii strain MS2379 is also considered to be a Paenibacillus polymyxa strain. A comparison of Paenibacillus polymyxa strain genomes via ANI-Values is provided in: Wang et al. 2020, Comparative genome analysis and mining of secondary metabolites of Paenibacillus polymyxa, Genes Genet. Syst. 95, p. 141-15G0.

A given Paenibacillus species strain is considered to belong to the species Paenibacillus peoriae, if it comprises an ANI-Value of at least 88% to the genome of P. peoriae ZF390.

A given Paenibacillus species strain is considered to belong to the species Paenibacillus kribbensis if it comprises an ANI-Value of at least 88% to the genome of P. kribbensis AM49.

A given Paenibacillus species strain is considered to belong to the species Paenibacillus terrae if it comprises an ANI-Value of at least 88% to the genome of P. terrae HPL-003.

A given Paenibacillus species strain is considered to belong to the species Paenibacillus sp. Aloe-11, if it comprises an ANI-Value of at least 88% to the genome of P. sp. Aloe-11

In case a genome has an ANI-Value of at least 88% two the genomes of two of these strains, it is considered to belong to the species to which is has the higher ANI-Value.

Preferred Paenibacillus strains have an ANI-Value of, in the order of rising preference, at least 95%, 96%, 97%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100.0% to the genome of P. polymyxa Sb3-1, P. polymyxa M-1, or P. polymyxa SC2 and preferably to P. polymyxa SB3-1,

In another embodiment, the Paenibacillus strains have an ANI-Value of, in the order of rising preference, at least 95%, 96%, 97%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100.0%. to the genome of P. polymyxa LU17007 deposited as DSM 26970.

Diaminobutyrate-2-oxoglutarate-transaminases are well known in the art. These enzymes catalyse the reaction of: L-glutamate and L-aspartate-4-semialdehyde to 2-oxoglutarate and L-2,4-diaminobutanoate. Organisms which do not comprise a functional version of these enzymes are not able to produce L-2,4-diaminobutanoate or only to a low amount. Examples for such organisms are organisms either having no coding region for an enzyme capable to perform the synthesis reaction or comprise coding regions for an enzyme with a much lower activity or stability or express such enzyme in a low amount. Preferably the organism do not comprise a coding region for such enzyme.

Examples for amino acid sequences of diaminobutyrate-2-oxoglutarate-transaminases are provided herein with the SEQ ID NO: 217, 218, 219, 220 and 221. A skilled person will readily be able to identify the sequence encoding a diaminobutyrate-2-oxoglutarate-transaminase of a given strain of interest. Table 2 shows % sequence identity values between the amino acid sequences of SEQ ID NO: 217, 218, 219, 220 and 221.

TABLE 2

| | 217_LU17007 | 218_E681 | 219_HPL-003 | 220_M1 | 221_Sb3-1 |
|---|---|---|---|---|---|
| 217_LU17007 | 100.0% | 94.5% | 93.1% | 99.3% | 99.5% |
| 218_E681 | — | 100.0% | 93.3% | 94.3% | 94.5% |
| 219 HPL-003 | — | — | 100.0% | 92.9% | 93.1% |
| 220_M1 | — | — | — | 100.0% | 99.8% |
| 221_Sb3-1 | — | — | — | — | 100.0% |

Paenibacillus strains can use the L-2,4-diaminobutanoate (DAB) produced by the diaminobutyrate-2-oxoglutarate-transaminase as building block to produce several natural compounds which show inhibitory effects to Gram-negative and/or Gram-positive bacteria. Natural compounds comprising DAB as essential building block are for example polymyxin, tridecaptin, polypeptin, paenilipoheptin, octapeptin, jolipeptin, gavaserin and saltavalin. Paenibacillus strains producing one or more of these DAB comprising compounds are usually resistant to the DAB compounds produced by them.

Paenibacillus strains which are unable to produce DAB are unable to produce these compounds, if not supplied with DAB via the growth medium.

It was observed that Paenibacillus species strains of the invention can be created by selecting a recombination event between two genetic loci in a parent strain, which comprises two loci, which are both predicted to encode non-ribosomal-peptide-synthetases (NPRS) with sequence similarity to NPRS TrdD (synonymous TriD or TrmD) of the tridecaptin gene cluster, e.g. the one disclosed in Jangra et al, 2019, Tridecaptin M, a New Variant Discovered in Mud Bacterium, Shows Activity against Colistin- and Extremely Drug-Resistant Enterobacteriaceae, Antimicrobial Agents and Chemotherapy, Volume 63, Issue 6, e00338-19.

The genomic fragment of the parent strain between these two loci comprises a locus encoding a diaminobutyrate-2-oxoglutarate-transaminase, so that a deletion event caused by a recombination between the two NPRS encoding sequences results in deletion of the locus for the diaminobutyrate-2-oxoglutarate-transaminase and the reduction of the two NPRS encoding loci to only one newly created locus with sequence similarity to a tridecaptin producing NPRS. Such recombination events may occur spontaneously e.g. by laboratory evolutions and can then be selected for, but can also be triggered by techniques known in the art, for example by mutagenesis and selection for the expected phenotype or can be constructed using targeted genetic engineering methods. Preferably, the two loci undergoing recombination show sequence similarity to the polynucleotide sequences of SEQ ID NO: 411 and 412, preferably encoding parts of the NPRS sequences of SEQ ID NOs: 409 and 410, respectively. Preferably the recombination results in a polynucleotide sequence with similarity to any one of SEQ ID Nos: 222, 223, 224, 225, 413, 414, 415, 416, 417 and 418, and preferably resulting in a frame shift or deletions in the encoded amino acid sequence in comparison to the amino acid sequence of SEQ ID NO: 1.

Preferably, the parent strain comprises an open reading frame encoding an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 217 and an open reading frame encoding an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 409, preferably encoded by a polynucleotide sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 411, and an open reading frame encoding an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 410 preferably encoded by a polynucleotide sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 412. Preferably the recombination happens between the polynucleotide comprising a polynucleotide sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 411 and a polynucleotide comprising a polynucleotide sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 411.

Preferable, the locus comprising a polynucleotide sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 412 and the locus comprising a polynucleotide sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 411 share, in the order of rising preference, at least 90%, 91%, 92%, 93%, 94%, 95%, 96%, 97%, 98%, 99%, or 100% sequence identity to each other.

Preferably, the two polynucleotides undergoing recombination encode an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 1.

Alternatively, the Paenibacillus strains of the invention can be created by targeted manipulation of Paenibacillus strains via recombinant techniques, e.g. using gene editing techniques like CRISPR, thereby avoiding the deletion of the whole genomic fragment between the two loci capable to undergo recombination.

Independent of its way of creation, i.e. via recombinant techniques or spontaneous or triggered recombination, the invention comprises Paenibacillus polymyxa strains not comprising a polynucleotide sequence encoding an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 1 and not comprising an open reading frame for a diaminobutyrate-2-oxoglutarate-transaminase comprising an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 217 from a parent strain comprising an open reading frame for a diaminobutyrate-2-oxoglutarate_transaminase comprising an amino acid sequence of, in the order of rising preference, at least 80%, 81%, 82%, 83%, 84%, 85%, 86%, 87%, 88%, 89%, 90%, 91%, 92%, 93%, 94%, 91%, 92%, 93%, 94%, 95%, 96%, 97%, 98%, 99%, or 100% sequence identity to SEQ ID NO: 217 and at least two loci each one comprising, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to at least one of the nucleotide sequences of SEQ ID NO: 222, 223, 224, 225, 413, 414, 415, 416, 417 and 418, and wherein the two loci comprise, in the order of rising preference, at least 90%, 91%, 92%, 93%, 94%, 95%, 96%, 97%, 98%, 99%, or 100% sequence identity to each other

The parent strain has preferably an ANI-Value of, in the order of rising preference, at least 90%, 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of at least one strain selected from the group comprising: P. polymyxa LU17007, P. polymyxa SQR-21, P. polymyxa M1, P. polymyxa SC2, P. polymyxa HY96-2, P. polymyxa YC0136, P. polymyxa E681, P. polymyxa CR1, P. polymyxa J, P. polymyxa ZF197, P. polymyxa WLY78, P. ottowii strain MS2379, P. peoriae ZF390, P. peoriae HS311, P. kribbensis AM49, P. kribbensis PS04, P. terrae HPL-003, P. sp. aloe-11

The parent strain has preferably an ANI-Value of, in the order of rising preference, at least 90%, 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. polymyxa LU17007 deposited as DSM 26970.

The parent strain has preferably an ANI-Value of, in the order of rising preference, at least 90% 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. polymyxa SQR-21.

The parent strain has preferably an ANI-Value of, in the order of rising preference, at least 90% 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. polymyxa M-1.

The parent strain has preferably an ANI-Value of, in the order of rising preference, at least 90% 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. polymyxa SC2.

The parent strain has preferably an ANI-Value of, in the order of rising preference, at least 90% 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. polymyxa HY96-2.

The parent strain has preferably an ANI-Value of, in the order of rising preference, at least 90% 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. polymyxa YC0136.

The parent strain has preferably an ANI-Value of, in the order of rising preference, at least 90% 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. polymyxa E681.

The parent strain has preferably an ANI-Value of, in the order of rising preference, at least 90% 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. polymyxa CR1.

The parent strain has preferably an ANI-Value of, in the order of rising preference, at least 90% 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. polymyxa J.

The parent strain has preferably an ANI-Value of, in the order of rising preference, at least 90% 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. polymyxa ZF197.

The parent strain has preferably an ANI-Value of, in the order of rising preference, at least 90% 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. polymyxa WLY78.

The parent strain has preferably an ANI-Value of, in the order of rising preference, at least 90% 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. ottowii strain MS2379.

The parent strain has preferably an ANI-Value of, in the order of rising preference, at least 90% 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. peoriae ZF390.

The parent strain has preferably an ANI-Value of, in the order of rising preference, at least 90% 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. peoriae HS311.

The parent strain has preferably an ANI-Value of, in the order of rising preference, at least 90% 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. kribbensis AM49.

The parent strain has preferably an ANI-Value of, in the order of rising preference, at least 90% 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. kribbensis PS04.

The parent strain has preferably an ANI-Value of, in the order of rising preference, at least 90% 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. terrae HPL-003.

The parent strain has preferably an ANI-Value of, in the order of rising preference, at least 90% 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. sp. Aloe-11.

The parent strain has preferably an ANI-Value of, in the order of rising preference, at least 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. polymyxa LU17007 deposited as DSM 26970.

The parent strain has preferably an ANI-Value of, in the order of rising preference, at least 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. polymyxa SQR-21.

In case the Paenibacillus strains of the invention are created via recombination, the deleted genome fragment of the parent strain can be of considerable size.

In the case that the parent strain is Paenibacillus polymyxa strain LU17007 deposited as DSM 26970 or Paenibacillus polymyxa strain SB3-1 or similar strains having an ANI-Value of, in the order of rising preference, at least 90%, 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of Paenibacillus polymyxa strain LU17007 deposited as DSM 26970 or Paenibacillus polymyxa strain SB3-1, the genomic fragment lost due to the recombination event comprises a plurality, preferably at least 147, of coding regions, which encode polypeptides which have, in the order of rising preference, at least 90%, 91%, 92%, 93%, 94%, 95%, 96%, 97%, 98%, 99%, or 100% sequence identity
a) to the amino acid sequences of SEQ ID NOs: 2, 3, 5, 7, 8, 9, 10, 11, 12, 13, 14, 15, 16, 17, 18, 19, 20, 21, 22, 23, 25, 27, 28, 30, 31, 32, 34, 36, 37, 38, 39, 40, 41, 44, 45, 47, 48, 50, 51, 52, 53, 54, 55, 58, 60, 61, 62, 63, 64, 65, 66, 67, 68, 69, 70, 72, 77, 78, 79, 80, 81, 82, 83, 84, 86, 87, 89, 91, 92, 93, 94, 95, 96, 97, 98, 100, 101, 102, 103, 104, 105, 107, 108, 109, 110, 111, 112, 113, 114, 115, 116, 117, 118, 119, 120, 122, 124, 125, 126, 127, 128, 129, 130, 132, 133, 134, 135, 136, 137, 138, 139, 140, 141, 142, 143, 144, 145, 146, 147, 148, 149, 150, 151, 152, 153, 154, 155, 156, 157, 158, 159, 161, 162, 163, 164, 165, 166, 167, 169, 170, 174, 175, 176, 177, 178, 180, 181, or
b) to the amino acid sequences of SEQ ID NOs: 241, 242, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 309, 310, 311, 312, 313, 314, 315, 316, 317, 318, 319, 320, 321, 322, 323, 324, 325, 326, 327, 328, 329, 330, 331, 332, 333, 347, 348, 349, 350, 351, 352, 353, 354, 355, 356, 357, 358, 360, 363, 363, 364, 365, 366, 367, 369, 370, 371, 372, 373, 374, 375, 376, 377, 378, 379, 380, 381, 382, 383, 384, 385, 387, 388, 389, 390, 391, 392, 393, 394, 395, 396, 397, 398, 399, 400, 401, 402, 403, 404, 405, 406, 407.

Preferably the genomic fragment lost due to the recombination event comprises at least one coding sequence of, in the order of rising preference, at least 90%, 91%, 92%, 93%, 94%, 95%, 96%, 97%, 98%, 99%, or 100% sequence identity for each one of the amino acid sequences listed above under a) or b), respectively.

TABLE 3
Polypeptides encoded on the genomic fragment of Paenibacillus polymyxa strain LU17007 deposited as DSM 26970 and Paenibacillus polymyxa
strain SB3-1, respectively. The table lists the predicted function of the encoded polypeptides, the SEQ ID NOs comprising the respective
amino acid sequences and the percent sequence identities between similar polypeptides encoded in the genome fragments.

| | predicted | | predicted | % seq. |
|---|---|---|---|---|
| SEQ ID | protein function | SEQ ID | protein function | ident. |
| 2 | bacitracin_synthetase_3 | 407 | non-ribosomal_peptide_synthetase | 99.6 |
| 3 | protein_flp | 406 | serine_hydrolase | 99.1 |
| 5 | endonuclease_yhcR | 405 | endonuclease | 98.5 |
| 7 | hypothetical_protein_22823_23171 | 404 | hypothetical_protein | 100.0 |
| 8 | transcription_elongation_factor_greA | 403 | transcription_elongation_factor_GreA | 100.0 |
| 9 | putative_protein_yrxA | 402 | transcription_repressor_NadR | 100.0 |
| 10 | aminotransferase_class_V | 401 | IscS_subfamily_cysteine_desulfurase | 99.2 |
| 11 | quinolinate_synthetase_A | 400 | quinolinate_synthase_NadA | 98.6 |
| 12 | hypothetical_protein | 399 | hypothetical_protein | 99.1 |
| 13 | inner_membrane_protein_oxaA | 398 | membrane_protein_insertase_YidC | 99.6 |
| 14 | GCN5-related_N-acetyltransferase | 397 | GCN5_family_acetyltransferase | 99.6 |
| 15 | lipid_phosphate_phosphohydrolase | 396 | phosphatase_PAP2_family_protein | 99.6 |
| 16 | putative_protein_ywoA | 395 | undecaprenyl-diphosphatase | 97.2 |
| 17 | putative_transcriptional_regulatory_protein_ykoG | 394 | response_regulator_transcription_factor | 99.2 |
| 18 | two-component_sensor_histidine_kinase | 393 | HAMP_domain-containing_histidine_kinase | 99.3 |
| 19 | putative_protein_ydhO_Flags_Precursor | 392 | C40_family_peptidase | 99.4 |
| 20 | glycosyl_transferase_group | 391 | glycosyltransferase_family_4_protein | 99.7 |
| 21 | Succinylglutamate_{—} | 390 | succinylglutamate_desuccinylase_{—} | 95.4 |
| | desuccinylase | | aspartoacylase_family | |
| 22 | mannose-1-phosphate_{—} | 389 | mannose-1-phosphate_{—} | 98.7 |
| | guanylyltransferase | | guanylyltransferase | |
| 23 | hypothetical_protein | 388 | hypothetical_protein | 100.0 |
| 25 | endospore_coat-associated_protein_yheC | 387 | YheC_YheD_family_protein | 96.4 |
| 27 | ATP-binding_protein | 385 | ABC_transporter_ATP-binding_protein | 99.6 |
| 28 | putative_transporter | 384 | AEC_family_transporter | 99.0 |
| 30 | hypothetical_protein | 383 | rhodanese-related_sulfurtransferase | 99.0 |
| 31 | glycosyl_hydrolase_family_27 | 382 | glycoside_hydrolase_family_27_protein | 99.3 |
| 32 | PfkB_family_carbohydrate_kinase | 38- | carbohydrate_kinase_family_protein | 98.6 |
| 34 | topology_modulation_protein | 380 | adenylate_kinase | 100.0 |
| 36 | prolyl-tRNA_synthetase | 379 | proline--tRNA_ligase | 99.3 |
| 37 | spermidine_synthase_1 | 378 | fused_MFS_spermidine_synthase | 100.0 |
| 38 | hypothetical_protein | 377 | YebC_PmpR_family_DNA- | 100.0 |
| | | | binding_transcriptional_regulator | |
| 39 | HTH-type_transcriptional_{—} | 376 | TetR_AcrR_family_{—} | 100.0 |
| | repressor_dhaR | | transcriptional_regulator | |
| 40 | phenazine_biosynthesis_{—} | 375 | nuclear_transport_{—} | 100.0 |
| | protein_phzB | | factor_2_family_protein | |
| 41 | prestalk_A_{—} | 374 | NmrA_family_{—} | 99.7 |
| | differentiation_protein_A | | NAD-binding_protein | |
| 44 | putative_protein | 372 | Cys-tRNA_deacylase | 98.7 |
| 45 | 50S_ribosomal_protein_L7_L12 | 371 | ribosomal_protein_L7_L12 | 96.4 |
| 47 | xylose_repressor | 370 | ROK_family_protein | 100.0 |
| 48 | 6-phospho-beta-glucosidase | 369 | glycoside_hydrolase_family_1_protein | 99.8 |
| 50 | phosphodiesterase_yfcE | 367 | phosphodiesterase | 99.5 |
| 51 | Agmatine_deiminase | 366 | agmatine_deiminase_family_protein | 99.1 |
| 52 | putative_carbon-nitrogen_hydrolase | 365 | N-carbamoylputrescine_amidase | 99.7 |
| 53 | hypothetical_protein | 364 | hypothetical_protein | 94.3 |
| 54 | NADH_flavin_oxidoreductase | 363 | FAD-dependent_oxidoreductase | 100.0 |
| 55 | NADH_flavin_oxidoreductase | 363 | FAD-dependent_oxidoreductase | 99.2 |
| 58 | hypothetical_protein | 360 | immunity_63_family_protein | 98.0 |
| 60 | ferrichrome_transport_system_permease_protein_fhuB | 358 | iron_ABC_transporter_permease | 98.8 |
| 61 | Vitamin_B12_import_system_permease_protein_btuC | 357 | iron_ABC_transporter_permease | 99.7 |
| 62 | NAD | 356 | NADH_oxidoreductase | 97.9 |
| 63 | putative_HTH-type_{—} | 355 | helix-turn-helix_{—} | 100.0 |
| | transcriptional_regulator_ytcD | | transcriptional_regulator | |
| 64 | protein_PhnB | 354 | VOC_family_protein | 100.0 |
| 65 | hypothetical_protein | 353 | DUF3934_family_protein | 98.4 |
| 66 | pseudouridylate_synthase | 352 | pseudouridine_synthase | 99.6 |
| 67 | spore_germination_protein_A2 | 351 | endospore_germination_permease | 100.0 |
| 68 | hypothetical_protein | 350 | hypothetical_protein | 100.0 |
| 69 | spore_germination_protein_KC_Flags_Precursor | 349 | GerC_family_spore_germination_protein | 99.8 |
| 70 | spore_germination_protein_KA | 348 | spore_germination_protein | 99.6 |
| 72 | hydrolase_alpha_beta_fold_family | 347 | alpha_beta_hydrolase | 98.9 |
| 77 | hydroxyacylglutathione_hydrolase | 333 | MBL_fold_metallo-hydrolase | 90.5 |
| 78 | hypothetical_protein | 332 | hypothetical_protein | 97.1 |
| 79 | ATP-dependent_Clp_protease_{—} | 331 | ATP-dependent_Clp_endopeptidase_{—} | 99.5 |
| | proteolytic_subunit | | proteolytic_subunit_ClpP | |
| 80 | RNA_polymerase_sigma_factor | 330 | RNA_polymerase_sigma_factor | 98.8 |
| 81 | alpha_beta_hydrolase | 329 | alpha_beta_fold_hydrolase | 99.2 |
| 82 | hypothetical_protein | 328 | DUF4303_domain-containing_protein | 97.7 |
| 83 | methionine_aminopeptidase | 327 | type_I_methionyl_aminopeptidase | 99.6 |
| 84 | hypothetical_protein | 326 | TetR_family_transcriptional_regulator | 93.5 |
| 86 | putative_protein_yraL | 325 | hypothetical_protein | 87.5 |
| 87 | lipase 87271_88288 | 324 | alpha_beta_hydrolase | 98.8 |
| 89 | hypothetical_protein | 323 | thioredoxin_domain-containing_protein | 99.5 |
| 91 | methyl-accepting_chemotaxis_protein_mcpA_H1 | 322 | methyl-accepting_chemotaxis_protein | 99.2 |
| 92 | cheY-like_receiver | 321 | hybrid_sensor_histidine_{—} | 97.9 |
| | | | kinase_response_regulator | |
| 93 | sensory_transduction_protein_regX3 | 320 | response_regulator_transcription_factor | 100.0 |
| 94 | diaminobutyrate-2- | 319 | diaminobutyrate--2- | 99.5 |
| | oxoglutarate_transaminase | | oxoglutarate_transaminase | |
| 95 | NAD | 318 | NADH-dependent_oxidoreductase | 99.4 |
| 96 | putative_membrane_protein | 317 | GGDEF_domain-containing_protein | 99.5 |
| 97 | cyclic_beta_1-2_glucan_synthetase | 316 | glycosyl_transferase | 99.4 |
| 98 | thioesterase | 315 | thioesterase | 99.2 |
| 100 | inosose_dehydratase | 314 | sugar_phosphate_isomerase_epimerase | 99.1 |
| 101 | hypothetical_protein | 313 | DUF423_domain-containing_protein | 100.0 |
| 102 | putative_cellulose_14-beta-cellobiosidase | 312 | cellulose_14-beta-cellobiosidase | 99.6 |
| 103 | ferritin_heavy_subunit | 311 | ferritin | 100.0 |
| 104 | putative_N-acetyltransferase_ytml | 310 | GNAT_family_N-acetyltransferase | 100.0 |
| 105 | acylaminoacyl-peptidase | 309 | S9_family_peptidase | 99.7 |
| 107 | acetate-CoA_ligase | 307 | acyl--CoA_ligase | 98.7 |
| 108 | aminopeptidase_AmpS | 306 | aminopeptidase | 99.3 |
| 109 | HTH-type_transcriptional_regulator_Betl | 305 | TetR_AcrR_family_transcriptional_regulator | 98.5 |
| 110 | hypothetical_protein | 304 | HXXEE_domain-containing_protein | 98.1 |
| 111 | hypothetical_protein | 303 | DUF4309_domain-containing_protein | 91.7 |
| 112 | HTH-type_transcriptional_regulator_betl | 302 | TetR_AcrR_family_transcriptional_regulator | 99.4 |
| 113 | hypothetical_protein | 301 | sulfurtransferase_TusA_family_protein | 97.4 |
| 114 | putative_protein_yrkE | 300 | DsrE_DsrF_DrsH-like_family_protein | 98.1 |
| 115 | copper-sensing_transcriptional_repressor_csoR | 299 | metal-sensitive_transcriptional_regulator | 100.0 |
| 116 | hypothetical_protein | 298 | DUF421_domain-containing_protein | 99.6 |
| 117 | spore_germination_protein_yndE | 297 | GerAB_ArcD_ProY_family_transporter | 100.0 |
| 118 | spore_germination_protein_KC_Flags_Precursor | 296 | GerC_family_spore_germination_protein | 100.0 |
| 119 | spore_germination_protein_KA | 295 | spore_germination_protein | 99.8 |
| 120 | cystathionine_beta-lyase | 294 | aminotransferase_class_I_II-fold | 99.0 |
| 122 | proline_iminopeptidase | 293 | alpha_beta_hydrolase | 98.0 |
| 124 | hydrolase_haloacid_{—} | 292 | YjjG_family_noncanonical_{—} | 98.8 |
| | dehalogenase_like_family | | pyrimidine_nucleotidase | |
| 125 | alpha-galactosidase | 291 | alpha-galactosidase | 98.9 |
| 126 | HTH-type_{—} | 290 | substrate-binding_{—} | 99.4 |
| | transcriptional_repressor | | domain-containing_protein | |
| 127 | two-component_sensor_kinase_YesM | 289 | histidine_kinase | 98.7 |
| 128 | putative_transcriptional_{—} | 288 | response_regulator | 97.3 |
| | regulatory_protein_yesN | | | |
| 129 | ABC_transporter_extracellular- | 287 | extracellular_solute- | 97.7 |
| | binding_protein_yurO_Flags | | binding_protein | |
| 130 | sn-glycerol-3-phosphate_{—} | 286 | sugar_ABC_{—} | 98.9 |
| | transport_system_permease_protein_ugpA | | transporter_permease | |
| 132 | sn-glycerol-3-phosphate_{—} | 285 | carbohydrate_ABC_{—} | 100.0 |
| | transport_system_permease_protein_ugpE | | transporter_permease | |
| 133 | alpha-amylase | 284 | DUF3459_domain-containing_protein | 98.3 |
| 134 | inositol_2-dehydrogenase_D- | 283 | Gfo_Idh_MocA_{—} | 99.4 |
| | chiroinositol_3-dehydrogenase | | family_oxidore-ductase | |
| 135 | putative_protein_YvdQ | 282 | DUF3231_family_protein | 100.0 |
| 136 | hypothetical_protein | 281 | WGxxGxxG-CTERM_domain-containing_protein | 100.0 |
| 137 | marR_family_transcriptional_regulator | 280 | MarR_family_transcriptional_regulator | 98.6 |
| 138 | NADH_flavin_oxidoreductase_NADH_oxidase | 279 | NADH_flavin_oxidoreductase_NADH_oxidase | 100.0 |
| 139 | araC_family_transcriptional_regulator | 278 | AraC_family_transcriptional_regulator | 98.6 |
| 140 | reductase | 277 | SDR_family_oxidoreductase | 98.9 |
| 141 | putative_MFS-type_transporter_ywoD | 276 | MFS_transporter | 99.8 |
| 142 | hydrogen_peroxide-inducible_gene_activator | 275 | LysR_family_transcriptional_regulator | 99.7 |
| 143 | tetracycline_resistance_protein | 274 | tetracycline_resistance_MFS_efflux_pump | 99.3 |
| 144 | Lacl_family_transcriptional_regulator | 273 | Lacl_family_DNA-binding_transcriptional_regulator | 99.7 |
| 145 | cellulase | 272 | glycoside_hydrolase_family_5_protein | 99.6 |
| 146 | aldo-keto_reductase_family_1_member_C1 | 27 | aldo_keto_reductase | 98.3 |
| 147 | Msm_operon_regulatory_protein | 270 | AraC_family_transcriptional_regulator | 97.6 |
| 148 | putative_tRNA-dihydrouridine_synthase | 269 | xenobiotic_reductase_b | 92.6 |
| 149 | putative_HTH-type_transcriptional_regulatorYisV | 268 | PLP-dependent_aminotransferase_family_protein | 98.6 |
| 150 | D-alanine-D-alanine_ligase | 267 | D-alanine--D-alanine_ligase | 100.0 |
| 151 | putative_streptothricin_acetyltransferase | 266 | GNAT_family_N-acetyltransferase | 98.4 |
| 152 | hypothetical_protein | 265 | DUF1349_domain-containing_protein | 100.0 |
| 153 | phosphoglycolate_phosphatase | 264 | HAD_family_phosphatase | 99.2 |
| 154 | alcohol_dehydrogenase_{—} | 263 | 2-amino-2-deoxy-D- | 98.0 |
| | zinc-containing | | mannitol_dehydrogenase_GutB1 | |
| 155 | 4-aminobutyrate_aminotransferase | 262 | aspartate_aminotransferase_family_protein | 98.8 |
| 156 | sodium-dependent_glucose_transporter_1 | 261 | MFS_transporter | 99.0 |
| 157 | fructuronate_reductase | 260 | mannitol_dehydrogenase_family_protein | 100.0 |
| 158 | mannonate_dehydratase | 259 | mannonate_dehydratase | 99.7 |
| 159 | putative_HTH-type_transcriptional_regulator_YdfH | 258 | GntR_family_transcriptional_regulator | 99.5 |
| 161 | D-mannonate_oxidoreductase | 257 | SDR_family_oxidoreductase | 99.3 |
| 162 | HTH-type_transcriptional_activator_rhaR | 256 | AraC_family_transcriptional_regulator | 98.9 |
| 163 | hypothetical_protein | 255 | glycosyl_hydrolase_115_family_protein | 99.0 |
| 164 | dihydrodipicolinate_synthase | 254 | dihydrodipicolinate_synthase_family_protein | 98.3 |
| 165 | putative_protein_yyaS | 253 | YitT_family protein | 99.1 |
| 166 | transcriptional_{—} | 252 | winged_helix-turn-helix_{—} | 98.7 |
| | regulator_slyA | | transcriptional_regulator | |
| 167 | putative_N-acetyltransferase | 251 | GNAT_family_N-acetyltransferase | 99.4 |
| 169 | amidase | 250 | amidase | 98.9 |
| 170 | hypothetical_protein | 249 | hypothetical_protein | 91.2 |
| 174 | deoxycytidine_triphosphate_{—} | 248 | deoxycytidine_{—} | 97.1 |
| | deaminase_dCTP_deaminase | | triphosphate_deaminase | |
| 175 | 30S_ribosomal_protein_S14 | 247 | 30S_ribosomal_protein_S14 | 98.9 |
| 176 | putative_GTP-binding_protein_yjiA | 246 | GTP-binding_protein | 97.9 |
| 177 | putative_cyclohydrolase | 245 | GTP_cyclohydrolase_I_FolE2 | 98.5 |
| 178 | putative_HTH-type_{—} | 244 | winged_helix-turn-helix_{—} | 100.0 |
| | transcriptional_regulator_YdgJ | | transcriptional_regulator | |
| 180 | lipid_A_export_ATP- | 242 | ABC_transporter_{—} | 98.5 |
| | binding_permease_protein_MsbA | | ATP-binding_protein | |
| 181 | lipid_A_export_ATP- | 241 | ABC_transporter_{—} | 98.2 |
| | binding_permease_protein_MsbA | | ATP-binding_protein | |

If the parent strain is Paenibacillus polymyxa strain LU17007 deposited as DSM 26970 or a similar strain having an ANI-Value of, in the order of rising preference, at least 90%, 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of Paenibacillus polymyxa strain LU17007 deposited as DSM 26970, the deleted fragment may further comprise one or more open reading frames, which encode polypeptides which have in the order of rising preference, at least 90%, 91%, 92%, 93%, 94%, 95%, 96%, 97%, 98%, 99%, or 100% sequence identity to the amino acid sequences of one or more of SEQ ID Nos: 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215. If the parent strain is Paenibacillus polymyxa strain S1B3-1 or similar strains having an ANI-Value of, in the order of rising preference, at least 90%, 91%, 92%, 93%, 94%, 95%, 96%, 97%, 98%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of Paenibacillus polymyxa strain S1B3-1, the deleted fragment may further comprise one or more open reading frames, which encode polypeptides which have in the order of rising preference, at least 90%, 91%, 92%, 93%, 94%, 95%, 96%, 97%, 98%, 99%, or 100% sequence identity to one or more of the amino acid sequences of SEQ ID NOs: 334, 335, 336, 337, 338, 339, 340, 341, 342, 343, 344, 345, 346.

In one embodiment the Paenibacillus polymyxa strain is derived from the parent strain P. polymyxa LU17007 deposited as DSM 26970, and is preferably one of the strains of the group consisting of LU20754 deposited as DSM 33970, LU21132 deposited as DSM 33971, LU21514 deposited as DSM 33972, LU21552 deposited as DSM 33973, LU22377 deposited as DSM 33974, LU22378 deposited as DSM 33975, LU22379 deposited as DSM 33976, and LU22381 deposited as DSM 33977.

Paenibacillus strains usually have the capacity to produce several different compounds which have the capacity to inhibit the growth of other Gram-positive or Gram-negative bacteria or which even inhibit Gram-positive and Gram-negative bacteria. These compounds include compounds of several classes including DAB comprising compounds, like polymyxins, tridecaptins, polypeptins, paenilipoheptins, octapeptins, jolipeptin, gavaserin or saltavalin, but also ribosomally synthesized and post-translationally modified peptides (RiPPs), like lanthionine or methyllanthionine-containing lanthipeptides, see Baindara et al., 2019, Whole genome mining reveals a diverse repertoire of lanthionine synthetases and lanthipeptides among the genus Paenibacillus, Journal of Applied Microbiology 128, 473-490,

Paenibacillus species strains lacking a functional diaminobutyrate-2-oxoglutarate-transaminase are unable to produce diaminobutyrate-2-oxoglutarate-transaminase and are unable to produce DAB comprising compounds, even, if they comprise functional gene clusters for the respective DAB comprising compound in their genomes. Based on this, they lack the detrimental effects on other Gram-positive and Gram-negative bacteria provided by the DAB comprising compounds produced under normal circumstances but remain resistant to the DAB comprising compounds they used to produce before they lost the capacity to produce a functional diaminobutyrate-2-oxoglutarate-transaminase.

This makes these Paenibacillus species strains especially suited to be used in mixtures with other bacteria, which would normally suffer from the production of DAB comprising compounds.

Accordingly, one embodiment of the invention comprises a mixture comprising a) a pure culture of a Paenibacillus species strain which does not produce DAB comprising compounds and b) a pure culture of a microorganism which is sensitive to at least one DAB comprising compound. A microorganism of b) is sensitive to a DAB comprising compound, if its growth is reduced in presence of the respective DAB comprising compound in question.

Reduced growth of a microorganism of b) can be tested via a plate assay similar to the one used in the inhibition assay described in Example 5. The plate assay comprises preferably a Paenibacillus species strain producing DAB comprising compounds, e.g. polymyxin or tridecaption or producing polymyxin and tridecaptin, which is preferably the parent strain to the Paenibacillus species strain of a) or a Paenibacillus species strain having an ANI-Value of, in the order of rising preference, at least 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% to the Paenibacillus species strain of a). A further method is to compare the growth rate of the microorganism of b) in a liquid culture comprising at least one DAB comprising compound with the growth rate of the microorganism of b) in a liquid culture not comprising the respective DAB comprising compound grown under the same conditions.

Preferably the Paenibacillus species strain of a) does not produce polymyxins or tridecaptins or does not produce polymyxins or tridecaptins and the microorganism of b) is sensitive to at least one polymyxin or at least one tridecaptin or to at least one polymyxin and at least one tridecaptin. Preferably, the Paenibacillus species strain of a) does not produce colistin or tridecaptin M or does not produce both of them, and the microorganism of b) is sensitive to colistin or to tridecaptin M or is sensitive to both of them.

The phrase “does not produce” means that the respective Paenibacillus species strain produces less than, in rising order of preference, 0.5, 0,3, 0,2, 0.1 mg/ml, even more preferred less than, in rising order of preference, 8 μg/ml, 6 μg/ml, 4 μg/ml, 2 μg/ml of colistin or tridecaptin M or both, or more preferred of any polymyxin or any tridecaptin or any polymyxin or tridecaptin or even more preferred of any DAB comprising compound. The production level is preferably measured after a growth of the Paenibacillus species strain in a soy based culture broth or similar culture broths based on yeast extract, casein or peptone, preferably it is a soy based medium, for at least 48 hours, preferably reaching a sporulation rate higher than 80%. The growth temperature is preferably between 28° C. to 37° C., more preferred 30° C. to 35° C. and even more preferred between 32° C. and 34° C. The pH-value of the culture broth is preferably between pH 6 and pH 7.

Preferably, the microorganism of b) is not sensitive to the presence of the Paenibacillus species strain of a), but is sensitive to the presence of a Paenibacillus species strain having an ANI-Value of, in the order of rising preference, at least 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% to the Paenibacillus species strain of a) and preferably comprising an open reading frame for a diaminobutyrate-2-oxoglutarate-transaminase comprising an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 217.

Preferably, the microorganism of b) is not sensitive to the presence of the Paenibacillus species strain of a) but is sensitive to the presence of the parent strain of the Paenibacillus species strain of a).

A microorganism of b) is sensitive to the presence of the parent strain of the Paenibacillus species strain of a), if it shows a reduced or no growth in the presence of the parent strain of the Paenibacillus species strain of a) in comparison to growth when grown under the same conditions and inoculum strength but in the presence of the Paenibacillus species strain of a).

In case the parent strain of the Paenibacillus species strain of a) is not available the parent strain of the Paenibacillus species strain of a) can be replaced by a Paenibacillus species strain having an ANI-Value of, in the order of rising preference, at least 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% to the Paenibacillus species strain of a) and preferably comprising an open reading frame for a diaminobutyrate-2-oxoglutarate-transaminase comprising an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 217.

The microorganism of b) can belong to any fungal or bacterial species, preferably it belongs to a Gram-positive or Gram-negative bacterial species.

In a preferred embodiment the microorganism of b) is selected from Azotobacter, Azospirillum, Pseudomonas, Burkholderia, Paraburkholderia, Rhizobium, Bradyrhizobium, Sinorhizobium, Mesorhizobium, Bacillus or Streptomyces.

Preferred species of the genus Rhizobium are:

Rhizobium phaseoli, preferably the strain DSM30137 (also known as strain ATCC 14482), Rhizobium radiobacter (formerly Agrobacterium radiobacter), preferably the strains DSM30205 or K1026.

In one embodiment the microorganism of b) is selected from Rhizobium strains having an ANI-Value of, in the order of rising preference, at least 95%, 96%, 97%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% to the genome of Rhizobium phaseoli strain ATCC 14482 (GenBank AccNo: RJJV00000000.1), Rhizobium leguminosarum bv. viceae strain 3841 (GenBank AccNo: AM236080.1), Rhizobium leguminosarum bv. viceae strain 1435 or Rhizobium leguminosarum bv. phaseoli strain FA23 (GenBank AccNo: ATTN00000000).

Preferred species of the genus Bradyrhizobium are:

Bradyrhizobium japonicum, preferably the strains 532C, USDA 110, USDA 122, USDA 135 and USDA 4.

In one embodiment the microorganism of b) is selected from Bradyrhizobium strains having an ANI-Value of, in the order of rising preference, at least 95%, 96%, 97%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% to the genome of B. japonicums strain 532C, B. japonicums strain USDA 110, (GenBank AccNo: BA000040), B. japonicums strain USDA 122, (GenBank AccNo: AXAX00000000), B. japonicums strain USDA 135 (GenBank AccNo: AXAT00000000), more B. japonicums strain USDA 4 (GenBank AccNo: AXAF00000000).

Preferred species of the genus Paraburkholderia are:

Paraburkholderia phytofirmans, preferably the strains DSM17436 and PsJN.

In one embodiment the microorganism of b) is selected from Paraburkholderia strains having an ANI-Value of, in the order of rising preference, at least 95%, 96%, 97%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% to the genome of P. phytofirmans strain PsJN (GenBank AccNo: AAUH01000000).

Preferred species of the genus Pseudomonas are:

Pseudomonas koreensis, preferably the strains (IA-250_TP_15), Pseudomonas chlororaphis, preferably the strains MA342 and AFS009.

In one embodiment the microorganism of b) is selected from Pseudomonas strains having an ANI-Value of, in the order of rising preference, at least 95%, 96%, 97%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% to the genome of at least one of P. koreensis strain: D26 (GenBank AccNo: CP014947), P. koreensis strain C112 (GenBank AccNo: MPLD00000000), P. koreensis strain CRS05-R5 (GenBank AccNo: CP015852), P. chlororaphis strain O6 (GenBank AccNo: CM001490.1), P. chlororaphis strain HT66 (GenBank AccNo: ATBG00000000), or P. chlororaphis strain UFB2 (GenBank AccNo: CP011020).

Preferred species of the genus Bacillus are:
Bacillus amyloliquefaciens, preferably the strains 71 (NRRL B-67021), PTA-4838, F727, D747, ENV503, FZB24, FZB42, H2 and RT1301,
Bacillus paralicheniformis, preferably the strain RT184,
Bacillus licheniformis, preferably the strains ATCC 14580 or FMCH001,
Bacillus subtilis, preferably the strains FMCH002, RT1477, AB/BS03, HAI-0404, QST713, Y1336, IAB/BS03 and BU1814,
Bacillus mycoides, preferably the strain BMJ,
Bacillus firmus, preferably the strains NCIM 2637 and I-1582,
Bacillus pumilus, preferably the strains QST2808 and F33 (syn. INR7, AP18, F-22, BU 1433),
Bacillus simplex, preferably the strain ABU-288,
Bacillus sp., preferably the strain ITB105,
Bacillus thuringiensis subsp. kurstaki, preferably the strains EG 2348, EVB-113-19, ABTS 351, PB 54, SA11, SA12 and EG 2348,
Bacillus thuringiensis subsp. aizawai, preferably the strains ABTS-1857 and GC-91,
Bacillus thuringiensis subsp. israeliensis, preferably the strain AM65-52, and Bacillus velezensis preferably the strain MBI600. Some of the strains listed above are also known under different species names, e.g. strains MBI600 and QST713 had been allocated to the species Bacillus subtilis, to the species Bacillus velezensis and the species Bacillus amyloliquefaciens.

In one embodiment the microorganism of b) is selected from Bacillus strains having an ANI-Value of, in the order of rising preference, at least 95%, 96%, 97%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% to the genome of at least one of B. velezensis strain QST713 (GenBank AccNo: CP025079), B. amyloliquefaciens FZB42 (GenBank AccNo: CP000560), B. amyloliquefaciens GB03 (GenBank AccNo: AYTJ00000000), B. velezensis MBI600 (GenBank AccNo: CP033205.1) or Bacillus licheniformis ATCC 14580 (GenBank AccNo: CP034569), preferably to the genome of B. velezensis MBI600 or Bacillus licheniformis ATCC 14580.

Preferred species of the genus Streptomyces are:

Streptomyces griseoviridis, preferably the strain K61, and Streptomyces lydicus, preferably the strain WYEC 108.

In one embodiment the microorganism of b) is selected from Streptomyces strains having an ANI-Value of, in the order of rising preference, at least 95%, 96%, 97%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% to the genome of at least one of Str. griseoviridis strain K61 (GenBank AccNo: CP029078), Str. griseoviridis strain F1-27 (GenBank AccNo: CP034687), Str. lydicus strain WYEC 108 (GenBank AccNo: CP029042) or Str. lydicus strain ATCC 25470 (GenBank AccNo: RDTD01000000), preferably to Str. lydicus strain WYEC 108.

Preferably the Rhizobium, Bradyrhizobium, Paraburkholderia, Pseudomonas, Bacillus, Streptomyces strains are not sensitive to the presence Paenibacillus species strain of a) but are sensitive to the presence of the parent strain of the Paenibacillus species strain of a) or a Paenibacillus species strain having an ANI-Value of, in the order of rising preference, at least 95%, 96%, 97%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% to the Paenibacillus species strain of a) and preferably comprising an open reading frame for a diaminobutyrate-2-oxoglutarate-transaminase comprising an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 217.

Preferably the microorganism of b) is selected from the species Rhizobium phaseoli Rhizobium radiobacter, Bradyrhizobium japonicum Pseudomonas koreensis, Paraburkholderia phytofirmans, Bacillus licheniformis, Streptomyces lydicus, Bacillus subtilis or Bacillus velezensis.

In one embodiment the microorganism of b) is selected from the strains:

Rhizobium phaseoli strain DSM30137, Rhizobium radiobacter strain DSM30205, Bradyrhizobium japonicum strain 532C, Pseudomonas koreensis strain (IA-250_TP_15), Paraburkholderia phytofirmans strain DSM17436, Bacillus licheniformis strain DSM13, Streptomyces lydicus strain WYEC 108, Bacillus subtilis strain BU1814, Bacillus velezensis strain MBI600.

In one embodiment the Paenibacillus species strain of a) does not comprise a functional diaminobutyrate-2-oxoglutarate-transaminase, preferably does not comprise a functional diaminobutyrate-2-oxoglutarate-transaminase comprising an amino acid sequence of, in the order of rising preference, at least 90%, 91%, 92%, 93%, 94%, 95%, 96%, 97%, 98%, 99%, or 100% sequence identity to at least one of SEQ ID NO: 217, 218, 219, 220 or 221.

In one embodiment the Paenibacillus species strain of a) does not produce colistin and the microorganism of b) is sensitive to colistin,
more preferred the Paenibacillus species strain of a) does not produce a polymyxin and the microorganism of b) is sensitive to at least one polymyxin.

In one embodiment the Paenibacillus species strain of a) does not produce tridecaptin M, and the microorganism of b) is sensitive to tridecaptin M,
preferably the Paenibacillus species strain of a) does not produce tridecaptin A, B, or M and the microorganism of b) is sensitive to tridecaptin A, B, and M.
more preferred the Paenibacillus species strain of a) does not produce any tridecaptin and the microorganism of b) is sensitive to at least one tridecaptin.

In one embodiment the Paenibacillus species strain of a) does not produce tridecaptin M and colistin, and the microorganism of b) is sensitive to at least one of tridecaptin M or colistin, preferably the Paenibacillus species strain of a) does not produce tridecaptin A, B, M and colistin and the microorganism of b) is sensitive to at least one of tridecaptin A, B, M and colistin, more preferred the Paenibacillus species strain of a) does not produce any tridecaptin and does not produce any polymyxin and the microorganism of b) is sensitive to at least one tridecaptin or at least one polymyxin or at least one tridecaptin and at least one polymyxin.

In another embodiment the Paenibacillus species strain of a) does not comprise an open reading frame for a diaminobutyrate-2-oxoglutarate-transaminase comprising an amino acid sequence of, in the order of rising preference, at least 90%, 91%, 92%, 93%, 94%, 95%, 96%, 97%, 98%, 99%, or 100% sequence identity to at least one of SEQ ID NO: 217, 218, 219, 220 or 221.

In another embodiment the Paenibacillus species strain of a) does not comprise an open reading frame for a diaminobutyrate-2-oxoglutarate-transaminase comprising an amino acid sequence of, in the order of rising preference, at least 90%, 91%, 92%, 93%, 94%, 95%, 96%, 97%, 98%, 99%, or 100% sequence identity to at least one of SEQ ID NO: 217, 218, 219, 220 or 221 and does not comprise a polynucleotide sequence encoding an amino acid sequence of at least 90% sequence identity to SEQ ID NO: 1.

In one embodiment the Paenibacillus species strain of a) does comprise an open reading frame for a diaminobutyrate-2-oxoglutarate-transaminase comprising an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to at least one of SEQ ID NO: 217, 218, 219, 220 or 221, but comprises a polynucleotide sequence encoding an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 1, but does not produce polymyxin or tridecaptin or does not produce polymyxin and does not produce tricecaptin.

In another embodiment the Paenibacillus species strain of a) does not comprise an open reading frame for a diaminobutyrate-2-oxoglutarate-transaminase comprising an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to at least one of SEQ ID NO: 217, 218, 219, 220 or 221, but comprises a polynucleotide sequence encoding an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 1.

In another embodiment the Paenibacillus species strain of a) does not comprise an open reading frame for a diaminobutyrate-2-oxoglutarate-transaminase comprising an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to at least one of SEQ ID NO: 217, 218, 219, 220 or 221, but comprises a polynucleotide sequence encoding an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 1, but does not produce tridecaptin A, B, or M, preferably does not produce tridecaptins.

In another embodiment the Paenibacillus species strain of a) does not comprise an open reading frame for a diaminobutyrate-2-oxoglutarate-transaminase comprising an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to at least one of SEQ ID NO: 217, 218, 219, 220 or 221, but comprises a polynucleotide sequence encoding an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 1, but does not produce tridecaptins, preferably does not produce tridecaptin A, B, or M and does not produce colistin, preferably does not produce polymyxins.

In another embodiment the Paenibacillus species strain of a) does not comprise an open reading frame for a diaminobutyrate-2-oxoglutarate-transaminase comprising an amino acid sequence of, in the order of rising preference, at least 90%, 91%, 92%, 93%, 94%, 95%, 96%, 97%, 98%, 99%, or 100% sequence identity to at least one of SEQ ID NO: 217, 218, 219, 220 or 221 and the microorganism of b) is not sensitive to the presence of the Paenibacillus species strain of a), but is sensitive to the presence of the parent strain of the Paenibacillus species strain of a).

In another embodiment the Paenibacillus species strain of a) does not comprise an open reading frame for a diaminobutyrate-2-oxoglutarate-transaminase comprising an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to at least one of SEQ ID NO: 217, 218, 219, 220 or 221 and the microorganism of b) is sensitive to at least one of polymyxin, tridecaptin, polypeptin, paenilipoheptin, octapeptin, jolipeptin, gavaserin or saltavalin and is sensitive to the presence of the parent strain of the Paenibacillus species strain of a).

In another embodiment the Paenibacillus species strain of a) comprises no or only one locus encoding an amino acid sequence comprising, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 1 and does not produce polymyxin or tridecaptin. Preferably, the parent strain of the Paenibacillus species strain of a) comprises at least one locus encoding an amino acid sequence comprising, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 1 and does produce at least one tridecaptin or does preferably produce at least one polymyxin and at least one tridecaptin.

In a further embodiment, the Paenibacillus species strain of a) comprises only one genetic locus comprising, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a polynucleotide sequence of any one of SEQ IN NOs: 222, 223, 224, 225, 413, 414, 415, 416, 417, 418 and does not comprise an open reading frame for a diaminobutyrate-2-oxoglutarate-transaminase comprising an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 217. Preferably, the parent strain of the Paenibacillus species strain of a) comprises at least one genetic locus comprising, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a polynucleotide sequence of any one of SEQ IN NOs: 222, 223, 224, 225 and does comprise an open reading frame for a diaminobutyrate-2-oxoglutarate-transaminase comprising an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 217.

In a further embodiment, the Paenibacillus species strain of a) does not comprise a polynucleotide sequence encoding an amino acid sequence comprising, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 1. Preferably, the parent strain of the Paenibacillus species strain of a) comprises at least two polynucleotide sequence encoding an amino acid sequence comprising, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 1, wherein the two polynucleotides sequences comprise at least, in the order of rising preference, 90%, 91%, 92%, 93%, 94%, 95%, 96%, 97%, 98%, 99%, or 100% sequence identity to each other.

In a further embodiment, the Paenibacillus species strain of a) does not comprise a continuous genome fragment of, in the order of rising preference, less than 350, 340, 330, 320, 310 or 300 kilobases, which comprises a plurality of, preferably at least 147, open reading frames encoding polypeptide sequences of, in the order of rising preference, at least 90%, 91%, 92%, 93%, 94%, 95%, 96%, 97%, 98%, 99%, or 100% sequence identity
a) to the amino acid sequences of SEQ ID NOs: 2, 3, 5, 7, 8, 9, 10, 11, 12, 13, 14, 15, 16, 17, 18, 19, 20, 21, 22, 23, 25, 27, 28, 30, 31, 32, 34, 36, 37, 38, 39, 40, 41, 44, 45, 47, 48, 50, 51, 52, 53, 54, 55, 58, 60, 61, 62, 63, 64, 65, 66, 67, 68, 69, 70, 72, 77, 78, 79, 80, 81, 82, 83, 84, 86, 87, 89, 91, 92, 93, 94, 95, 96, 97, 98, 100, 101, 102, 103, 104, 105, 107, 108, 109, 110, 111, 112, 113, 114, 115, 116, 117, 118, 119, 120, 122, 124, 125, 126, 127, 128, 129, 130, 132, 133, 134, 135, 136, 137, 138, 139, 140, 141, 142, 143, 144, 145, 146, 147, 148, 149, 150, 151, 152, 153, 154, 155, 156, 157, 158, 159, 161, 162, 163, 164, 165, 166, 167, 169, 170, 174, 175, 176, 177, 178, 180, 181, or
b) to the amino acid sequences of SEQ ID NOs: 241, 242, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 309, 310, 311, 312, 313, 314, 315, 316, 317, 318, 319, 320, 321, 322, 323, 324, 325, 326, 327, 328, 329, 330, 331, 332, 333, 347, 348, 349, 350, 351, 352, 353, 354, 355, 356, 357, 358, 360, 363, 363, 364, 365, 366, 367, 369, 370, 371, 372, 373, 374, 375, 376, 377, 378, 379, 380, 381, 382, 383, 384, 385, 387, 388, 389, 390, 391, 392, 393, 394, 395, 396, 397, 398, 399, 400, 401, 402, 403, 404, 405, 406, 407.

Preferably the genomic fragment lost due to the recombination event comprises at least one coding sequence of, in the order of rising preference, at least 90%, 91%, 92%, 93%, 94%, 95%, 96%, 97%, 98%, 99%, or 100% sequence identity for each one of the amino acid sequences listed above under a) or b), respectively.

Preferably, the parent strain of the Paenibacillus species strain of a) comprises a continuous genome fragment of, in the order of rising preference, less than 350, 340, 330, 320, 310 or 300 kilobases, which comprises at least 147 open reading frames encoding polypeptide sequences of at least 95% sequence identity
a) to the amino acid sequences of SEQ ID NOs: 2, 3, 5, 7, 8, 9, 10, 11, 12, 13, 14, 15, 16, 17, 18, 19, 20, 21, 22, 23, 25, 27, 28, 30, 31, 32, 34, 36, 37, 38, 39, 40, 41, 44, 45, 47, 48, 50, 51, 52, 53, 54, 55, 58, 60, 61, 62, 63, 64, 65, 66, 67, 68, 69, 70, 72, 77, 78, 79, 80, 81, 82, 83, 84, 86, 87, 89, 91, 92, 93, 94, 95, 96, 97, 98, 100, 101, 102, 103, 104, 105, 107, 108, 109, 110, 111, 112, 113, 114, 115, 116, 117, 118, 119, 120, 122, 124, 125, 126, 127, 128, 129, 130, 132, 133, 134, 135, 136, 137, 138, 139, 140, 141, 142, 143, 144, 145, 146, 147, 148, 149, 150, 151, 152, 153, 154, 155, 156, 157, 158, 159, 161, 162, 163, 164, 165, 166, 167, 169, 170, 174, 175, 176, 177, 178, 180, 181, or
b) to the amino acid sequences of SEQ ID NOs: 241, 242, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 309, 310, 311, 312, 313, 314, 315, 316, 317, 318, 319, 320, 321, 322, 323, 324, 325, 326, 327, 328, 329, 330, 331, 332, 333, 347, 348, 349, 350, 351, 352, 353, 354, 355, 356, 357, 358, 360, 363, 363, 364, 365, 366, 367, 369, 370, 371, 372, 373, 374, 375, 376, 377, 378, 379, 380, 381, 382, 383, 384, 385, 387, 388, 389, 390, 391, 392, 393, 394, 395, 396, 397, 398, 399, 400, 401, 402, 403, 404, 405, 406, 407.

Preferably the genomic fragment lost due to the recombination event comprises at least one coding sequence of, in the order of rising preference, at least 90%, 91%, 92%, 93%, 94%, 95%, 96%, 97%, 98%, 99%, or 100% sequence identity for each one of the amino acid sequences listed above under a) or b), respectively.

In a further embodiment, the Paenibacillus species strain of a), and/or the parent strain of, the Paenibacillus species strain of a), has an ANI-Value of at least 90%, 91%, 92%, 93%, 94%, 95%, 96%, 97%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% to the genome of at least one strain selected from the group comprising: P. polymyxa LU17007, P. polymyxa M152, P. polymyxa SQR-21, P. polymyxa M1, P. polymyxa SC2, P. polymyxa HY96-2, P. polymyxa YC0136, P. polymyxa E681, P. polymyxa CR1, P. polymyxa J, P. polymyxa ZF197, P. polymyxa WLY78, P. ottowii strain MS2379, P. peoriae ZF390, P. peoriae HS311, P. kribbensis AM49, P. kribbensis PS04, P. terrae HPL-003, P. sp. Aloe-11.

In a further embodiment, the Paenibacillus species strain of a), and/or the parent strain of, the Paenibacillus species strain of a), has an ANI-Value of, in the order of rising preference, at least 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. polymyxa LU17007 deposited as DSM 26970.

In a further embodiment, the Paenibacillus species strain of a), and/or the parent strain of, the Paenibacillus species strain of a), has an ANI-Value of, in the order of rising preference, at least 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9%, to the genome of P. polymyxa SQR-21.

It has been observed that the Paenibacillus species strains of the invention produces a modified spectrum of fusaricidins compared to their parent strains, in which fusaricidin compounds comprising glutamine have a higher concentration than the respective fusaricidin compound comprising asparagine at the respective position of the molecule. Without wishing to be bound to a particular hypothesis, this phenomenon could be due to a loss of the diaminobutyrate-2-oxoglutarate-transaminase, which would otherwise create a sink of glutamate by producing DAB, thereby enhancing the pool of glutamine available for the production of glutamine comprising fusaricidin compounds.

Accordingly, another part of the invention is a Paenibacillus species strain producing more of at least one glutamine comprising fusaricidin, paeniprolixin or paeniserine than of its respective asparagine comprising counterpart.

A glutamine comprising counterpart of a given fusaricidin is a fusaricidin comprising the same structure in its lipid part and comprising the same sequence of amino acid building blocks in its peptide part except for one amino acid in which the asparagine is exchanged for glutamine, for example, fusaricidin A and fusaricidin B are considered counterparts.

Preferably, the Paenibacillus species strain produces
a) more fusaricidin LiF04b (Fus B) than fusaricidin LiF04a (Fus A), or
b) more fusaricidin LiF03b than fusaricidin LiF03a, or
c) more fusaricidin LiF05b than fusaricidin LiF05a, or
d) more fusaricidin LiF06b than fusaricidin LiF06a, or
e) more fusaricidin LiF07b than fusaricidin LiF07a, or
f) any combination of a), b), c), d) and e).

In one embodiment the Paenibacillus species strain produces more fusaricidin LiF04b (Fus B) than fusaricidin LiF04a (Fus A).

Preferably the Paenibacillus species strain producing more of at least one glutamine comprising fusaricidin, paeniprolixin or paeniserine than of its respective asparagine comprising counterpart in comparison to a Paenibacillus species strain having an ANI-Value of, in the order of rising preference, at least 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% to the Paenibacillus species strain, and preferably comprising an open reading frame for a diaminobutyrate-2-oxoglutarate-transaminase comprising an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 217, preferably the comparison is performed with the parent Paenibacillus species strain.

In another embodiment the Paenibacillus species strain producing more of at least one glutamine comprising fusaricidin, paeniprolixin or paeniserine than of its respective asparagine comprising counterpart does not comprise an open reading frame for a diaminobutyrate-2-oxoglutarate-transaminase comprising an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 217.

In another embodiment the Paenibacillus species strain producing more of at least one glutamine comprising fusaricidin, paeniprolixin or paeniserine than of its respective asparagine comprising counterpart comprises no or only one locus encoding an amino acid sequence comprising, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 1 and does not produce polymyxin or tridecaptin. Preferably, the parent strain of the Paenibacillus species strain of a) comprises at least one locus encoding an amino acid sequence comprising, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 1 and does produce at least one tridecaptin and does preferably produce at least one polymyxin and at least one tridecaptin.

In a further embodiment, the Paenibacillus species strain producing more of at least one glutamine comprising fusaricidin, paeniprolixin or paeniserine than of its respective asparagine comprising counterpart comprises only one genetic locus comprising, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a polynucleotide sequence of any one of SEQ IN NOs: 222, 223, 224, 225, 413, 414, 415, 416, 417, 418 and does not comprise an open reading frame for a diaminobutyrate-2-oxoglutarate-transaminase comprising an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 217. Preferably, the parent strain of the Paenibacillus species strain of a) comprises at least one genetic locus comprising, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a polynucleotide sequence of any one of SEQ IN NOs: 222, 223, 224, 225 and does comprise an open reading frame for a diaminobutyrate-2-oxoglutarate-transaminase comprising an amino acid sequence of, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 217.

In a further embodiment, the Paenibacillus species strain producing more of at least one glutamine comprising fusaricidin, paeniprolixin or paeniserine than of its respective asparagine comprising counterpart does not comprise a polynucleotide sequence encoding an amino acid sequence comprising, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 1. Preferably, the parent strain of the Paenibacillus species strain of a) comprises at least one polynucleotide sequence encoding an amino acid sequence comprising, in the order of rising preference, at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 1.

In a further embodiment, the Paenibacillus species strain producing more of at least one glutamine comprising fusaricidin, paeniprolixin or paeniserine than of its respective asparagine comprising counterpart does not comprise a continuous genome fragment of, in the order of rising preference, less than 350, 340, 330, 320, 310 or 300 kilobases, which comprises at least 147 open reading frames encoding polypeptide sequences of at least 95% sequence identity
c) to the amino acid sequences of SEQ ID NOs: 2, 3, 5, 7, 8, 9, 10, 11, 12, 13, 14, 15, 16, 17, 18, 19, 20, 21, 22, 23, 25, 27, 28, 30, 31, 32, 34, 36, 37, 38, 39, 40, 41, 44, 45, 47, 48, 50, 51, 52, 53, 54, 55, 58, 60, 61, 62, 63, 64, 65, 66, 67, 68, 69, 70, 72, 77, 78, 79, 80, 81, 82, 83, 84, 86, 87, 89, 91, 92, 93, 94, 95, 96, 97, 98, 100, 101, 102, 103, 104, 105, 107, 108, 109, 110, 111, 112, 113, 114, 115, 116, 117, 118, 119, 120, 122, 124, 125, 126, 127, 128, 129, 130, 132, 133, 134, 135, 136, 137, 138, 139, 140, 141, 142, 143, 144, 145, 146, 147, 148, 149, 150, 151, 152, 153, 154, 155, 156, 157, 158, 159, 161, 162, 163, 164, 165, 166, 167, 169, 170, 174, 175, 176, 177, 178, 180, 181, or
d) to the amino acid sequences of SEQ ID NOs: 241, 242, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 309, 310, 311, 312, 313, 314, 315, 316, 317, 318, 319, 320, 321, 322, 323, 324, 325, 326, 327, 328, 329, 330, 331, 332, 333, 347, 348, 349, 350, 351, 352, 353, 354, 355, 356, 357, 358, 360, 363, 363, 364, 365, 366, 367, 369, 370, 371, 372, 373, 374, 375, 376, 377, 378, 379, 380, 381, 382, 383, 384, 385, 387, 388, 389, 390, 391, 392, 393, 394, 395, 396, 397, 398, 399, 400, 401, 402, 403, 404, 405, 406, 407.

Preferably, the parent strain of the Paenibacillus species strain producing more of at least one glutamine comprising fusaricidin, paeniprolixin or paeniserine than of its respective asparagine comprising counterpart comprises a continuous genome fragment of, in the order of rising preference, less than 350, 340, 330, 320, 310 or 300 kilobases, which comprises at least 147 open reading frames encoding polypeptide sequences of at least 95% sequence identity
a) to the amino acid sequences of SEQ ID NOs: 2, 3, 5, 7, 8, 9, 10, 11, 12, 13, 14, 15, 16, 17, 18, 19, 20, 21, 22, 23, 25, 27, 28, 30, 31, 32, 34, 36, 37, 38, 39, 40, 41, 44, 45, 47, 48, 50, 51, 52, 53, 54, 55, 58, 60, 61, 62, 63, 64, 65, 66, 67, 68, 69, 70, 72, 77, 78, 79, 80, 81, 82, 83, 84, 86, 87, 89, 91, 92, 93, 94, 95, 96, 97, 98, 100, 101, 102, 103, 104, 105, 107, 108, 109, 110, 111, 112, 113, 114, 115, 116, 117, 118, 119, 120, 122, 124, 125, 126, 127, 128, 129, 130, 132, 133, 134, 135, 136, 137, 138, 139, 140, 141, 142, 143, 144, 145, 146, 147, 148, 149, 150, 151, 152, 153, 154, 155, 156, 157, 158, 159, 161, 162, 163, 164, 165, 166, 167, 169, 170, 174, 175, 176, 177, 178, 180, 181, or
b) to the amino acid sequences of SEQ ID NOs: 241, 242, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 309, 310, 311, 312, 313, 314, 315, 316, 317, 318, 319, 320, 321, 322, 323, 324, 325, 326, 327, 328, 329, 330, 331, 332, 333, 347, 348, 349, 350, 351, 352, 353, 354, 355, 356, 357, 358, 360, 363, 363, 364, 365, 366, 367, 369, 370, 371, 372, 373, 374, 375, 376, 377, 378, 379, 380, 381, 382, 383, 384, 385, 387, 388, 389, 390, 391, 392, 393, 394, 395, 396, 397, 398, 399, 400, 401, 402, 403, 404, 405, 406, 407.

In a further embodiment, the Paenibacillus species strain producing more of at least one glutamine comprising fusaricidin, paeniprolixin or paeniserine than of its respective asparagine comprising counterpart and/or its parent strain, has an ANI-Value of at least 90%, 91%, 92%, 93%, 94%, 95%, 96%, 97%, 98%, 99%, or 100% to the genome of at least one strain selected from the group comprising: P. polymyxa LU17007, P. polymyxa SQR-21, P. polymyxa M1, P. polymyxa SC2, P. polymyxa HY96-2, P. polymyxa YC0136, P. polymyxa E681, P. polymyxa CR1, P. polymyxa J, P. polymyxa ZF197, P. polymyxa WLY78, P. ottowii strain MS2379, P. peoriae ZF390, P. peoriae HS311, P. kribbensis AM49, P. kribbensis PS04, P. terrae HPL-003, P. sp. Aloe-11.

In a further embodiment, the Paenibacillus species strain producing more of at least one glutamine comprising fusaricidin, paeniprolixin or paeniserine than of its respective asparagine comprising counterpart and/or its parent strain has an ANI-Value of, in the order of rising preference, at least 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100.0% to the genome of P. polymyxa LU17007 deposited as DSM 26970.

In a further embodiment, the Paenibacillus species strain producing more of at least one glutamine comprising fusaricidin, paeniprolixin or paeniserine than of its respective asparagine comprising counterpart and/or its parent strain has an ANI-Value of, in the order of rising preference, at least 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or 100.0% to the genome of P. polymyxa SQR-21.

The Paenibacillus species strains of the invention and the mixtures of these Paenibacillus species strains are usually cultured in culture broth. This culture broth can then directly be dried by technologies available in the art, e.g. via spray drying, or can first be separated in a fraction comprising the spores of the Paenibacillus species strain and other solid parts of the culture medium and another fraction, comprising the liquid parts of the culture broth i.e. the cell-free culture broth, via technologies available in the art, e.g. via centrifugation or filtration, and then either used in liquid or wet form or further dried to produce a powder. All of these as well as the whole culture broth comprising the liquid components and the spores and cells of the Paenibacillus species strain, can also be extracted to produce a cell-free extract comprising secondary compounds, preferably comprising at least one fusaricidin compound.

Accordingly, another part of the invention is a whole culture broth, spores, cell-free culture broth or cell-free extract comprising a higher amount of at least one glutamine comprising fusaricidin, paeniprolixin or paeniserine compound than the amount of its respective asparagine comprising counterpart.

A further part of the invention is a method to produce a whole culture broth, spores, cell-free culture broth or cell-free extract, comprising the steps of a) providing a pure culture of a Paenibacillus species strain of the invention, b) growing it in a culture broth and c) harvesting the whole culture broth, spores or cell-free culture broth and optionally d) extracting the whole culture broth, spores or cell-free culture broth.

The whole culture broth, spores, cell-free culture broth of the Paenibacillus species strain of the invention or cell-free extracts thereof and agricultural compositions comprising whole culture broth, spores, cell-free culture broth of the Paenibacillus species strain of the invention or cell-free extracts thereof can be used in methods for controlling, suppressing or preventing fungal infection of plants comprising the steps of
a) providing a whole culture broth, spores, cell-free culture broth of the Paenibacillus species strain of the invention or a cell-free extract of at least one of these and
b) applying an effective amount of the whole culture broth, spores, cell-free culture broth of the Paenibacillus species strain of the invention or the cell-free extract to the plant pathogenic fungi, their habitat, the host plants, plant propagation material or the soil used to grow the plant.

The Paenibacillus species strains of the invention and the mixtures of these Paenibacillus species strains and a further microorganism can be used to prepare agrochemical compositions, comprising these Paenibacillus species strains or mixtures and an auxiliary.

The agricultural compositions are preferably customary types of agrochemical compositions, e.g. solutions, emulsions, suspensions, dusts, powders, pastes, granules, pressings, capsules, and mixtures thereof. Examples for composition types (see also “Catalogue of pesticide formulation types and international coding system”, Technical Monograph No. 2, 6^{th} Ed. May 2008, CropLife International) are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials, such as seeds (e.g. GF). The compositions are prepared in a known manner, such as described by Mollet and Grubemann, Formulation technology, Wiley VCH, Weinheim, 2001; or by Knowles, New developments in crop protection product formulation, Agrow Reports DS243, T&F Informa, London, 2005. The invention also relates to agrochemical compositions comprising an Paenibacillus strain of the invention and an auxiliary. Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers, and binders. Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e.g. toluene, paraffin, tetrahydronaphthalene, and alkylated naphthalenes; alcohols, e.g. ethanol, propanol, butanol, benzyl alcohol, cyclohexanol, glycols; DMSO; ketones, e.g. cyclohexanone; esters, e.g. lactates, carbonates, fatty acid esters, gamma-butyrolactone; fatty acids; phosphonates; amines; amides, e.g. N-methyl pyrrolidone, fatty acid dimethyl amides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.). Preferred agrochemical compositions for Paenibacillus strains have been described in WO2019/222253 and WO2022/023109.

Suitable anionic surfactants are alkali, alkaline earth or ammonium salts of sulfonates, sulfates, phosphates, carboxylates, and mixtures thereof. Examples of sulfonates are alkylaryl sulfonates, diphenyl sulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and of alkylnaphthalenes, sulfosuccinates, or sulfosuccinamates. Examples of sulfates are sulfates of fatty acids, of oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates. Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters, or monoglycerides. Examples of sugar-based surfactants are sorbitans, ethoxylated sorbitans, sucrose and glucose esters, or alkylpolyglucosides. Examples of polymeric surfactants are home- or copolymers of vinyl pyrrolidone, vinyl alcohols, or vinyl acetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide, and polypropylene oxide. Suitable polyelectrolytes are polyacids or polybases. Examples of polyacids are alkali salts of polyacrylic acid or polyacid comb polymers. Examples of polybases are polyvinyl amines or polyethylene amines.

Suitable adjuvants are compounds, which have a negligible or even no pesticidal activity themselves, and which improve the biological performance of the compound I on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e.g. xanthan gum, carboxymethyl cellulose), inorganic clays (organically modified or unmodified), polycarboxylates, and silicates.

Suitable bactericides are bronopol and isothiazolinone derivatives, such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinyl pyrrolidones, polyvinyl acetates, polyvinyl alcohols, polyacrylates, biological or synthetic waxes, and cellulose ethers.

The agrochemical compositions generally comprise between 0.01 and 95%, preferably between 0.1 and 90%, more preferably between 1 and 70%, and in particular between 10 and 60%, by weight of cells or spores of the Paenibacillus strain.

The amount of these cells or spores is preferably between 5% w/w and 50% w/w, 10% w/w and 50% w/w, 15% w/w and 50% w/w, 30% w/w and 50% w/w, or 40% w/w and 50% w/w, or between 5% w/w and 40% w/w, 10% w/w and 40% w/w, 15% w/w and 40% w/w, 30% w/w and 40% w/w, or between 10% w/w and 40% w/w, 15% w/w and 40% w/w, 30% w/w and 40% w/w of the agrochemical composition.

The cells or spores of the Paenibacillus strains are usually present in the form of solid particles having an average particle size of 1 to 150 μm, or in an increased order of preference of 1 to 100 μm, 1 to 75 μm, 1 to 50 μm, 1 to 25 μm, 1 to 10 μm, or 1 to 8 μm (determined according to light scattering method in liquid dispersion according to CIPAC method 187).

The density number of spores per ml can be determined by identifying the number of colony-forming units (CFU) on agar medium e.g. potato dextrose agar after incubation for several days at temperatures of about 20 to about 35° C. The amount of CFU/g of biomass used to prepare agrochemical compositions of the invention are usually between 1×10⁸ CFU/g to 1×10¹¹ CFU/g, or 1×10⁸ CFU/g to 1×10¹⁰ CFU/g, or 5×10⁸ to 5×10¹⁰ CFU/g, preferably between 1×10⁹ CFU/g to 1×10¹⁰ CFU/g. The CFU/g of biomass will influence the amount of biomass which is used to prepare the formulations of the invention. Biomass having a comparatively high amount of CFU/g can be used to prepare formulations having a comparatively low amount of biomass. The amount of biomass used for preparing the formulations of the invention is usually selected to fit the amount of CFU per hectare, which should be applied for the respective purpose.

For the purposes of treatment of plant propagation materials, particularly seeds, solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC), and gels (GF) are usually employed. The compositions in question give, after two-to-tenfold dilution, active substance concentrations of from 0.01 to 60% by weight, preferably from 0.1 to 40%, in the ready-to-use preparations. Application can be carried out before or during sowing. Methods for applying the mixtures or agrochemical compositions comprising the mixtures, respectively, onto young plants and propagation material like seedlings, rooted/unrooted cuttings, plants derived from cell-culture, include dressing, coating, pelleting, dusting, soaking, as well as in-furrow application methods. Preferably, the mixtures and agrochemical compositions thereof, respectively, are applied on to seeds by a method such that germination is not induced, e.g. by seed dressing, pelleting, coating, and dusting.

Various types of oils, wetters, adjuvants, fertilizers, or micronutrients, and further pesticides (e. g. fungicides, growth regulators, herbicides, insecticides, safeners) may be added to the mixtures or the agrochemical compositions thereof as premix, or, not until immediately prior to use (tank mix). These agents can be admixed with the mixtures or the agrochemical compositions according to the invention in a weight ratio of 1:100 to 100:1, preferably 1:10 to 10:1.

The Paenibacillus strains and the agrochemical composition comprising these strains ae usually applied in fungicidally active amounts. The term “fungicidally effective amount” denotes an amount of the composition or of the mixtures, which is sufficient for controlling harmful fungi plants and which does not result in a substantial damage to the treated plants, young plants like seedlings, rooted/unrooted cuttings, plants derived from cell-culture or plant propagation materials, such as seeds. Such an amount can vary in a broad range and is dependent on various factors, such as the fungal species to be controlled, the treated plant species, the climatic conditions and the specific mixture used.

When the agrochemical compositions are used to be applied as foliar treatment or to the soil, preferably as foliar treatment. The application rates in foliar treatments are usually between 50 g/ha and 2000 g/ha, 100 g/ha and 2000 g/ha, 150 g/ha and 2000 g/ha, 600 g/ha and 2000 g/ha or 800 g/ha and 2000 g/ha or
between 50 g/ha and 1000 g/ha, 100 g/ha and 1000 g/ha, 150 g/ha and 1000 g/ha, 600 g/ha and 1000 g/ha or 800 g/ha and 1000 g/ha,
or between 50 g/ha and 800 g/ha, 100 g/ha and 800 g/ha, 150 g/ha and 800 g/ha, 600 g/ha and 800 g/ha or
between 150 g/ha and 1000 g/ha, 300 g/ha and 1000 g/ha, 600 g/ha and 1000 g/ha of fusaricidin comprising cells or spores in a volume
between 1000 L/ha and 100 L/ha, 600 L/ha and 100 L/ha, 400 L/ha and 100 L/ha, 200 L/ha and 100 L/ha or
between 1000 L/ha and 600 L/ha, 1000 L/ha and 400 L/ha or 1000 L/ha and 200 L/ha, or between 600 L/ha and 200 L/ha, 600 L/ha and 400 L/ha of a water based spraying liquid.

When the agrochemical compositions are employed in seed treatment, for example as seed coating, the application rates with respect to plant propagation material usually range from about 1×10¹ to 1×10¹² (or more) CFU/seed, preferably from about 1×10³ to about 1×10¹⁰ CFU/seed, and even more preferably from about 1×10³ to about 1×10⁶ CFU/seed. Alternatively, the application rates with respect to plant propagation material preferably range from about 1×10⁷ to 1×10¹⁶ (or more) CFU per 100 kg of seed, preferably from 1×10⁹ to about 1×10¹⁵ CFU per 100 kg of seed, even more preferably from 1×10¹¹ to about 1×10¹⁵ CFU per 100 kg of seed.

The Paenibacillus strains and the agrochemical compositions comprising these strains, respectively, are suitable as fungicides effective against a broad spectrum of phytopathogenic fungi, including soil-borne fungi, in particular from the classes of Plasmodiophoromycetes, Peronosporomycetes (syn. Oomycetes), Chytridiomycetes, Zygomycetes, Ascomycetes, Basidiomycetes, and Deuteromycetes (syn. Fungi imperfecti). They can be used in crop protection as foliar fungicides, fungicides for seed dressing, and soil fungicides.

The Paenibacillus strains and the agrochemical compositions comprising these strains are preferably useful in the control of phytopathogenic fungi on various cultivated plants, such as cereals, e.g. wheat, rye, barley, triticale, oats, or rice; beet, e.g. sugar beet or fodder beet; fruits, e. g. pomes (apples, pears, etc.), stone fruits (e.g. plums, peaches, almonds, cherries), or soft fruits, also called berries (strawberries, raspberries, blackberries, gooseberries, etc.); leguminous plants, e.g. lentils, peas, alfalfa, or soybeans; oil plants, e.g. oilseed rape, mustard, olives, sunflowers, coconut, cocoa beans, castor oil plants, oil palms, ground nuts, or soybeans; cucurbits, e.g. squashes, cucumber, or melons; fiber plants, e.g. cotton, flax, hemp, or jute; citrus fruits, e.g. oranges, lemons, grapefruits, or mandarins; vegetables, e.g. spinach, lettuce, asparagus, cabbages, carrots, onions, tomatoes, potatoes, cucurbits, or paprika; lauraceous plants, e.g. avocados, cinnamon, or camphor; energy and raw material plants, e.g. corn, soybean, oilseed rape, sugar cane, or oil palm; corn; tobacco; nuts; coffee; tea; bananas; vines (table grapes and grape juice grape vines); hop; turf; sweet leaf (also called Stevia); natural rubber plants; or ornamental and forestry plants, e.g. flowers, shrubs, broad-leaved trees, or evergreens (conifers, eucalypts, etc.); on the plant propagation material, such as seeds; and on the crop material of these plants.

More preferably, the Paenibacillus strains and the agrochemical compositions comprising these strains are used for controlling fungi on field crops, such as potatoes, sugar beets, tobacco, wheat, rye, barley, oats, rice, corn, cotton, soybeans, oilseed rape, legumes, sunflowers, coffee or sugar cane; fruits; vines; grapes for wine making or fruit grapes, ornamentals; or vegetables, such as cucumbers, tomatoes, pepper, beans or squashes.

The term “plant propagation material” is to be understood to denote all the generative parts of the plant, such as seeds; and vegetative plant materials, such as cuttings and tubers (e.g. potatoes), which can be used for the multiplication of the plant. This includes seeds, roots, fruits, tubers, bulbs, rhizomes, shoots, sprouts and other parts of plants; including seedlings and young plants to be transplanted after germination or after emergence from soil.

Accordingly, one embodiment of the invention is plant propagation material comprising the Paenibacillus strains or comprising a coating of an agrochemical composition comprising Paenibacillus strains. Preferably the plant propagation material are young plants, like seedlings, rooted/unrooted cuttings, plants derived from cell-culture. Even more preferred the plant propagation material is from fruit or vegetable plant species, including grapes.

According to the invention all of the above cultivated plants are understood to comprise all species, subspecies, variants and/or hybrids which belong to the respective cultivated plants. For example, corn is also known as Indian corn or maize (Zea mays) which comprises all kinds of corn such as field corn and sweet corn. According to the invention all maize or corn subspecies and/or varieties are comprised, in particular flour corn (Zea mays var. amylacea), popcorn (Zea mays var. everta), dent corn (Zea mays var. indentata), flint corn (Zea mays var. indurata), sweet corn (Zea mays var. saccharata and var. rugosa), waxy corn (Zea mays var. ceratina), amylomaize (high amylose Zea mays varieties), pod corn or wild maize (Zea mays var. tunicata) and striped maize (Zea mays var. japonica). These subspecies and/or varieties comprise also special growth forms like short stature corn The skilled person knows similar variants of other plant species, like, sweet pepper, determinate or indeterminate soybean, and others.

Accordingly, the invention provides for methods for controlling, suppressing or preventing fungal infection of plants comprising the steps of
a) providing a mixture comprising a1) a pure culture of a Paenibacillus species strain of the invention and a2) a pure culture of a microorganism which is sensitive to polymyxin or is sensitive to tridecaptin or is sensitive to polymyxin and tridecaptin or is sensitive to the presence of the parent strain of the Paenibacillus species strain of the invention and
b) applying an effective amount of a mixture of step a) to the plant pathogenic fungi, their habitat, the host plants, plant propagation material or the soil used to grow the plant.

Preferably the Paenibacillus species strain a1) and, if applicable the microorganism a2) are applied in the form of spores.

The Paenibacillus strains and the agrochemical compositions comprising these strains, respectively, are particularly suitable for controlling the following causal agents of plant diseases: Albugo spp. (white rust) on ornamentals, vegetables (e.g. A. candida) and sunflowers (e.g. A. tragopogonis); Alternaria spp. (Alternaria leaf spot) on vegetables (e.g. A. dauci or A. porri), oilseed rape (A. brassicicola or brassicae), sugar beets (A. tenuis), fruits (e.g. A. grandis), rice, soybeans, potatoes and tomatoes (e.g. A. solani, A. grandis or A. alternata), tomatoes (e.g. A. solani or A. alternata) and wheat (e.g. A. triticina); Aphanomyces spp. on sugar beets and vegetables; Ascochyta spp. on cereals and vegetables, e.g. A. tritici (anthracnose) on wheat and A. hordei on barley; Aureobasidium zeae (syn. Kapatiella zeae) on corn; Bipolaris and Drechslera spp. (teleomorph: Cochliobolus spp.), e.g. Southern leaf blight (D. maydis) or Northern leaf blight (B. zeicola) on corn, e.g. spot blotch (B. sorokiniana) on cereals and e.g. B. oryzae on rice and turfs; Blumeria (formerly Erysiphe) graminis (powdery mildew) on cereals (e.g. on wheat or barley); Botrytis cinerea (teleomorph: Botryotinia fuckeliana: grey mold) on fruits and berries (e.g. strawberries), vegetables (e.g. lettuce, carrots, celery and cabbages); B. squamosa or B. allii on onion family), oilseed rape, ornamentals (e.g. B eliptica), vines, forestry plants and wheat; Bremia lactucae (downy mildew) on lettuce; Ceratocystis (syn. Ophiostoma) spp. (rot or wilt) on broad-leaved trees and evergreens, e.g. C. ulmi (Dutch elm disease) on elms; Cercospora spp. (Cercospora leaf spots) on corn (e.g. Gray leaf spot: C. zeae-maydis), rice, sugar beets (e.g. C. beticola), sugar cane, vegetables, coffee, soybeans (e.g. C. sojina or C. kikuchii) and rice; Cladobotryum (syn. Dactylium) spp. (e.g. C. mycophilum (formerly Dactylium dendroides, teleomorph: Nectria albertinii, Nectria rosella syn. Hypomyces rosellus) on mushrooms; Cladosporium spp. on tomatoes (e.g. C. fulvum: leaf mold) and cereals, e.g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e.g. C. sativus, anamorph: B. sorokiniana) and rice (e.g. C. miyabeanus, anamorph: H. oryzae); Colletotrichum (teleomorph: Glomerella) spp. (anthracnose) on cotton (e.g. C. gossypii), corn (e.g. C. graminicola: Anthracnose stalk rot), soft fruits, potatoes (e.g. C. coccodes: black dot), beans (e.g. C. lindemuthianum), soybeans (e.g. C. truncatum or C. gloeosporioides), vegetables (e.g. C. lagenarium or C. capsici), fruits (e.g. C. acutatum), coffee (e.g. C. coffeanum or C. kahawae) and C. gloeosporioides on various crops; Corticium spp., e.g. C. sasakii (sheath blight) on rice; Corynespora cassiicola (leaf spots) on soybeans, cotton and ornamentals; Cycloconium spp., e.g. C. oleaginum on olive trees; Cylindrocarpon spp. (e.g. fruit tree canker or young vine decline, teleomorph: Nectria or Neonectria spp.) on fruit trees, vines (e.g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e.g. D. phaseolorum (damping off) on soybeans; Drechslera (syn. Helminthosporium, teleomorph: Pyrenophora) spp. on corn, cereals, such as barley (e.g. D. teres, net blotch) and wheat (e.g. D. tritici-repentis: tan spot), rice and turf; Esca (dieback, apoplexy) on vines, caused by Formitiporia (syn. Phellinus) punctata, F. mediterranea, Phaeomoniella chlamydospora (formerly Phaeoacremonium chlamydosporum), Phaeoacremonium aleophilum and/or Botryosphaeria obtusa; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e.g. E. pisi), such as cucurbits (e.g. E. cichoracearum), cabbages, oilseed rape (e.g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e.g. E. turcicum); Fusarium (teleomorph: Gibberella) spp. (wilt, root or stem rot) on various plants, such as F. graminearum or F. culmorum (root rot, scab or head blight) on cereals (e.g. wheat or barley), F. oxysporum on tomatoes, F. solani (f. sp. glycines now syn. F. virguliforme) and F. tucumaniae and F. brasiliense each causing sudden death syndrome on soybeans, and F. verticillioides on corn; Gaeumannomyces graminis (take-all) on cereals (e.g. wheat or barley) and corn; Gibberella spp. on cereals (e.g. G. zeae) and rice (e.g. G. fujikuroi: Bakanae disease); Glomerella cingulata on vines, pome fruits and other plants and G. gossypii on cotton; Grainstaining complex on rice; Guignardia bidwellii (black rot) on vines; Gymnosporangium spp. on rosaceous plants and junipers, e.g. G. sabinae (rust) on pears; Helminthosporium spp. (syn. Drechslera, teleomorph: Cochliobolus) on corn, cereals, potatoes and rice; Hemileia spp., e.g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e.g. wheat or barley); Microsphaera diffusa (powdery mildew) on soybeans; Monilinia spp., e.g. M. laxa, M. fructicola and M. fructigena (syn. Monilia spp.: bloom and twig blight, brown rot) on stone fruits and other rosaceous plants; Mycosphaerella spp. on cereals, bananas, soft fruits and ground nuts, such as e.g. M. graminicola (anamorph: Zymoseptoria tritici formerly Septoria tritici: Septoria blotch) on wheat or M. fijiensis (syn. Pseudocercospora fijiensis: black Sigatoka disease) and M. musicola on bananas, M. arachidicola (syn. M. arachidis or Cercospora arachidis), M. berkeleyi on peanuts, M. pisi on peas and M. brassiciola on brassicas; Peronospora spp. (downy mildew) on cabbage (e.g. P. brassicae), oilseed rape (e.g. P. parasitica), onions (e.g. P. destructor), tobacco (P. tabacina) and soybeans (e.g. P. manshurica); Phakopsora pachyrhizi and P. meibomiae (soybean rust) on soybeans; Phialophora spp. e.g. on vines (e.g. P. tracheiphila and P. tetraspora) and soybeans (e.g. P. gregata: stem rot); Phoma lingam (syn. Leptosphaeria biglobosa and L. maculans: root and stem rot) on oilseed rape and cabbage, P. betae (root rot, leaf spot and damping-off) on sugar beets and P. zeae-maydis (syn. Phyllostica zeae) on corn; Phomopsis spp. on sunflowers, vines (e.g. P. viticola: can and leaf spot) and soybeans (e.g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e.g. P. capsici), soybeans (e.g. P. megasperma, syn. P. sojae), potatoes and tomatoes (e.g. P. infestans: late blight) and broad-leaved trees (e.g. P. ramorum: sudden oak death); Plasmodiophora brassicae (club root) on cabbage, oilseed rape, radish and other plants; Plasmopara spp., e.g. P. viticola (grapevine downy mildew) on vines and P. halstedii on sunflowers; Podosphaera spp. (powdery mildew) on rosaceous plants, hop, pome and soft fruits (e.g. P. leucotricha on apples) and curcurbits (P. xanthii); Polymyxa spp., e.g. on cereals, such as barley and wheat (P. graminis) and sugar beets (P. betae) and thereby transmitted viral diseases; Pseudocercosporella herpotrichoides (syn. Oculimacula yallundae, O. acuformis: eyespot, teleomorph: Tapesia yallundae) on cereals, e.g. wheat or barley; Pseudoperonospora (downy mildew) on various plants, e.g. P. cubensis on cucurbits or P. humili on hop; Pseudopezicula tracheiphila (red fire disease or ‘rotbrenner’, anamorph: Phialophora) on vines; Puccinia spp. (rusts) on various plants, e.g. P. triticina (brown or leaf rust), P. striiformis (stripe or yellow rust), P. hordei (dwarf rust), P. graminis (stem or black rust) or P. recondita (brown or leaf rust) on cereals, such as e.g. wheat, barley or rye, P. kuehnii (orange rust) on sugar cane and P. asparagi on asparagus; Pyrenopeziza spp., e.g. P. brassicae on oilseed rape; Pyrenophora (anamorph: Drechslera) tritici-repentis (tan spot) on wheat or P. teres (net blotch) on barley; Pyricularia spp., e.g. P. oryzae (teleomorph: Magnaporthe grisea: rice blast) on rice and P. grisea on turf and cereals; Pythium spp. (damping-off) on turf, rice, corn, wheat, cotton, oilseed rape, sunflowers, soybeans, sugar beets, vegetables and various other plants (e.g. P. ultimum or P. aphanidermatum) and P. oligandrum on mushrooms; Ramularia spp., e.g. R. collo-cygni (Ramularia leaf spots, Physiological leaf spots) on barley, R. areola (teleomorph: Mycosphaerella areola) on cotton and R. beticola on sugar beets; Rhizoctonia spp. on cotton, rice, potatoes, turf, corn, oilseed rape, potatoes, sugar beets, vegetables and various other plants, e.g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis and R. commune (scald) on barley, rye and triticale; Sarocladium oryzae and S. attenuatum (sheath rot) on rice; Sclerotinia spp. (stem rot or white mold) on vegetables (S. minor and S. sclerotiorum) and field crops, such as oilseed rape, sunflowers (e.g. S. sclerotiorum) and soybeans, S. rolfsii (syn. Athelia rolfsii) on soybeans, peanut, vegetables, corn, cereals and ornamentals; Septoria spp. on various plants, e.g. S. glycines (brown spot) on soybeans, S. tritici (syn. Zymoseptoria tritici, Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setosphaeria spp. (leaf blight) on corn (e.g. S. turcicum, syn. Helminthosporium turcicum) and turf; Sphacelotheca spp. (smut) on corn, (e.g. S. reiliana, syn. Ustilago reiliana: head smut), sorghum und sugar cane; Sphaerotheca fuliginea (syn. Podosphaera xanthii: powdery mildew) on cucurbits; Spongospora subterranea (powdery scab) on potatoes and thereby transmitted viral diseases; Stagonospora spp. on cereals, e.g. S. nodorum (Stagonospora blotch, teleomorph: Leptosphaeria [syn. Phaeosphaeria] nodorum, syn. Septoria nodorum) on wheat; Synchytrium endobioticum on potatoes (potato wart disease); Taphrina spp., e.g. T. deformans (leaf curl disease) on peaches and T. pruni (plum pocket) on plums; Thielaviopsis spp. (black root rot) on tobacco, pome fruits, vegetables, soybeans and cotton, e.g. T. basicola (syn. Chalara elegans); Tilletia spp. (common bunt or stinking smut) on cereals, such as e.g. T. tritici (syn. T. caries, wheat bunt) and T. controversa (dwarf bunt) on wheat; Trichoderma harzianum on mushrooms; Typhula incarnata (grey snow mold) on barley or wheat; Urocystis spp., e.g. U. occulta (stem smut) on rye; Uromyces spp. (rust) on vegetables, such as beans (e.g. U. appendiculatus, syn. U. phaseoli), sugar beets (e.g. U. betae or U. beticola) and on pulses (e.g. U. vignae, U. pisi, U. viciae-fabae and U. fabae); Ustilago spp. (loose smut) on cereals (e.g. U. nuda and U. avaenae), corn (e.g. U. maydis: corn smut) and sugar cane; Venturia spp. (scab) on apples (e.g. V. inaequalis) and pears; and Verticillium spp. (wilt) on various plants, such as fruits and ornamentals, vines, soft fruits, vegetables and field crops, e.g. V. longisporum on oilseed rape, V. dahliae on strawberries, oilseed rape, potatoes and tomatoes, and V. fungicola on mushrooms; Zymoseptoria tritici on cereals.

The Paenibacillus strains and the agrochemical compositions comprising these strains are particularly suitable for controlling the following phytophathogenic fungi genera; Alternaria, Botrytis, Venturia, Leptosphaeria, Fusarium, Rhizoctonia, Phytophthora, Pythium, Colletotrichum, Pyricularia, Sclerotinia, Zymoseptoria.

The Paenibacillus strains and the agrochemical compositions comprising these strains are particularly suitable for controlling the following phytophathogenic fungi, Alternaria solanum, Alternaria alternata, Alternaria brassicae, Alternaria brassicicola, Alternaria citri, Alternaria mali, Botrytis cinerea, Botrytis allii, Botrytis fabae, Botrytis squamosa, Venturia inaequalis, Venturia effusa, Venturia carpophila, Venturia pyrina, Leptosphaeria maculans, Leptosphaeria nodorum Fusarium oxysporum, Fusarium graminearum, Fusarium verticillioides, Rhizoctonia solani, Phytophthora infestans, Phytophthora capsici, Phytophthora fragariae, Phytophthora nicotianae, Phytophthora sojae, Pythium ultimum, Pythium acanthicum, Pythium deliense, Pythium graminicola, Pythium heterothallicum, Pythium hypogynum, Pythium middletonii Colletotrichum orbiculare, Colletotrichum capsici, Colletotrichum coccodes, Colletotrichum fragariae, Colletotrichum lindemuthianum, Pyricularia oryzae, Sclerotinia sclerotiorum, Sclerotinia minor, Sclerotinia trifoliorum, Zymoseptoria tritici.

The Paenibacillus strains and the agrochemical compositions comprising these strains are particularly suitable for controlling the following phytophathogenic fungi, Alternaria solanum, Botrytis cinerea, Venturia inaequalis, Leptosphaeria maculans, Leptosphaeria nodorum, Fusarium oxysporum, Fusarium graminearum, Rhizoctonia solani, Phytophthora infestans, Pythium ultimum, Colletotrichum orbiculare, Pyricularia oryzae, Sclerotinia sclerotiorum and Zymoseptoria tritici.

The Paenibacillus strains and the agrochemical compositions comprising these strains are particularly suitable for controlling the following phytophathogenic fungi, Alternaria solanum, Botrytis cinerea, Leptosphaeria nodorum, Phytophthora infestans, Colletotrichum orbiculare, Pyricularia oryzae, Sclerotinia sclerotiorum and Zymoseptoria tritici.

The antifungal effect of the Paenibacillus strains and the agrochemical compositions comprising these strains can be enhanced by combining them with fungicides and other biologicals who have shown to have a positive effect with Paenibacillus strains and/or fusaricidins. Such fungicides and biologicals have been disclosed for example in WO2014085576, WO2017/137351, WO2017/137353, WO2020154813, WO2021097162 and WO2022/136003. Further preferred combinations are mixtures of the Paenibacillus strains and other microorganisms as disclosed herein.

Paenibacillus species strains are known for their capability to promote plant growth, e.g. via production of plant hormones, mobilization of plant nutrients or nitrogen fixation. This effect can be enhanced by combining these Paenibacillus species strains with other microorganisms, which may for example also fix nitrogen or by combining them with fertilizers, similar to the embodiments disclosed in WO2019/155253.

Accordingly, the invention comprises also a method to enhance plant growth comprising the steps of a) providing a pure culture of a Paenibacillus species strain of the invention or applying an effective amount of a mixture comprising a1) a pure culture of a Paenibacillus species strain of the invention and a2) a pure culture of a microorganism which is sensitive to polymyxin or is sensitive to tridecaptin or is sensitive to polymyxin and tridecaptin or is sensitive to the presence of the parent strain of the Paenibacillus species strain of the invention, and
b) applying an effective amount of a pure culture of the Paenibacillus species strain of the invention or applying an effective amount of a mixture comprising a1) a pure culture of a Paenibacillus species strain of the invention and a2) a pure culture of a microorganism which is sensitive to polymyxin or is sensitive to tridecaptin or is sensitive to polymyxin and tridecaptin or is sensitive to the presence of the parent strain of the Paenibacillus species strain of the invention, to the plant, plant propagation material or the soil used to grow the plant.

Another part of the invention is a kit of at least two parts comprising a) a pure culture of a Paenibacillus species strain of the invention in a first concentrated form and b) comprising a pure culture of a microorganism which is sensitive to polymyxin or is sensitive to tridecaptin or is sensitive to polymyxin and tridecaption or is sensitive the presence of the parent strain of the Paenibacillus species strain of the invention in a second concentrated form and comprising c) a set of instructions on how to use a) and b) to prepare a mixture to be used in a method for controlling, suppressing or preventing fungal infection of plants or to be used in a method to enhance plant growth.

A further part of the invention is plant propagation material comprising a pure culture of a Paenibacillus species strain of the invention or a mixture comprising a1) a pure culture of a microorganism which is sensitive to polymyxin or is sensitive to tridecaptin or is sensitive to polymyxin and tridecaption or is sensitive the presence of the parent strain of the Paenibacillus species strain of the invention.

A further part of the invention is plant propagation material comprising a whole culture broth, of a Paenibacillus species strain of the invention, a cell-free culture broth of the Paenibacillus species strain of the invention or a cell-free extract of the Paenibacillus species strain of the invention.

The Paenibacillus species strain of the invention produce less, or no DAB comprising compounds and are therefore especially suited to be used in processes, in which the production of DAB comprising compounds is not intended or even not desirable.

Such processes are for example the production of valuable product like enzymes used in animal feed, backing, detergents or other industries, like phytases, lipases, proteases, glucanases, xylanases or chitinases.

Another application of the Paenibacillus species strain of the invention is the production of bacterial spores for technical purposes, like spore display technologies.

Other valuable products of interest may be exopolysaccharides (EPS) produced by Paenibacillus species strains, like levan, curdlan or paenan. Further valuable products of interest are fusaricidin compounds, antifungal peptides, lanthionines or basic chemicals like 2,3-Butandiol, lactic acid or acetoin.

Accordingly, another part of the invention are methods for the production of valuable products comprising the steps of a) Paenibacillus species strain of the invention, b) culturing the Paenibacillus species strain of the invention under conditions which allow the production of the valuable product and c) harvesting the valuable product.

Harvesting the valuable product refers to a process in which the valuable product of interest is separated from the rest of the culture broth and the bacterial biomass produced during the culturing step. Depending on the nature of the product of interest the harvesting step can require further purification steps known in the art which depend on the degree of purification intended e.g., for some applications a simple step resulting in an enhanced concentration of the product of interest will suffice, while for other applications purification steps are added to produce a purified product basically devoid of other compounds.

The Paenibacillus species strains of the invention produce less or no DAB comprising compounds but regain this capacity if they are supplied with DAB. Thus, the Paenibacillus species strains of the invention can also be used in a method for the production of at least one DAB comprising compound, comprising the steps of a) providing a Paenibacillus species strains of the invention derived from a parent strain which has the capacity to produce the DAB comprising compound of interest, b) culturing the Paenibacillus species strains of the invention under conditions which allow the production of the DAB comprising compound of interest and providing the Paenibacillus species strains of the invention with sufficient amounts of DAB to allow the production of the DAB comprising compound of interest and c) harvesting the DAB comprising compound of interest.

Exopolysaccharides of bacterial origin have been used in many applications. Many of those applications make use of their capacity to alter rheological properties of liquids. This is for example useful in subterraneous oil and gas extraction, food, feed, cosmetics, and pharmaceutical preparations. In other applications EPS are applied as scaffolds or matrices for example in tissue engineering, drug delivery or in wound dressings.

Moreover, exopolysaccharides from Paenibacillus sp. were applied as antitumor agent, antioxidant or flocculant e.g., in wastewater treatment, see Hong et al., 2014, Antioxidant and antitumor activities of-glucan-rich exopolysaccharides with different molecular weight from Paenibacillus polymyxa JB115. J. Korean Soc. Appl. Biol. Chem. 57, 105-112, Liu et al., 2009, Production, characterization and antioxidant activities in vitro of exopolysaccharides from endophytic bacterium Paenibacillus polymyxa EJS-3. Carbohydr. Polym., 78, 275-281 and Tang et al., Production, purification and application of polysaccharide-based bioflocculant by Paenibacillus mucilaginosus. Carbohydr. Polym. 2014, 113, 463-470.

EPS of bacterial origin, like xanthan, succinoglycan or alginate, have also been used in many applications in agriculture e.g., in microencapsulation, in seed coatings, coating of roots, plants and seedlings to prevent desiccation, to improve the aggregation and water-holding capacity of soil, to improve the viability of microorganisms, to increases clinginess and permanence of pesticides or fertilizers, to modify the droplet size of spraying solutions, and to maintain homogeneous agrochemical suspensions. EPS of Paenibacillus are especially suited to be used for agricultural applications, because they have been shown to have useful effects on plants e.g., see Grinev et al., 2020, Isolation, structure, and potential biotechnological applications of the exopolysaccharide from Paenibacillus polymyxa 92, Carbohydrate Polymers, 232, 115780 and Yegorenkova et al. 2021, Effect of exopolysaccharides of Paenibacillus polymyxa rhizobacteria on physiological and morphological variables of wheat seedlings, Journal of Microbiology, 59, 8, 729-735, They also have been shown to induce plant acquired resistance and have antagonizing effects on fungal plant pathogens e.g., see Zhao et al., 2020, Tobacco-acquired resistance induced by an exopolysaccharide of Paenibacillus kribbensis PS04 against bacterial wilt, BIOCONTROL SCIENCE AND TECHNOLOGY, 30, 4, 370-383, and Timmusk et al., 2019, Paenibacillus polymyxa biofilm polysaccharides antagonise Fusarium graminearum. Sci Rep. 2019 Jan. 24; 9(1):662,

For many of the above-described applications it would be of advantage to use EPS of an Paenibacillus species, which is not burdened with the presence of an unnecessary amount of antimicrobial substances like colistin, tridecaptin M or both and is preferably not burdened with an unnecessary amount of a polymyxin or a tridecaptin or even more preferred any DAB comprising compound.

Accordingly, another part of the invention are EPS of a Paenibacillus species which do not comprise colistin, tridecaptin M or both and preferably do not comprise a polymyxin or tridecaptin and even more preferred do not comprise any DAB comprising compound.

The phrase “do not comprise” does not mean that these EPS are absolutely free of the respective DAB comprising compound but means that the content of the DAB comprising compound is in a mass fraction of less than 0.1 mg/kg of EPS, preferably less than 0.01 mg/kg EPS, more preferred less than 1 μg/kg EPS and even more preferred less than 0.1 μg/kg EPS.

Such exopolysaccharides comprise at least one of curdlan, levan or paenan, preferably they comprise levan or paenan or levan and paenan. and may also comprise at least one of a) remaining DNA of the Paenibacillus species strain used for its production or b) remaining protein of the Paenibacillus species strain used for its production, c) at least one fusaricidin, d) at least one lanthipeptide produced by a Paenibacillus species strain or e) any combination of at least two of a) to d).

Another part of the invention are compositions for subterraneous oil and gas extraction, compositions for treatment of soils for gardening or agriculture, food, feed, cosmetics, pharmaceutical preparations, scaffolds or matrices for use in tissue engineering or drug delivery or in wound dressings, fertilizers, spraying solutions, agrochemical compositions, in particular agrochemical suspension concentrates and seed coatings, comprising Paenibacillus produced EPS not comprising colistin, tridecaptin M or both and preferably not comprising a polymyxin or tridecaptin and even more preferred not comprising any DAB comprising compound.

A further part of the invention is the use of Paenibacillus produced EPS not comprising colistin, tridecaptin M or both and preferably not comprising a polymyxin or a tridecaptin and even more preferred not comprising any DAB comprising compound in compositions for the use in subterraneous oil and gas extractions, for treatment of soils in gardening or agriculture, in food, feed, cosmetics, pharmaceutical preparations, scaffolds or matrices for use in tissue engineering or drug delivery or in wound dressings, fertilizers, spraying solutions, agrochemical compositions, in particular agrochemical suspension concentrates and seed coatings

Another part of the invention are compositions comprising Paenibacillus produced EPS not comprising colistin, tridecaptin M or both and preferably not comprising a polymyxin or tridecaptin and even more preferred not comprising any DAB comprising compound in compositions for the use in subterraneous oil and gas extractions, for treatment of soils in gardening or agriculture, in food, feed, cosmetics, pharmaceutical preparations, scaffolds or matrices for use in tissue engineering or drug delivery or in wound dressings, fertilizers, spraying solutions, agrochemical compositions, in particular agrochemical suspension concentrates and seed coatings, In one embodiment of the invention, the Paenibacillus produced EPS not comprising colistin, tridecaptin M or both and preferably not comprising a polymyxin or a tridecaptin and even more preferred not comprising any DAB comprising compound are used in connection with a microorganism which is sensitive to the presence of colistin, tridecaptin M or both, preferably is sensitive to the presence of at least one polymyxin or at least one tridecaptin and even more preferred is sensitive to the presence of at least one DAB comprising compound. Such microorganisms preferably have a use in agriculture, such as microorganisms having an insecticidal, antifungal, antioomycotal effect or are used to improve the availably of nutrients to a plant, such as microorganisms mobilizing phosphorous or fixing nitrogen.

Preferred applications of Paenibacillus produced EPS in combination with further microorganisms are the microencapsulation of these microorganism or for enhancing the survival rate of such microorganisms during storage or on seeds or to enhance endosymbiosis of nitrogen fixing bacteria. Examples of such applications using different exopolysaccharides can be found in WO201957958 or in Rodrigues et al, 2015, Rhizobium tropici exopolysaccharides as carriers improve the symbiosis of cowpea-Bradyrhizobium-Paenibacillus, African Journal of Microbiology Research, Vol. 9(37), pp. 2037-2050, Article Number: 2871DA655586.

Thus, the invention comprises compositions comprising:
a) Paenibacillus produced EPS not comprising colistin, tridecaptin M or both and preferably not comprising a polymyxin or tridecaptin and even more preferred not comprising any DAB comprising compound and
b) a pure culture of a microorganism which is not a Paenibacillus species strain.

Such microorganism is preferably a microorganism which is sensitive to the presence of colistin, tridecaptin M or both and preferably is sensitive to the presence at least one polymyxin or at least one tridecaptin and even more preferred is sensitive to the presence of at least one DAB comprising compound.

In one embodiment such microorganisms belong to the genus Azotobacter, Azospirillum, Pseudomonas, Burkholderia, Paraburkholderia, Rhizobium, Bradyrhizobium, Sinorhizobium, Mesorhizobium, Bacillus or Streptomyces, preferably they belong to the species and strain described above as potential mixture partners for Paenibacillus strains of the invention.

The invention also provides a method of exopolysaccharide production, comprising a) growing a microorganism according to the invention, b) optionally separating the microorganism from the exopolysaccharide and c) harvesting the produced exopolysaccharide. Suitable methods of growing a microorganism of the present invention, that is, fermentation methods, are generally known to the person skilled in the art. It is a particular advantage that the improved yield in exopolysaccharides can be achieved according to the invention without fundamental changes in corresponding fermentation processes. Methods to harvest the exopolysaccharides are also well known in the art, see e.g., Donot et al., 2012, Microbial exopolysaccharides: Main examples of synthesis, excretion, genetics and extraction, Carbohydrate Polymers, 87, 2, 951-962 or in CN113480672.

Separation of the EPS from the cells can be performed via mechanical means like centrifugation.

The EPS is usually precipitated from the supernatant with a surplus of an organic solvent. In one embodiment the organic solvent is ethanol or acetone having a volume of three or more times the volume of the supernatant.

The precipitated EPS can again be separated via mechanical means.

Optionally, the EPS can be purified by means available in the art e.g., with further washing steps, gel filtration or anion-exchange chromatography. The degree of purity of the EPS is usually adapted to the intended purpose. Applications in pharmaceutical or food and feed applications generally require a higher purity of the EPS than applications in agriculture compositions or in combinations with other microorganisms.

The separation of spores and cells may be supported by the addition of amphiphilic sulfonate and/or an amphiphilic sulfate to support the formation of aggregates, which are more readily separated from the EPS comprising supernatant, e.g. as disclosed in WO2017/151742.

Accordingly, the invention comprises a method of purifying an exopolysaccharide comprising: a) providing a culture of a Paenibacillus species strain producing an EPS not comprising colistin, tridecaptin M or both and preferably not comprising a polymyxin or a tridecaptin and even more preferred not comprising any DAB comprising compound,
b) optionally mixing an amphiphilic sulfonate and/or an amphiphilic sulfate with the cell culture to induce the formation of aggregates,
c) separating the Paenibacillus species strain cells and spores from the culture to generate a supernatant fraction and a pellet fraction,
d) separating the supernatant fraction from the pellet fraction,
e) adding an organic solvent, preferably an alcohol or acetone, to the supernatant fraction to precipitate the EPS, and
f) removing the precipitated EPS from the supernatant fraction.

The precipitated EPS can then be further purified by methods known in the art to reach the intended degree of purity for the purpose.

The invention comprises also a method to create a Paenibacillus species strain of the invention. Such a strain can be
a) a Paenibacillus species strain comprising only one genetic locus comprising at least, in the order of rising preference, 80%, 81%, 82%, 83%, 84%, 85%, 86%, 87%, 88%, 89%, 90%, 91%, 92%, 93%, 94%, 95%, 96%, 97%, 98%, or 99% sequence identity to a polynucleotide sequence of any one of SEQ IN NOs: 222, 223, 224, 225, 413, 414, 415, 416, 417, 418 and not comprising an open reading frame for a diaminobutyrate-2-oxoglutarate-transaminase comprising an amino acid sequence of at least, in the order of rising preference, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 217, or
b) a Paenibacillus species strain which does not comprise an open reading frame for a diaminobutyrate-2-oxoglutarate-transaminase comprising an amino acid sequence of at least at least, in the order of rising preference, 90%, 91%, 92%, 93%, 94%, 95%, 96%, 97%, 98%, or 99% sequence identity sequence identity to at least one of SEQ ID NO: 217, 218, 219, 220 or 221, or
c) a Paenibacillus species strain producing more of at least one glutamine comprising fusaricidin, paeniprolixin or paeniserine than of its respective asparagine comprising counterpart, or
d) a Paenibacillus species strain producing an EPS not comprising colistin, tridecaptin M or both and preferably not comprising a polymyxin or a tridecaptin and even more preferred not comprising any DAB comprising compound.

The method comprising the following steps of
a) providing a Paenibacillus species parent strain comprising an open reading frame for a diaminobutyrate-2-oxoglutarate-transaminase comprising an amino acid sequence of at least at least, in the order of rising preference, 90%, 91%, 92%, 93%, 94%, 95%, 96%, 97%, 98%, or 99% sequence identity sequence identity to at least one of SEQ ID NO: 217, 218, 219, 220 or 221, preferably this strain comprises at least two genomic loci comprising at least, in the order of rising preference, 80%, 81%, 82%, 83%, 84%, 85%, 86%, 87%, 88%, 89%, 90%, 91%, 92%, 93%, 94%, 95%, 96%, 97%, 98%, or 99% sequence identity to a polynucleotide sequence of any one of SEQ IN NOs: 222, 223, 224, 225, 413, 414, 415, 416, 417, 418,
b) providing a stress to a culture of the Paenibacillus species parent strain,
c) culturing the Paenibacillus species parent strain of b),
d) isolating a pure culture of a Paenibacillus species strain not producing colistin, tridecaptin M or both and preferably not comprising a polymyxin or a tridecaptin and even more preferred not comprising any DAB comprising compound.

The stress applied to the Paenibacillus species parent strain of step b) can any stress in the art that enhances the frequency of recombination in bacterial cells, in one embodiment the stress is a heat shock at 60° C. to 90° C. for about 5 to 40 minutes. In a different embodiment the stress is induced by exposing the Paenibacillus species parent strain to material from a fungus or oomycete. This material can be supernatant of a fungus or oomycete culture, material of dead fungus or oomycete cells or living fungal or oomycete cells.

In case living fungal or oomycote cells are used in step b) it is advisable to include an effective amount of a substance in the culture medium of step c) to prevent growth of the fungal or oomycote cells. The material is preferably from a filamentous fungus and preferably from a Botrytis species or from an oomycote such as a Phytophthora species. The substance to prevent the growth of the living fungal or oomycote cells is preferably a fungicide having activity against the fungal or oomycote species used in step b).

The culturing in step c) can be performed on solid or in liquid medium. Preferably step c) comprises several recultivation steps in which single colonies or diluted medium is used for recultivation. Each culture step preferably comprises at least two, preferably three days. Preferably step c) comprises at least three, preferably at least four recultivation steps. Preferably each recultivation step is designed to put additional stress on the growing cells due to limited supply of nutrients.

Several methods known in the art can be used to isolate and test pure cultures in step d) from cells grown at the, preferably last, recultivation step of step c). One simple method is to plate single colonies in an overlay assay using a bacterial lawn of an indicator strain. The indicator strain is selected from a microorganism being sensitive to the presence of colistin, tridecaptin M or both and preferably sensitive to the presence of at least one polymyxin or at least one tridecaptin and even more preferred sensitive to the presence of at least one DAB comprising compound. Preferably the indicator strain is selected to be sensitive to the presence of at least one DAB comprising compound produced by the Paenibacillus species parent strain of step a). The Paenibacillus species strain of interest in step d) can be isolated by picking a colony of a the Paenibacillus species which does not prevent the growth of the indicator strain used. Preferred indicator strains are E. coli strains or strains of microorganism mentioned herein as mixture partners for a Paenibacillus species strain of the invention.

While this disclosure contains many specific implementation details, these should not be construed as limitations on the scope of the subject matter or on the scope of what can be claimed, but rather as descriptions of features that can be specific to particular implementations. Certain features that are described in this disclosure in the context of separate implementations can also be implemented, in combination, in a single implementation. Conversely, various features that are described in the context of a single implementation can also be implemented in multiple implementations, separately, or in any suitable sub-combination. Moreover, although previously described features can be described as acting in certain combinations and even initially claimed as such, one or more features from a claimed combination can, in some cases, be excised from the combination, and the claimed combination can be directed to a sub-combination or variation of a sub-combination.

Particular embodiments of the subject matter have been described. Other implementations, alterations, and permutations of the described implementations are within the scope of the following claims as will be apparent to those skilled in the art.

Accordingly, the previously described example implementations do not define or constrain this disclosure. Other changes, substitutions, and alterations are also possible without departing from the spirit and scope of this disclosure.

Exemplary embodiments are:
1. A Paenibacillus species strain comprising only one genetic locus comprising at least 80% sequence identity to a polynucleotide sequence of any one of SEQ IN NOs: 222, 223, 224, 225, 413, 414, 415, 416, 417, 418 and not comprising an open reading frame for a diaminobutyrate-2-oxoglutarate-transaminase comprising an amino acid sequence of at least 80% sequence identity to SEQ ID NO: 217.
2. A Paenibacillus species strain as described in embodiment 1, not comprising a polynucleotide sequence encoding an amino acid sequence comprising at least 80% sequence identity to SEQ ID NO:1.
3. A Paenibacillus species strain as described in embodiments 1 or 2, which does not comprise a continuous genome fragment of, in the order of rising preference, less than 350, 340, 330, 320, 310 or 300 kilobases, which comprises a plurality of open reading frames encoding polypeptide sequences of at least 95% sequence identity
   e) to the amino acid sequences of SEQ ID NOs: 2, 3, 5, 7, 8, 9, 10, 11, 12, 13, 14, 15, 16, 17, 18, 19, 20, 21, 22, 23, 25, 27, 28, 30, 31, 32, 34, 36, 37, 38, 39, 40, 41, 44, 45, 47, 48, 50, 51, 52, 53, 54, 55, 58, 60, 61, 62, 63, 64, 65, 66, 67, 68, 69, 70, 72, 77, 78, 79, 80, 81, 82, 83, 84, 86, 87, 89, 91, 92, 93, 94, 95, 96, 97, 98, 100, 101, 102, 103, 104, 105, 107, 108, 109, 110, 111, 112, 113, 114, 115, 116, 117, 118, 119, 120, 122, 124, 125, 126, 127, 128, 129, 130, 132, 133, 134, 135, 136, 137, 138, 139, 140, 141, 142, 143, 144, 145, 146, 147, 148, 149, 150, 151, 152, 153, 154, 155, 156, 157, 158, 159, 161, 162, 163, 164, 165, 166, 167, 169, 170, 174, 175, 176, 177, 178, 180, 181, or
   f) to the amino acid sequences of SEQ ID NOs: 241, 242, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 309, 310, 311, 312, 313, 314, 315, 316, 317, 318, 319, 320, 321, 322, 323, 324, 325, 326, 327, 328, 329, 330, 331, 332, 333, 347, 348, 349, 350, 351, 352, 353, 354, 355, 356, 357, 358, 360, 363, 363, 364, 365, 366, 367, 369, 370, 371, 372, 373, 374, 375, 376, 377, 378, 379, 380, 381, 382, 383, 384, 385, 387, 388, 389, 390, 391, 392, 393, 394, 395, 396, 397, 398, 399, 400, 401, 402, 403, 404, 405, 406, 407.
4. A Paenibacillus species strain as described in any one of embodiments 1 to 3, being an Paenibacillus polymyxa strain and having an ANI-Value of at least 99.9% to the Paenibacillus polymyxa strain LU17007 deposited as DSM 26970 or to the Paenibacillus polymyxa strain SB3-1.
5. A Paenibacillus species strain as described in any one of embodiments 1 to 4 being derived from a parent strain, wherein the parent strain comprises an open reading frame for a diaminobutyrate-2-oxoglutarate_transaminase comprising an amino acid sequence of at least 80% sequence identity to SEQ ID NO: 217 and at least two loci each one comprising at least 80% sequence identity to at least one of the nucleotide sequences of SEQ ID NO: 222, 223, 224, 225, 413, 414, 415, 416, 417 and 418.
6. A Paenibacillus species strain as described in any one of embodiments 1 to 5 being derived from a parent strain, wherein the parent strain comprises an open reading frame encoding an amino acid sequence of at least 80% sequence identity to SEQ ID NO: 217 and an open reading frame encoding an amino acid sequence of at least 80% sequence identity to SEQ ID NO: 409, preferably encoded by a polynucleotide sequence of at least 80% sequence identity to SEQ ID NO: 411, and an open reading frame encoding an amino acid sequence of at least 80% sequence identity to SEQ ID NO: 410 preferably encoded by a polynucleotide sequence of at least 80% sequence identity to SEQ ID NO: 412.
7. A Paenibacillus species strain not comprising a polynucleotide sequence encoding an amino acid sequence of at least 80% sequence identity to SEQ ID NO: 1 and not comprising an open reading frame for a diaminobutyrate-2-oxoglutarate-transaminase comprising an amino acid sequence of at least 80% identity to SEQ ID NO: 217, derived from a parent strain, wherein the parent strain comprises an open reading frame for a diaminobutyrate-2-oxoglutarate-transaminase comprising an amino acid sequence of at least 80% sequence identity to SEQ ID NO: 217 and at least two loci each one comprising at least 80% sequence identity to at least one of the nucleotide sequences of SEQ ID NO: 222, 223, 224, 225, 413, 414, 415, 416, 417 and 418.
8. A Paenibacillus species strain as described in any one of embodiments 5 to 7, wherein the parent strain comprises a continuous genome fragment of, in the order of rising preference, less than 350, 340, 330, 320, 310 or 300 kilobases, which comprises a plurality of open reading frames encoding polypeptide sequences of at least 95% sequence identity
   g) each one of the amino acid sequences of SEQ ID NOs: 2, 3, 5, 7, 8, 9, 10, 11, 12, 13, 14, 15, 16, 17, 18, 19, 20, 21, 22, 23, 25, 27, 28, 30, 31, 32, 34, 36, 37, 38, 39, 40, 41, 44, 45, 47, 48, 50, 51, 52, 53, 54, 55, 58, 60, 61, 62, 63, 64, 65, 66, 67, 68, 69, 70, 72, 77, 78, 79, 80, 81, 82, 83, 84, 86, 87, 89, 91, 92, 93, 94, 95, 96, 97, 98, 100, 101, 102, 103, 104, 105, 107, 108, 109, 110, 111, 112, 113, 114, 115, 116, 117, 118, 119, 120, 122, 124, 125, 126, 127, 128, 129, 130, 132, 133, 134, 135, 136, 137, 138, 139, 140, 141, 142, 143, 144, 145, 146, 147, 148, 149, 150, 151, 152, 153, 154, 155, 156, 157, 158, 159, 161, 162, 163, 164, 165, 166, 167, 169, 170, 174, 175, 176, 177, 178, 180, 181, or
   h) each one of the amino acid sequences of SEQ ID NOs: 241, 242, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 309, 310, 311, 312, 313, 314, 315, 316, 317, 318, 319, 320, 321, 322, 323, 324, 325, 326, 327, 328, 329, 330, 331, 332, 333, 347, 348, 349, 350, 351, 352, 353, 354, 355, 356, 357, 358, 360, 363, 363, 364, 365, 366, 367, 369, 370, 371, 372, 373, 374, 375, 376, 377, 378, 379, 380, 381, 382, 383, 384, 385, 387, 388, 389, 390, 391, 392, 393, 394, 395, 396, 397, 398, 399, 400, 401, 402, 403, 404, 405, 406, 407.
9. A Paenibacillus species strain, preferably a Paenibacillus polymyxa strain, as described in any one of embodiments 5 to 8, wherein the parent strain has an ANI-Value of at least 99% to the Paenibacillus polymyxa strain LU17007 deposited as DSM 26970 or to the Paenibacillus polymyxa strain SB3-1.
10. A Paenibacillus species strain as described in embodiment 1 or 7, being any one of the Paenibacillus polymyxa strains of LU20754 deposited as DSM 33970, LU21132 deposited as DSM 33971, LU21514 deposited as DSM 33972, LU21552 deposited as DSM 33973, LU22377 deposited as DSM 33974, LU22378 deposited as DSM 33975, LU22379 deposited as DSM 33976, and LU22381 deposited as DSM 33977.
11. A mixture comprising a) a pure culture of a Paenibacillus species strain which does not produce a polymyxin and b) a pure culture of a microorganism which is sensitive to polymyxin.
12. A mixture as described in embodiment 11, wherein the Paenibacillus species strain of a) does not produce a polymyxin or a tridecaptin, or does not produce a polymyxin and does not produce a tridecaptin, and the second microorganism of b) is sensitive to a polymyxin or a tridecaptin or is sensitive to a polymyxin and a tridecaptin.
13. A mixture as described in embodiment 11 or 12, wherein the microorganism of b) is not sensitive to the presence of the Paenibacillus species strain of a) but is sensitive to the presence of the parent strain of the Paenibacillus species strain of a).
14. A mixture comprising a) a pure culture of a Paenibacillus species strain which does not comprise an open reading frame for a diaminobutyrate-2-oxoglutarate-transaminase comprising an amino acid sequence of at least 90% sequence identity to at least one of SEQ ID NO: 217, 218, 219, 220 or 221 and b) a pure culture of a microorganism which is sensitive to at least one of polymyxin, tridecaptin, polypeptin, paenilipoheptin, octapeptin, jolipeptin, gavaserin or saltavalin, preferably is sensitive to at least one of polymyxin, tridecaptin or both.
15. A mixture as described in any one of embodiments 11 to 14, wherein the Paenibacillus species strain of a) comprises no or only one locus encoding an amino acid sequence comprising at least 90% sequence identity to SEQ ID NO: 1 and does not produce a polymyxin or a tridecaptin or does not produce a polymyxin or a tridecaptin.
16. A mixture as described in any one of embodiments 11 to 15, wherein the parent strain of the Paenibacillus species strain of a) comprises at least one locus encoding an amino acid sequence comprising at least 90% sequence identity to SEQ ID NO: 1 and does produce at least one tridecaptin and does preferably produce at least one polymyxin and at least one tridecaptin.
17. A mixture as described in any one of embodiments 11 to 16 comprising a pure culture of a of a microorganism selected from Azotobacter, Azospirillum, Pseudomonas, Paraburkholderia, Rhizobium, Bradyrhizobium, Bacillus or Streptomyces.
18. A mixture as described in any one of embodiments 11 to 17 comprising a pure culture of a microorganism selected from: Pseudomonas koreensis, Pseudomonas gessardii, Pseudomonas chlororaphis, Paraburkholderia phytofirmans, Rhizobium phaseoli, Rhizobium radiobacter, Bradyrhizobium japonicum, Bacillus licheniformis, Bacillus subtilis, Bacillus amyloliquefaciens, Bacillus licheniformis, Bacillus mycoides, Bacillus firmus, Bacillus thuringiensis, Bacillus pumilus, Bacillus simplex, Streptomyces griseoviridis and Streptomyces lydicus.
19. A mixture as described in any one of embodiments 11 to 18 comprising a pure culture of a microorganism selected from: Pseudomonas koreensis, Paraburkholderia phytofirmans, Rhizobium phaseoli, Rhizobium radiobacter, Bradyrhizobium japonicum, Bacillus licheniformis, Bacillus subtilis, Bacillus velezensis and Streptomyces lydicus.
20. A Paenibacillus species strain producing more of at least one glutamine comprising fusaricidin, paeniprolixin or paeniserine than of its respective asparagine comprising counterpart.
21. A Paenibacillus species strain as described in embodiment 20, producing at least one of a) to e), wherein a) is producing more fusaricidin LiF04b (Fus B) than fusaricidin LiF04a (Fus A), b) producing more fusaricidin LiF03b than fusaricidin LiF03a, c) producing more fusaricidin LiF05b than fusaricidin LiF05a, d) producing more fusaricidin LiF06b than fusaricidin LiF06a, e) fusaricidin LiF07b than fusaricidin LiF07a.
22. A Paenibacillus species strain as described in embodiment 20 or 21, producing more fusaricidin LiF04b (Fus B) than fusaricidin LiF04a (Fus A).
23. A Paenibacillus species strain as described in any one of embodiments 20 to 22, comprising at least one of the characteristics described for the Paenibacillus polymyxa strain of claims 1 to 9.
24. An agrochemical composition comprising a Paenibacillus species strain as described in any one of embodiments 1 to 10 and/or any one of embodiments 20 to 23 or a mixture as described in any one of embodiments 11 to 19 and an auxiliary.
25. A whole culture broth, cell-free culture broth, spores or a cell-free extract of a Paenibacillus species strain as described in any one of embodiments 1 to 10 and/or any one of embodiments 20 to 23.
26. A method for controlling, suppressing or preventing fungal infection of plants comprising the steps of a) providing a pure culture of a Paenibacillus species strain as described in any one of embodiments 1 to 10 and/or any one of embodiments 20 to 23, or a mixture as described in any one of embodiments 11 to 19, an agrochemical composition as described in embodiment 24 or a whole culture broth, a cell-free culture broth or a cell-free extract as described in embodiment 25 and b) applying an effective amount of the providing a pure culture of a Paenibacillus species strain as described in any one of embodiments 1 to 10 and/or any one of embodiments 20 to 23, or a mixture as described in any one of embodiments 11 to 19, an agrochemical composition as described in embodiment 24 or a whole culture broth, a cell-free culture broth or a cell-free extract as described in embodiment 25 to the plant pathogenic fungi, their habitat, the host plants, plant propagation material or the soil used to grow the plant.
27. A method to enhance plant growth comprising the steps of a) providing a pure culture of a Paenibacillus species strain as described in any one of embodiments 1 to 10 and/or any one of embodiments 20 to 23, or a mixture as described in any one of embodiments 11 to 19 or an agrochemical composition as described in embodiment 24 or a whole culture broth, a cell-free culture broth or a cell-free extract as described in embodiment 25 and b) applying an effective amount of the pure culture of a Paenibacillus species strain as described in any one of embodiments 1 to 10 and/or any one of embodiments 20 to 23, or a mixture as described in any one of embodiments 11 to 19 or an agrochemical composition as described in embodiment 24 or a whole culture broth, a cell-free culture broth or a cell-free extract as described in embodiment 25 to the plant, plant propagation material or the soil used to grow the plant.
28. A kit of at least two parts comprising a) a pure culture of a Paenibacillus species strain as described in any one of embodiments 1 to 10 and/or any one of embodiments 20 to 23 in a first concentrated form and b) comprising a pure culture of a microorganism as described in any one of embodiments 11 to 19 in a second concentrated form and comprising c) a set of instructions on how to use a) and b) to prepare a mixture as described in any one of embodiments 11 to 19 to be used in a method as described in any one of embodiments 26 or 27.
29. An isolated Paenibacillus exopolysaccharide which does not comprise colistin, tridecaptin M or both and preferably do not comprise a polymyxin or tridecaptin and even more preferred do not comprise any DAB comprising compound.
30. An isolated Paenibacillus exopolysaccharide as described in embodiment 29, produced by a Paenibacillus strain as described in any one of embodiments 1 to 10 and/or any one of embodiments 20 to 23.
31. A composition comprising an isolated Paenibacillus exopolysaccharide as described in embodiment 29 or 30 and comprising at least one of a) an auxiliary, b) a pure culture of microorganism which is sensitive to the presence of colistin, tridecaptin M or both, preferably is sensitive to the presence of at least one polymyxin or at least one tridecaptin and even more preferred is sensitive to the presence of at least one DAB comprising compound, or c) a fertilizer.
32. A plant propagation material comprising a pure culture of a Paenibacillus species as described in any one of embodiments 1 to 10 and/or any one of embodiments 20 to 23, or a mixture as described in any one of embodiments 11 to 19 or an agrochemical composition as described in embodiment 24 or a whole culture broth, a cell-free culture broth or a cell-free extract as described in embodiment 25, or isolated Paenibacillus exopolysaccharide as described in embodiment 29 or 30, or a composition as described in embodiment 31.
33. A method of production of a valuable product comprising, a) culturing a Paenibacillus species strain as described in any one of embodiments 1 to 10 and/or any one of embodiments 20 to 23, and b) harvesting the valuable product.
34. A method as described in embodiment 33, wherein the valuable product is a) an enzyme or a protein, b) an antifungal compound, c) an exopolysaccharide, d) one or more DAB comprising compounds or e) 2,3-Butanediol, lactic acid or acetoin.
35. Use of a Paenibacillus species strain as described in any one of embodiments 1 to 10 and/or any one of embodiments 20 to 23 in a mixture as described in any one of embodiments 11 to 19, in a kit of parts as described in embodiment 28, on a plant propagation material or in a method as described in any one of embodiments 26, 27, 33 or 34.
36. Use of mixture as described in any one of embodiments 11 to 19 in a method as described in embodiment 26 or 27.
37. Use of an isolated Paenibacillus exopolysaccharide as described in embodiment 29 or 30 or a composition as described in embodiment 31 in a method as described in embodiment 26 or 27.
38. Use of an isolated Paenibacillus exopolysaccharide as described in embodiment 29 or 30 to prepare a cosmetic composition, a food or feed additive, a pharmaceutical or cosmetic composition, a pharmaceutical or cosmetic carrier, skin hydration composition, a flocculant, an antitumor agent, or as an antioxidant.
39. A method to create a Paenibacillus species strain as described in any one of embodiments 1 to 10 and/or any one of embodiments 20 to 23, comprising the following steps of a) providing a Paenibacillus species parent strain as described in any one of embodiments 5 to 9, b) providing stress to a culture of the Paenibacillus species parent strain of a), c) culturing the stressed Paenibacillus species parent strain of b) and d) isolating a pure culture of a Paenibacillus species strain not producing colistin, tridecaptin M or both and preferably not comprising a polymyxin or a tridecaptin and even more preferred not comprising any DAB comprising compound.

### DEPOSIT INFORMATION

Samples of the Paenibacillus sp. strains of the invention have been deposited by BASF SE (Germany) under the Budapest Treaty with the Deutsche Sammlung von Mikroorganismen und Zellkulturen (DSMZ):
1) Paenibacillus strain Lu17007 deposited under Acc. No. DSM 26970 on Feb. 20, 2013
2) Paenibacillus strain Lu20754 deposited under Acc. No. DSM 33970 on Jul. 16, 2021
3) Paenibacillus strain Lu21132 deposited under Acc. No. DSM 33971 on Jul. 16, 2021
4) Paenibacillus strain Lu21514 deposited under Acc. No. DSM 33972 on Jul. 16, 2021
5) Paenibacillus strain LU21552 deposited under Acc. No. DSM 33973 on Jul. 16 2021
6) Paenibacillus strain LU22377 deposited under Acc. No. DSM 33974 on Jul. 16 2021
7) Paenibacillus strain LU22378 deposited under Acc. No. DSM 33975 on Jul. 16, 2021
8) Paenibacillus strain LU22379 deposited under Acc. No. DSM 33976 on Jul. 16 2021
9) Paenibacillus strain LU22381 deposited under Acc. No. DSM 33977 on Jul. 16, 2021 Strains Lu20754, Lu21132, Lu21514, LU21552, LU22377, LU22378, LU22379 and LU22381 have been developed using strain Lu17007 as direct or indirect parent strain.

The Paenibacillus sp. strains have been deposited under conditions that assure that access to the culture will be available during the pendency of this patent application to one determined by the Commissioner of Patents and Trademarks to be entitled thereto under 37 C.F.R. § 1.14 and 35 U.S.C. § 122. However, it should be understood that the availability of a deposit does not constitute a license to practice the subject invention in derogation of patent rights granted by governmental action. The deposits have been made merely as a convenience for those of skill in the art and are not an admission that a deposit is required under 35 U.S.C. § 112.

### EXAMPLES

### Strains Used in the Examples

TABLE 4
Paenibacillus strains

| | Strain | | | Alternative |
|---|---|---|---|---|
| Strain | Identifier | Date of isolation | Origin | identifiers |
| Paenibacillus | LU17007 | 1 May 1996 | Germany, Tübingen, Wild- | DSM26970 |
| polymyxa | | | type isolate from soil | |
| Paenibacillus | LU21552 | Mutant generated in | Mutant successor of | — |
| polymyxa | | 2019 | LU17007 (NTG/UV | |
| | | | mutagenesis) | |
| Paenibacillus | DSM365 | before 22 August 1990, | Germany | — |
| polymyxa | | Isolate from garden- | Göttingen | |
| | | soil | | |

TABLE 5
other strains:

| | Strain | Date of | | Alternative |
|---|---|---|---|---|
| Strain | Identifier | isolation | Origin | identifiers |
| Rhizobium phaseoli | DSM30137 | before | country of origin | ATCC 14482, |
| | | 22 August 1990 | unknown | JCM 20683, |
| | | | | NRRL L-321, |
| | | | | VKM B-1966, |
| | | | | NBRC 14785 |
| Pseudomonas koreensis | IA- | 2016 | USA, isolated from | |
| | 250_TP_15 | | corn stalks | |
| Paraburkholderia phytofirmans | DSM17436 | before | Canada; isolated | LMG 22146, |
| | | 5 July 2005 | from surface- | CCUG 49060 |
| | | | sterilized onion roots | |
| Rhizobium radiobacter | DSM30205 | before | country of origin | ATCC 23308, |
| | | 22 August 1990 | unknown; isolated | CCM 1040, |
| | | | from | CIP B6, DSM |
| | | | Malus sp., crown | 60574, ICPBTT 3, |
| | | | gall of apple | NCPPB 397, |
| | | | seedling | NBRC |
| | | | | 102589 |
| Bradyrhizobium japonicum | 532C | 1960 | USA | SEMIA 5039, |
| | | | | 61A152 |
| Bacillus licheniformis | DSM13 | before | country of origin | ATCC 14580, |
| | | 21 August 1990 | unknown | CCM 2145, |
| | | | | IFO 12200, |
| | | | | NCIB 9375, |
| | | | | NCTC 10341, |
| | | | | NBRC 12200, |
| | | | | WDCM 00068 |
| Streptomyces lydicus | WYEC 108 | unkown | From market product | — |
| | | | Actinovate ® SP | |
| Bacillus subtilis | BU1814 | 2004 by | country of origin: | — |
| | | University | USA, Colorado | |
| | | of Delaware | | |
| Bacillus velezensis | MBI600 | 1988 by | country of origin: | — |
| | | Nottingham | GB, Nottinghamshire | |
| | | University | | |

### Example 1 Mutant Generation

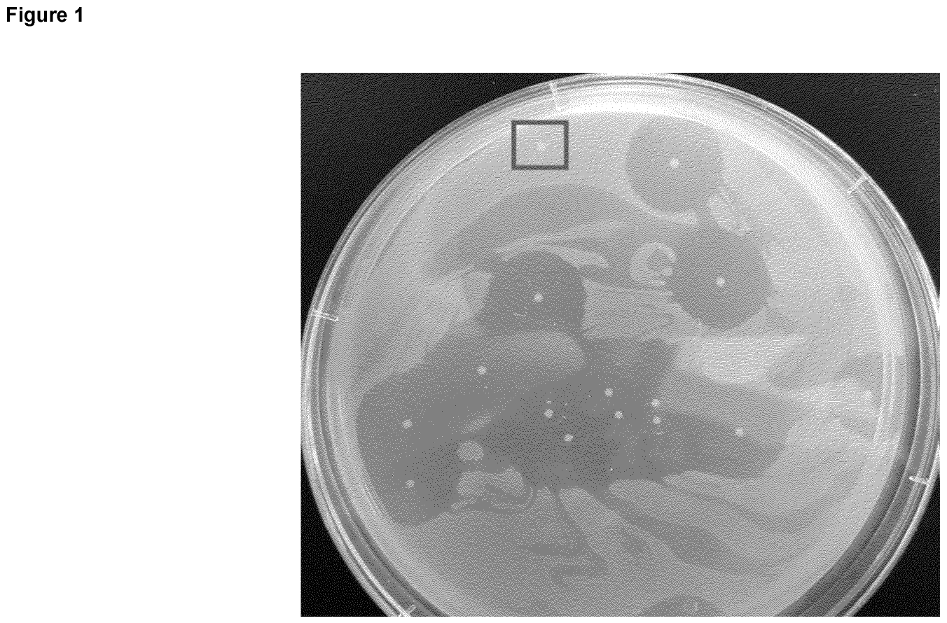

In order to abolish the production of antibiotics, such as polymyxin, from P. polymyxa LU17007, cells (7 E+10 CFU/mL) of this strain were exposed to N-methyl-N′-nitro-N-nitrosoguanidine (NTG) at a concentration of 400 μg/mL for 15 min. Mutants were initially screened for absence of inhibiting substances by overlaying single colonies with a top agar culture of Escherichia co/i which is known to be polymyxin-sensitive, followed by monitoring of inhibition zones, see FIG. 1. Mutant colonies of P. polymyxa, that did not inhibit growth of E. co/i on the agar plate were then cultivated in a soy-based liquid culture medium. Absence of polymyxin production in the culture broth was confirmed by LC-MS.

Besides using mutagenic agents, it was surprisingly found that environmental stress such as heat exposure or the presence of a fungus can also trigger a loss of polymyxin and tridecaptin production in Paenibacillus strains.

For the heat shock, 2 ml of a Paenibacillus LU20754 culture broth sample consisting of spores, endospores and viable cells derived from a 48h fermentation in soy-based medium was heated at 60° C. for 30 min. After this pretreatment, the culture sample was used for inoculation of a shake flask with soy-based medium. Cultivation took place for 72h (33° C., 250 ml shake flask with baffles, 40 ml filling volume, 150 rpm at 2.5 cm throw, pH 6.5±0.2). To investigate potential genetic changes compared to a parallel culture without heat shock, DNA extraction and sequencing was carried out. For this, the shake flask culture broths were used for inoculation of 12 ml Greiner tubes filled with 9 ml LB-medium to maintain a fresh culture before DNA extraction. The latter was done after 12h of cultivation using the Genomic tip Kit (Qiagen, Hilden, Germany) following manufacturer's instructions. DNA quality was assessed using agarose gel electrophoresis and DNA concentration was measured using Qubit 4 fluorometer (Thermo Fisher Scientific, Waltham, MA, USA), before DNA was sent for sequencing.

As an alternative external stressor, Paenibacillus LU20754 was also put into confrontation with fungal material. For this, the phytopathogenic strain Alternaria alternata was grown in liquid culture for 140h using a 1 L shake flask with baffles, 150 ml MEP medium (30 g/L malt-extract, 5 g/L mycological peptone, pH 5.4±0.2) at 23° C., 250 rpm/2.5 cm throw. Then, the fungal culture was pelleted by centrifugation at 13.000 g for 20 min. After resuspension in 40 ml water, sample was put in a homogenization device. To kill surviving fungal material, the sample was then placed in a ultrasonication bath for 60 min followed by an exposure to UV-light for >60 min. Afterwards, the fungal material was centrifuged again at 13.000 g for 20 min and the supernatant was discarded. The remaining pellet was lyophilized by freeze drying to obtain powdery material and stored at 4° C.

From this devitalized Alternaria cell powder, 2 g/L were added in a liquid culture of Paenibacillus LU20754. Cultivation of Paenibacillus took place for 72h in soy-based medium. DNA extraction for sequencing was carried out as described above.

### Example 2: Sequencing of Polymyxin Negative Candidates of LU17007 from Mutagenesis or Heat and Fungal Exposure

In order to identify genetic changes that may cause the inability of polymyxin synthesis of the mutant successors of wt strain P. polymyxa LU17007, whole genome sequencing was carried out for 20 individual strains. These strains showed no inhibiting zone in agar-based confrontation assays against the Gram-negative strain E. coli and revealed no polymyxin signals in LCMS analyses. In addition, candidate strains from the heat exposure or fungal confrontation were sent to sequencing. PacBio or Illumina sequencing technology was applied.

### PacBio Sequencing Parameters

For in-depth genetic analyses, Pacific Bioscience (PacBio) sequencing was performed exemplary with Paenibacillus culture broth samples obtained from the different stressing conditions as described before. Hence, an average of 360 thousand reads were sequenced per sample. Raw fastq sequencing data files were assembled using canu version 1.8 with parameters genomeSize=5.5m, corOutCoverage=60. This mostly resulted in two-contig genomes (a full chromosome and a short, ˜1 kb unplaced contig) with around 338× genome coverage after re-mapping. Genome annotations were carried over from the manually curated LU17007 genome annotations with a custom script. Biosynthetic gene clusters were annotated with antiSMASH 5.1.2 (Blin et al, 2019, antiSMASH 5.0: updates to the secondary metabolite genome mining pipeline, Nucleic Acids Research, Volume 47, Issue W1, Pages W81-W87, https://doi.org/10.1093/nar/gkz310).

### Illumina Sequencing Parameters

Sequencing of further 20 polymyxin-negative mutant successors of strain LU17007 was performed using Illumina Hi-Seq or Illumina MiniSeq. Between 1.5 and 7 Mio paired-end reads were sequenced per sample. The reads were preprocessed with Trimmomatic version 0.36 (Bolger et al, 2014, Trimmomatic: a flexible trimmer for Illumina sequence data. Bioinformatics 30(15), 2114-2120. https://doi.org/10.1093/bioinformatics/btu170): Clipping adapter sequences (settings: seed mismatches=2, palindrome clip threshold=30, simple clip threshold=10, min adapter length=5), Clipping leading and trailing bases with a quality below 21, Clipping before any stretch of 3 bases with an average quality below 20, Removing any read that, after the clipping steps, is shorter than 30 bp. Remaining read pairs with suitable overlap (minimum overlap of 25, maximum mismatch density of 0.1, maximum overlap of 150) were merged with FLASH v1.2.11 (Mago6 T, Salzberg SL, 2011, FLASH: fast length adjustment of short reads to improve genome assemblies. Bioinformatics. 1; 27(21):2957-63. doi: 10.1093/bioinformatics/btr507. Epub 2011 Sep. 7. PMID: 21903629; PMCID: PMC3198573). The genomes were assembled with SPAdes v3.13.0 (Nurk, et al, 2013, Assembling single-cell genomes and mini-metagenomes from chimeric MDA products. Journal of computational biology: a journal of computational molecular cell biology, 20(10), 714-737. https://doi.org/10.1089/cmb.2013.0084) using the high quality LU20754 PacBio assembly as reference. The assembled scaffolds were filtered with a custom script to remove scaffolds and contigs shorter than 500 bp, as well as PhiX sequencing control contig. Genome annotations were carried over from the manually curated LU17007 genome annotations with a custom script. Biosynthetic gene clusters were annotated with antiSMASH 5.1.2.

### Example 3: Mutation Analysis

Illumina reads were mapped to reference with BWA-MEM v0.7.17 (Heng Li, Richard Durbin, Fast and accurate long-read alignment with Burrows-Wheeler transform, Bioinformatics, Volume 26, Issue 5, 1 Mar. 2010, Pages 589-595). Indels and single nucleotide polymorphisms (SNPs) were called using the Genome Analysis Toolkit Lite (GATK) v2.3.9, with UnifiedGenotyper (McKenna A, Hanna M, Banks E, Sivachenko A, Cibulskis K, Kernytsky A, Garimella K, Altshuler D, Gabriel S, Daly M, DePristo M A. The Genome Analysis Toolkit: a MapReduce framework for analyzing next-generation DNA sequencing data. Genome Res. 2010 September; 20(9):1297-303. doi: 10.1101/gr.107524.110. Epub 2010 Jul. 19) with options ‘-glm both -mbq 25 -minIndelFrac 0.8’. The reference genome for genotyping was the ancestral strain LU17007.

### Example 4: Comparative Genomics

To investigate the cause of the inability of polymyxin synthesis in all pmx− strains, in-depth mutation analyses and comparative genomics were conducted. Surprisingly, mutation analyses based on the whole-genome data of each mutant strain did not show any non-silent mutation in the polymyxin gene cluster, its affiliated regulators or any other known genes involved in the polymyxin synthesis such as pmxA, pmxB, pmxC, pmxD, pmxE, spo0A, abrB, sfp. Also, genes for the supply and synthesis of polymyxin precursors and functional groups such as the amino acids L-Threonine, L-Leucine, D-Leucine, L-Aspartate semialdehyde or fatty acids (Yu Z, Qin W, Lin J, Fang S, Qiu J. Antibacterial mechanisms of polymyxin and bacterial resistance. Biomed Res Int. 2015; 2015:679109. doi: 10.1155/2015/679109. Epub 2015 Jan. 15) were not affected by non-silent or disruptive mutations.

However, comparative genomics of pmx− vs. pmx+ strains revealed that all of the 20 polymyxin negative strains had a roughly 289 kb smaller genome compared to the polymyxin producing strain LU17007. This was identified by mapping the reads of all mutant strain Illumina read bins onto the existing high-quality Pacbio assembly.

Analysis of the polymyxin negative strains showed that all of them were lacking a total of 147 coding sequences that are consecutively present on a single genome fragment in the wild-type strain. Here, many of these genes were only present as single copy in the early generation P. polymyxa LU17007.

One of these single copy genes included the diaminobutyrate-2-oxoglutarate transaminase gene ectB, necessary for L-2,4 diaminobutanoate (L-Dab) synthesis, which makes up six of the ten amino acid residues in polymyxin (Yu Z, Qin W, Lin J, Fang S, Qiu J. Antibacterial mechanisms of polymyxin and bacterial resistance. Biomed Res Int. 2015; 2015:679109. doi: 10.1155/2015/679109. Epub 2015 Jan. 15). It was hypothesized the inability to produce polymyxin by the mutant strains containing the smaller genome is due to the missing ectB gene and thus inability to produce Dab necessary for a functional polymyxin. This also explains why although the polymyxin gene cluster is intact and even transcribed in those strains, no polymyxin could be measured. Dab is also present in the antibiotic tridecaptin, suggesting that all mutant strains lacking the ectB gene did not only lose the ability to synthesize polymyxin, but also tridecaptin.

Based on the genome of the wt strain LU17007, AntiSMASH analyses revealed two NRPS clusters that show high similarity to the tridecaptin (lipopeptide antibiotic against gram-negative bacteria) synthase from Paenibacillus strain M-1. Remarkably, these tridecaptin clusters were found to be highly homologous and only present in the wt strain LU17007. Both clusters are surrounding the fragment carrying 147 genes that was lost in the polymyxin negative mutants of LU17007. Due to their high similarity (˜92% nt similarity) a recombination of both flanking clusters followed by a loss of the 289 kb genomic fragment in between was identified as a highly probable explanation since the mutant strains only showed a single tridecaptin cluster with a mixed sequence consisting of both tridecaptin clusters from the wildtype.

### Example 5: Inhibition Assay

In order to investigate whether the Paenibacillus mutants or the wild-type parent strain showed any inhibiting effect on growth of different Gram-positive and Gram-negative bacteria, an agar-based co-cultivation assay was performed.

Bacterial strains from Table 5 were transferred into 250 ml shake flasks containing 30 ml NB medium. Liquid cultivation was performed for 23 h at 30° C. and 250 rpm/2.5 cm throw. From this, 200 μL culture broth was spread on square NB agar plates and evenly distributed on the whole plate.

Paenibacillus strains from Table 7 were cultivated in 250 ml shake flask with 30 ml NB medium at 33° C. and 250 rpm/2.5 cm throw. After 32h cultivation, 2 ml of each liquid culture was transferred in a snap cap tube and was centrifugated for 1 min at 16.000 g. Then, supernatant was transferred through a 0.2 μm filter membrane in a fresh snap cap vial (=supernatant sample). In parallel, the remaining cell pellet was resuspended in 2 ml of a sterile 0.9% NaCl solution. After redissolving the cell pellet by gently mixing, the cell sample was further diluted 1:10 using 0.9% NaCl (=cell sample).

From this, 10 μL of each Paenibacillus supernatant or viable cell sample, respectively, were dropped on NB agar plates containing a culture from Table 6.

Cultivation of these agar plates was carried out for at least 72 days at 30° C. Microbial growth and formation of inhibition zones around the Paenibacillus culture or supernatant spots was assessed visually every day. The results were listed in Table 8.

TABLE 6
Bacterial strains used for detection of inhibiting
effects of Paenibacillus strains

| Strain | Strain Identifier | Gram staining |
|---|---|---|
| Rhizobium phaseoli | DSM30137 | negative |
| Pseudomonas koreensis | (IA-250_TP_15) | negative |
| Paraburkholderia phytofirmans | DSM17436 | negative |
| Rhizobium radiobacter | DSM30205 | negative |
| Bradyrhizobium japonicum | 532C | negative |
| Bacillus licheniformis | DSM13 | positive |
| Streptomyces lydicus | WYEC 108 | positive |
| Bacillus subtilis | BU1814 | positive |
| Bacillus velezensis | MBI600 | positive |

TABLE 7
Paenibacillus strains to test antimicrobial production

| | Strain | |
|---|---|---|
| Strain | Identifier | Origin |
| Paenibacillus polymyxa | LU17007 | Wild-type isolate from soil |
| Paenibacillus polymyxa | LU21552 | Mutant successor of |
| | | LU17007 (NTG/UV mutagenesis) |
| | | with recombination event |
| Paenibacillus polymyxa | DSM365 | DSMZ Leipzig, Germany. |
| | | Isolate from garden-soil |

TABLE 8
Results from the agar-based co-cultivation assay using Paenibacillus
strains combined with bacteria from other genera. Inhibition
of growth by the respective Paenibacillus strain is indicated
by “+”, whereas “−” means no repression of
growth of the listed bacterial strains by Paenibacillus.

| | Growth inhibition zone by Paenibacillus | | |
|---|---|---|---|
| Bacterial strain | LU17007 | DSM365 | LU21552 |
| Strain | (wt) | (wt) | (mutant) |
| Rhizobium phaseoli | + | + | − |
| DSM30137 | | | |
| Pseudomonas koreensis | + | + | − |
| (IA-250_TP_15) | | | |
| Paraburkholderia | + | + | − |
| phytofirmans | | | |
| DSM17436 | | | |
| Rhizobium radiobacter | + | + | − |
| DSM30205 | | | |
| Bradyrhizobium japonicum | + | + | − |
| Bacillus licheniformis | + | + | − |
| DSM13 | | | |
| Streptomyces lydicus | + | + | − |
| WYEC 108 | | | |
| B. subtilis | + | + | − |
| BU1814 | | | |
| B. velezensis | + | + | − |
| MBI600 | | | |

### Example 6: Analyses of Fusaricidins

To compare share of different Fusaricidins produced during cultivation, Paenibacillus strains from Table 9 were used for analyses via LC-MS. For sample generation, strains were cultivated for 72 h at 30° C. in glucose-starch-CaCO3 (GSC) medium as previously applied from Eliasson Lantz A, Jørgensen P, Poulsen E, Lindemann C, Oisson L. Determination of cell mass and polymyxin using multi-wavelength fluorescence. J Biotechnol. 2006 Feb. 24; 121(4):544-54. doi: 10.10161j.jbiotec.2005.08.007. Epub 2005 Sep. 12. and Niu B, Vater J, Rueckert C, Blom J, Lehmann M, Ru J J, Chen X H, Wang Q, Borriss R. Polymyxin P is the active principle in suppressing phytopathogenic Erwinia spp. by the biocontrol rhizobacterium Paenibacillus polymyxa M-1. BMC Microbiol. 2013 Jun. 18,13:137. doi: 10.1186/1471-2180-13-137. for antimicrobial substance analyses in Paenibacillus polymyxa strain POL4-2 (Alpharma ApS) and strain M-1, respectively. Cultivation took place at 150 rpm using 250 ml shake flask w/o baffles, containing 40 ml GSC medium as listed in Table 10.

TABLE 9
Strains used for LC-MS analyses of Fusaricidins

| | | Recombination of tridecaptin |
|---|---|---|
| Strain | Strain Identifier | clusters/lack of ectB |
| Paenibacillus polymyxa | LU17007 | No |
| Paenibacillus polymyxa | LU22381 | Yes |
| Paenibacillus polymyxa | LU21552 | Yes |

TABLE 10
composition of glucose-starch-CaCO3 (GSC) medium used for analyses
of fusaricidins in Paenibacillus strains from Table 9

| Compound | Amount (g/L) |
|---|---|
| Glucose | 20 |
| Starch | 20 |
| (NH₄)₂SO₄ | 20 |
| Yeast extract | 10 |
| K₂HPO₄ | 2.6 |
| FeSO₄•7H₂O | 0.1 |
| MgSO₄•7H₂O | 0.5 |
| NaCl | 0.25 |
| CaCO₃ | 9 |

### Sample Preparation for LC-hrMS:

After 72h cultivation time, culture broths of the strains from Table 9 were first centrifuged for 10 min at 20000×g. Obtained supernatants were separated and diluted 1:1 with acetonitrile/water (50:50, v/v) containing 0.1% formic acid (v/v). Resulting precipitation was separated by repeated centrifugation, and supernatants used for LC-hr MS analysis.

### LC-hrMS Methods

All measurements of these crude extracts were performed on a Thermo Vanquish Flex comprising a high-pressure gradient pump with a 150 μl mixing chamber. The method was based on separations with a BEH C18, 150×2.1 mm, 1.7 μm dp column (Waters, Eschborn, Germany). Two μl sample was separated by a multi-step linear gradient from (A) H2O+0.1% FA to (B) ACN+0.1% FA at a flow rate of 500 μL/min and 65° C. The gradient was initiated by a 5.0 min isocratic step at 15% B, followed by an increase to 50% B in 18 min, followed by a second increase to 98% B in 3 min to end up with a 5 min step at 98% B before re-equilibrations under the initial conditions. UV spectra were recorded by a DAD in the range from 200 to 600 nm with 2 nm width. The LC flow was splitted to 75 μL/min before entering the maXis II hr-ToF mass spectrometer (Bruker Daltonics, Germany) using the Apollo ESI source.

The ion source parameters were: capillary, 4000 V; endplate offset, 500 V; nebulizer, 1 bar; dry gas, 5 L/min; dry gas temperature, 200° C. Ion transfer parameters were: funnelRF, 400 Vpp; multipoleRF, 350 Vpp; quadrupole ion energy, 3.0 eV @ low m/z 150. Collision cell is set to 8 eV with a stepping collisionRF from 200 to 1200 Vpp and stepping transfer time from 50 to 100 μs with a timing ratio of 30 to 70%; pre puls storage, 7.0 μs in full scan mode. Mass spectra were acquired in positive auto MS/MS mode with selected precursor list ranging from 50-1500 m/z at a 10 Hz scan rate. Selected precursor list was created based on the theoretical masses of the expected m/z values of the fusaricidin derivatives. Each measurement started with the injection of a 20 μL plug of basic sodium formate solution, which is introduced by a loop that is

1. A Paenibacillus species strain comprising only one genetic locus comprising at least 80% sequence identity to a polynucleotide sequence of any one of SEQ IN NOs: 222, 223, 224, 225, 413, 414, 415, 416, 417, 418 and not comprising an open reading frame for a diaminobutyrate-2-oxoglutarate-transaminase comprising an amino acid sequence of at least 80% sequence identity to SEQ ID NO: 217.

2. A Paenibacillus species strain as claimed in claim 1, not comprising a polynucleotide sequence encoding an amino acid sequence comprising at least 80% sequence identity to SEQ ID NO: 1.

3. A Paenibacillus species strain as claimed in claim 1, being an Paenibacillus polymyxa strain and having an ANI-Value of at least 99.9% to the Paenibacillus polymyxa strain LU17007 deposited as DSM 26970 or to the Paenibacillus polymyxa strain SB3-1.

4. A Paenibacillus species strain as claimed in claim 1 being derived from a parent strain, wherein the parent strain comprises an open reading frame for a diaminobutyrate-2-oxoglutarate_transaminase comprising an amino acid sequence of at least 80% sequence identity to SEQ ID NO: 217 and at least two loci each one comprising at least 80% sequence identity to at least one of the nucleotide sequences of SEQ ID NO: 222, 223, 224, 225, 413, 414, 415, 416, 417 and 418.

5. A Paenibacillus species strain as claimed in claim 1 being derived from a parent strain, wherein the parent strain comprises an open reading frame encoding an amino acid sequence of at least 80% sequence identity to SEQ ID NO: 217 and an open reading frame encoding an amino acid sequence of at least 80% sequence identity to SEQ ID NO: 409, and an open reading frame encoding an amino acid sequence of at least 80% sequence identity to SEQ ID NO: 410.

6. A Paenibacillus species strain not comprising a polynucleotide sequence encoding an amino acid sequence of at least 80% sequence identity to SEQ ID NO: 1 and not comprising an open reading frame for a diaminobutyrate-2-oxoglutarate-transaminase comprising an amino acid sequence of at least 80% identity to SEQ ID NO: 217, derived from a parent strain, wherein the parent strain comprises an open reading frame for a diaminobutyrate-2-oxoglutarate-transaminase comprising an amino acid sequence of at least 80% sequence identity to SEQ ID NO: 217 and at least two loci each one comprising at least 80% sequence identity to at least one of the nucleotide sequences of SEQ ID NO: 222, 223, 224, 225, 413, 414, 415, 416, 417 and 418.

7. A Paenibacillus species strain as claimed in claim 1, being any one of the Paenibacillus polymyxa strains of LU20754 deposited as DSM 33970, LU21132 deposited as DSM 33971, LU21514 deposited as DSM 33972, LU21552 deposited as DSM 33973, LU22377 deposited as DSM 33974, LU22378 deposited as DSM 33975, LU22379 deposited as DSM 33976, and LU22381 deposited as DSM 33977.

8. A mixture comprising a) a pure culture of a Paenibacillus species strain which does not comprise an open reading frame for a diaminobutyrate-2-oxoglutarate-transaminase comprising an amino acid sequence of at least 90% sequence identity to at least one of SEQ ID NO: 217, 218, 219, 220 or 221 and b) a pure culture of a microorganism which is sensitive to at least one of polymyxin, tridecaptin, polypeptin, paenilipoheptin, octapeptin, jolipeptin, gavaserin or saltavalin.

9. A mixture as claimed in claim 8, wherein the Paenibacillus species strain of a) comprises no or only one locus encoding an amino acid sequence comprising at least 90% sequence identity to SEQ ID NO: 1 and does not produce a polymyxin or a tridecaptin.

10. A mixture as claimed in claim 8, wherein the parent strain of the Paenibacillus species strain of a) comprises at least one locus encoding an amino acid sequence comprising at least 90% sequence identity to SEQ ID NO: 1 and does produce at least one tridecaptin.

11. A mixture as claimed in claim 8 comprising a pure culture of a of a microorganism of b) selected from Azotobacter, Azospirillum, Pseudomonas, Paraburkholderia, Rhizobium, Bradyrhizobium, Bacillus or Streptomyces.

12. A mixture as claimed in claim 8 comprising a pure culture of a microorganism of b) selected from: Pseudomonas koreensis, Pseudomonas gessardii, Pseudomonas chlororaphis, Paraburkholderia phytofirmans, Rhizobium phaseoli, Rhizobium radiobacter, Bradyrhizobium japonicum, Bacillus licheniformis, Bacillus subtilis, Bacillus amyloliquefaciens, Bacillus licheniformis, Bacillus mycoides, Bacillus firmus, Bacillus thuringiensis, Bacillus pumilus, Bacillus simplex, Streptomyces griseoviridis and Streptomyces lydicus.

13. An agrochemical composition comprising a Paenibacillus species strain as claimed in claim 1.

14. A method for controlling, suppressing or preventing fungal infection of plants comprising the steps of a) providing a pure culture of a Paenibacillus species strain as claimed in claim 1 and b) applying an effective amount of the a pure culture of a Paenibacillus species strain to the plant, pathogenic fungi, their habitat, plant propagation material or the soil used to grow the plant.

15. A method to enhance plant growth comprising the steps of a) providing a pure culture of a Paenibacillus species strain as claimed in claim 1 and b) applying an effective amount of the pure culture of a Paenibacillus species strain to the plant, pathogenic fungi, their habitat, plant propagation material or the soil used to grow the plant.
